(19) United States
(12) Patent Application Publication
(10) Pub. No.: US 2023/0041989 A1
(43) Pub. Date: Feb. 9, 2023

(54) BASE CALLING USING MULTIPLE BASE CALLER MODELS

(71) Applicant: ILLUMINA SOFTWARE, INC., San Diego, CA (US)

(72) Inventors: Gavin Derek PARNABY, Laguna Niguel, CA (US); Mark David HAHM, Hartland, WI (US)

(73) Assignee: ILLUMINA SOFTWARE, INC., San Diego, CA (US)

(21) Appl. No.: 17/876,528

(22) Filed: Jul. 28, 2022

Related U.S. Application Data
(60) Provisional application No. 63/228,954, filed on Aug. 3, 2021.

(51) Int. Cl. G16B 30/00; G16B 40/00; G06T 7/00

(52) U.S. Cl. CPC G16B 30/00; G16B 40/00; G06T 7/0002

(57) ABSTRACT

A method of base calling using at least two base callers is disclosed. The method includes executing at least a first base caller and a second base caller on sensor data generated for sensing cycles in a series of sensing cycles; generating, by the first base caller, first classification information associated with the sensor data, based on executing the first base caller on the sensor data; and generating, by the second base caller, second classification information associated with the sensor data, based on executing the second base caller on the sensor data. In an example, based on the first classification information and the second classification information, a final classification information is generated, where the final classification information includes one or more base calls for the sensor data.

### PRIORITY APPLICATION

This application claims the benefit of U.S. Provisional Patent Application No. 63/228,954, titled “BASE CALLING USING MULTIPLE BASE CALLER MODELS,” filed Aug. 3, 2021 (Attorney Docket No. ILLM 1021-1/IP-1856-PRV). The priority application is hereby incorporated by reference for all purposes.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep neural networks such as deep convolutional neural networks for analyzing data.

### INCORPORATIONS

The following are incorporated by reference as if fully set forth herein:

U.S. Provisional Patent Application No. 62/979,384, titled “ARTIFICIAL INTELLIGENCE-BASED BASE CALLING OF INDEX SEQUENCES,” filed 20 Feb. 2020 (Attorney Docket No. ILLM 1015-1/IP-1857-PRV);

U.S. Provisional Patent Application No. 62/979,414, titled “ARTIFICIAL INTELLIGENCE-BASED MANY-TO-MANY BASE CALLING,” filed 20 Feb. 2020 (Attorney Docket No. ILLM 1016-1/IP-1858-PRV);

U.S. Nonprovisional patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US);

U.S. Nonprovisional patent application Ser. No. 16/825,991, titled “ARTIFICIAL INTELLIGENCE-BASED GENERATION OF SEQUENCING METADATA,” filed 20 Mar. 2020 (Attorney Docket No. ILLM 1008-17/IP-1741-US);

U.S. Nonprovisional patent application Ser. No. 16/826,126, titled “ARTIFICIAL INTELLIGENCE-BASED BASE CALLING,” filed 20 Mar. 2020 (Attorney Docket No. ILLM 1008-18/IP-1744-US);

U.S. Nonprovisional patent application Ser. No. 16/826,134, titled “ARTIFICIAL INTELLIGENCE-BASED QUALITY SCORING,” filed 20 Mar. 2020 (Attorney Docket No. ILLM 1008-19/IP-1747-US); and

U.S. Nonprovisional patent application Ser. No. 16/826,168, titled “ARTIFICIAL INTELLIGENCE-BASED SEQUENCING,” filed 21 Mar. 2020 (Attorney Docket No. ILLM 1008-20/IP-1752-PRV-US);

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

The rapid improvement in computation capability has made deep Convolution Neural Networks (CNNs) a great success in recent years on many computer vision tasks with significantly improved accuracy. During the inference phase, many applications demand low latency processing of one image with strict power consumption requirement, which reduces the efficiency of Graphics Processing Unit (GPU) and other general-purpose platform, bringing opportunities for specific acceleration hardware, e.g., Field Programmable Gate Array (FPGA), by customizing the digital circuit specific for the deep learning algorithm inference. However, deploying CNNs on portable and embedded systems is still challenging due to large data volume, intensive computation, varying algorithm structures, and frequent memory accesses.

As convolution contributes most operations in CNNs, the convolution acceleration scheme significantly affects the efficiency and performance of a hardware CNN accelerator. Convolution involves multiply and accumulate (MAC) operations with four levels of loops that slide along kernel and feature maps. The first loop level computes the MAC of pixels within a kernel window. The second loop level accumulates the sum of products of the MAC across different input feature maps. After finishing the first and second loop levels, a final output element in the output feature map is obtained by adding the bias. The third loop level slides the kernel window within an input feature map. The fourth loop level generates different output feature maps.

FPGAs have gained increasing interest and popularity in particular to accelerate inference tasks, due to their (1) high degree of reconfigurability, (2) faster development time compared to Application Specific Integrated Circuits (ASICs) to catch up with the rapid evolving of CNNs, (3) good performance, and (4) superior energy efficiency compared to GPUs. The high performance and efficiency of an FPGA can be realized by synthesizing a circuit that is customized for a specific computation to directly process billions of operations with the customized memory systems. For instance, hundreds to thousands of digital signal processing (DSP) blocks on modern FPGAs support the core convolution operation, e.g., multiplication and addition, with high parallelism. Dedicated data buffers between external on-chip memory and on-chip processing engines (PEs) can be designed to realize the preferred dataflow by configuring tens of Mbyte on-chip block random access memories (BRAM) on the FPGA chip.

Efficient dataflow and hardware architecture of CNN acceleration are desired to minimize data communication while maximizing resource utilization to achieve high performance. An opportunity arises to design methodology and framework to accelerate the inference process of various CNN algorithms on acceleration hardware with high performance, efficiency, and flexibility.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 illustrates a cross-section of a biosensor that can be used in various embodiments.

FIG. 2 depicts one implementation of a flow cell that contains clusters in its tiles.

FIG. 3 illustrates an example flow cell with eight lanes, and also illustrates a zoom-in on one tile and its clusters and their surrounding background.

FIG. 4 is a simplified block diagram of the system for analysis of sensor data from a sequencing system, such as base call sensor outputs.

FIG. 5 is a simplified diagram showing aspects of the base calling operation, including functions of a runtime program executed by a host processor.

FIG. 6 is a simplified diagram of a configuration of a configurable processor such as that of FIG. 4.

FIG. 6A illustrates a system that employs two or more base callers for base calling operations on the raw images output by a biosensor.

FIG. 7 is a diagram of a neural network architecture which can be executed using a configurable or a reconfigurable array configured as described herein.

FIG. 8A is a simplified illustration of an organization of tiles of sensor data used by a neural network architecture like that of FIG. 7.

FIG. 8B is a simplified illustration of patches of tiles of sensor data used by a neural network architecture like that of FIG. 7.

FIG. 9 illustrates part of a configuration for a neural network like that of FIG. 7 on a configurable or a reconfigurable array, such as a Field Programmable Gate Array (FPGA).

FIG. 10 is a diagram of another alternative neural network architecture which can be executed using a configurable or a reconfigurable array configured as described herein.

FIG. 11 illustrates one implementation of a specialized architecture of the neural network-based base caller that is used to segregate processing of data for different sequencing cycles.

FIG. 12 depicts one implementation of segregated layers, each of which can include convolutions.

FIG. 13A depicts one implementation of combinatory layers, each of which can include convolutions.

FIG. 13B depicts another implementation of the combinatory layers, each of which can include convolutions.

FIG. 14 illustrates a base calling system comprising a plurality of base callers, to predict base calls of an unknown analyte comprising base sequences.

FIGS. 15A, 15B, 15C, 15D, and 15E illustrate corresponding flow charts that depict various operations of the base calling system of FIG. 14 for corresponding sets of sensor data.

FIG. 16 illustrates a context information generation module of the base calling system of FIG. 14 generating context information for an example set of sensor data.

FIG. 17A illustrates a flow cell comprising tiles that are categorized based on spatial location of the tiles.

FIG. 17B illustrates a tile of a flow cell comprising clusters that are categorized based on spatial location of the clusters.

FIG. 17C illustrates an example of fading, in which signal intensity is decreased as a function of cycle number is a sequencing run of a base calling operation.

FIG. 17D conceptually illustrates a decreasing signal-to-noise ratio as cycles of sequencing progress.

FIG. 18 shows base calling accuracy (1-base calling error rate) of the different example configurations of base caller, over base calling homopolymers (e.g., GGGGG) and near-homopolymers (e.g., GGTGG).

FIG. 19A illustrates generation of final base call for a set of sensor data, based on a function of first base call classification information from a first base caller and second base call classification information from a second base caller of the base calling system of FIG. 14.

FIG. 19A1 illustrates a Look Up Table (LUT) indicating an example weightage scheme to be used for final confidence scores, based on temporal context information.

FIG. 19B illustrates a LUT indicating a base caller to be used, when the called bases include a special base sequence.

FIG. 19C illustrates a LUT indicating weightage given to confidence scores of individual base callers, when the called bases include a special base sequence.

FIG. 19D illustrates a LUT indicating operation of the base calling combining module of FIG. 14, in view of detection of one or more bubbles in a cluster of the flow cell.

FIG. 19D1 illustrates a LUT indicating operation of the base calling combining module of FIG. 14, in view of detection of out of focus image(s) from a cluster of the flow cell.

FIG. 19E illustrates a LUT indicating example weightages given to confidence scores of individual base callers, based on groups of reagents used.

FIG. 19F illustrates a LUT indicating operation of the base calling combining module of FIG. 14, in view of spatial classification of the tiles.

FIG. 19G illustrates a LUT indicating operation of the base calling combining module of FIG. 14, in view of spatial classification of the clusters.

FIG. 20A illustrates a LUT indicating operation of the base calling combining module of FIG. 14 when (i) a special base sequence is detected and (ii) a first called base from the first base caller does not match with a second called base from the second base caller.

FIG. 20B illustrates a LUT indicating operation of the base calling combining module of FIG. 14 when (i) a bubble is detected in a cluster and (ii) the first called base from the first base caller does not match with second called base from the second base caller.

FIG. 20C illustrates a LUT indicating operation of the base calling combining module of FIG. 14 when (i) one or more out of focus images are detected from at least one cluster and (ii) the first called base from the first base caller does not match with second called base from the second base caller.

FIG. 20D illustrates a LUT indicating operation of the base calling combining module of FIG. 14 when (i) the sensor data is from an edge cluster and (ii) the first called base from the first base caller does not match with second called base from the second base caller.

FIG. 21 illustrates a base calling system comprising a plurality of base callers, to predict base calls of unknown analyte comprising base sequences, where a neural network based final base call determination module determines the final base calls based on outputs of one or more of the plurality of base callers.

FIG. 22 is a block diagram of a base calling system in accordance with one implementation.

FIG. 23 is a block diagram of a system controller that can be used in the system of FIG. 22.

FIG. 24 is a simplified block diagram of a computer system that can be used to implement the technology disclosed.

### DETAILED DESCRIPTION

As used herein, the terms “polynucleotide” or “nucleic acids” refer to deoxyribonucleic acid (DNA), but where appropriate the skilled artisan will recognize that the systems and devices herein can also be utilized with ribonucleic acid (RNA). The terms should be understood to include, as equivalents, analogs of either DNA or RNA made from nucleotide analogs. The terms as used herein also encompasses cDNA, that is complementary, or copy, DNA produced from an RNA template, for example by the action of reverse transcriptase.

The single stranded polynucleotide molecules sequenced by the systems and devices herein can have originated in single-stranded form, as DNA or RNA or have originated in double-stranded DNA (dsDNA) form (e.g., genomic DNA fragments, PCR and amplification products and the like). Thus, a single stranded polynucleotide may be the sense or antisense strand of a polynucleotide duplex. Methods of preparation of single stranded polynucleotide molecules suitable for use in the method of the disclosure using standard techniques are well known in the art. The precise sequence of the primary polynucleotide molecules is generally not material to the disclosure, and may be known or unknown. The single stranded polynucleotide molecules can represent genomic DNA molecules (e.g., human genomic DNA) including both intron and exon sequences (coding sequence), as well as non-coding regulatory sequences such as promoter and enhancer sequences.

In certain embodiments, the nucleic acid to be sequenced through use of the current disclosure is immobilized upon a substrate (e.g., a substrate within a flowcell or one or more beads upon a substrate such as a flowcell, etc.). The term “immobilized” as used herein is intended to encompass direct or indirect, covalent or non-covalent attachment, unless indicated otherwise, either explicitly or by context. In certain embodiments covalent attachment may be preferred, but generally all that is required is that the molecules (e.g. nucleic acids) remain immobilized or attached to the support under conditions in which it is intended to use the support, for example in applications requiring nucleic acid sequencing.

The term “solid support” (or “substrate” in certain usages) as used herein refers to any inert substrate or matrix to which nucleic acids can be attached, such as for example glass surfaces, plastic surfaces, latex, dextran, polystyrene surfaces, polypropylene surfaces, polyacrylamide gels, gold surfaces, and silicon wafers. In many embodiments, the solid support is a glass surface (e.g., the planar surface of a flowcell channel). In certain embodiments the solid support may comprise an inert substrate or matrix which has been “functionalized,” for example by the application of a layer or coating of an intermediate material comprising reactive groups which permit covalent attachment to molecules such as polynucleotides. By way of non-limiting example such supports can include polyacrylamide hydrogels supported on an inert substrate such as glass. In such embodiments the molecules (polynucleotides) can be directly covalently attached to the intermediate material (e.g., the hydrogel) but the intermediate material can itself be non-covalently attached to the substrate or matrix (e.g., the glass substrate). Covalent attachment to a solid support is to be interpreted accordingly as encompassing this type of arrangement.

As indicated above, the present disclosure comprises novel systems and devices for sequencing nucleic acids. As will be apparent to those of skill in the art, references herein to a particular nucleic acid sequence may, depending on the context, also refer to nucleic acid molecules which comprise such nucleic acid sequence. Sequencing of a target fragment means that a read of the chronological order of bases is established. The bases that are read do not need to be contiguous, although this is preferred, nor does every base on the entire fragment have to be sequenced during the sequencing. Sequencing can be carried out using any suitable sequencing technique, wherein nucleotides or oligonucleotides are added successively to a free 3′ hydroxyl group, resulting in synthesis of a polynucleotide chain in the 5′ to 3′ direction. The nature of the nucleotide added is preferably determined after each nucleotide addition. Sequencing techniques using sequencing by ligation, wherein not every contiguous base is sequenced, and techniques such as massively parallel signature sequencing (MPSS) where bases are removed from, rather than added to, the strands on the surface are also amenable to use with the systems and devices of the disclosure.

In certain embodiments, the current disclosure discloses sequencing-by-synthesis (SBS). In SBS, four fluorescently labeled modified nucleotides are used to sequence dense clusters of amplified DNA (possibly millions of clusters) present on the surface of a substrate (e.g., a flowcell). Various additional aspects regarding SBS procedures and methods, which can be utilized with the systems and devices herein, are disclosed in, for example, WO04018497, WO04018493 and U.S. Pat. No. 7,057,026 (nucleotides), WO05024010 and WO06120433 (polymerases), WO05065814 (surface attachment techniques), and WO 9844151, WO06064199 and WO07010251, the contents of each of which are incorporated herein by reference in their entirety.

In particular uses of the systems/devices herein the flowcells containing the nucleic acid samples for sequencing are placed within the appropriate flowcell holder. The samples for sequencing can take the form of single molecules, amplified single molecules in the form of clusters, or beads comprising molecules of nucleic acid. The nucleic acids are prepared such that they comprise an oligonucleotide primer adjacent to an unknown target sequence. To initiate the first SBS sequencing cycle, one or more differently labeled nucleotides, and DNA polymerase, etc., are flowed into/through the flowcell by the fluid flow subsystem (various embodiments of which are described herein). Either a single nucleotide can be added at a time, or the nucleotides used in the sequencing procedure can be specially designed to possess a reversible termination property, thus allowing each cycle of the sequencing reaction to occur simultaneously in the presence of all four labeled nucleotides (A, C, T, G). Where the four nucleotides are mixed together, the polymerase is able to select the correct base to incorporate and each sequence is extended by a single base. In such methods of using the systems, the natural competition between all four alternatives leads to higher accuracy than wherein only one nucleotide is present in the reaction mixture (where most of the sequences are therefore not exposed to the correct nucleotide). Sequences where a particular base is repeated one after another (e.g., homopolymers) are addressed like any other sequence and with high accuracy.

The fluid flow subsystem also flows the appropriate reagents to remove the blocked 3′ terminus (if appropriate) and the fluorophore from each incorporated base. The substrate can be exposed either to a second round of the four blocked nucleotides, or optionally to a second round with a different individual nucleotide. Such cycles are then repeated, and the sequence of each cluster is read over the multiple chemistry cycles. The computer aspect of the current disclosure can optionally align the sequence data gathered from each single molecule, cluster or bead to determine the sequence of longer polymers, etc. Alternatively, the image processing and alignment can be performed on a separate computer.

The heating/cooling components of the system regulate the reaction conditions within the flowcell channels and reagent storage areas/containers (and optionally the camera, optics, and/or other components), while the fluid flow components allow the substrate surface to be exposed to suitable reagents for incorporation (e.g., the appropriate fluorescently labeled nucleotides to be incorporated) while unincorporated reagents are rinsed away. An optional movable stage upon which the flowcell is placed allows the flowcell to be brought into proper orientation for laser (or other light) excitation of the substrate and optionally moved in relation to a lens objective to allow reading of different areas of the substrate. Additionally, other components of the system are also optionally movable/adjustable (e.g., the camera, the lens objective, the heater/cooler, etc.). During laser excitation, the image/location of emitted fluorescence from the nucleic acids on the substrate is captured by the camera component, thereby, recording the identity, in the computer component, of the first base for each single molecule, cluster or bead.

Embodiments described herein may be used in various biological or chemical processes and systems for academic or commercial analysis. More specifically, embodiments described herein may be used in various processes and systems where it is desired to detect an event, property, quality, or characteristic that is indicative of a desired reaction. For example, embodiments described herein include cartridges, biosensors, and their components as well as bioassay systems that operate with cartridges and biosensors. In particular embodiments, the cartridges and biosensors include a flow cell and one or more sensors, pixels, light detectors, or photodiodes that are coupled together in a substantially unitary structure.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” or “including” an element or a plurality of elements having a particular property may include additional elements whether or not they have that property.

As used herein, a “desired reaction” includes a change in at least one of a chemical, electrical, physical, or optical property (or quality) of an analyte-of-interest. In particular embodiments, the desired reaction is a positive binding event (e.g., incorporation of a fluorescently labeled biomolecule with the analyte-of-interest). More generally, the desired reaction may be a chemical transformation, chemical change, or chemical interaction. The desired reaction may also be a change in electrical properties. For example, the desired reaction may be a change in ion concentration within a solution. Exemplary reactions include, but are not limited to, chemical reactions such as reduction, oxidation, addition, elimination, rearrangement, esterification, amidation, etherification, cyclization, or substitution; binding interactions in which a first chemical binds to a second chemical; dissociation reactions in which two or more chemicals detach from each other; fluorescence; luminescence; bioluminescence; chemiluminescence; and biological reactions, such as nucleic acid replication, nucleic acid amplification, nucleic acid hybridization, nucleic acid ligation, phosphorylation, enzymatic catalysis, receptor binding, or ligand binding. The desired reaction can also be an addition or elimination of a proton, for example, detectable as a change in pH of a surrounding solution or environment. An additional desired reaction can be detecting the flow of ions across a membrane (e.g., natural or synthetic bilayer membrane), for example as ions flow through a membrane the current is disrupted and the disruption can be detected.

In particular embodiments, the desired reaction includes the incorporation of a fluorescently-labeled molecule to an analyte. The analyte may be an oligonucleotide and the fluorescently-labeled molecule may be a nucleotide. The desired reaction may be detected when an excitation light is directed toward the oligonucleotide having the labeled nucleotide, and the fluorophore emits a detectable fluorescent signal. In alternative embodiments, the detected fluorescence is a result of chemiluminescence or bioluminescence. A desired reaction may also increase fluorescence (or Forster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

As used herein, a “reaction component” or “reactant” includes any substance that may be used to obtain a desired reaction. For example, reaction components include reagents, enzymes, samples, other biomolecules, and buffer solutions. The reaction components are typically delivered to a reaction site in a solution and/or immobilized at a reaction site. The reaction components may interact directly or indirectly with another substance, such as the analyte-of-interest.

As used herein, the term “reaction site” is a localized region where a desired reaction may occur. A reaction site may include support surfaces of a substrate where a substance may be immobilized thereon. For example, a reaction site may include a substantially planar surface in a channel of a flow cell that has a colony of nucleic acids thereon. Typically, but not always, the nucleic acids in the colony have the same sequence, being for example, clonal copies of a single stranded or double stranded template. However, in some embodiments a reaction site may contain only a single nucleic acid molecule, for example, in a single stranded or double stranded form. Furthermore, a plurality of reaction sites may be unevenly distributed along the support surface or arranged in a predetermined manner (e.g., side-by-side in a matrix, such as in microarrays). A reaction site can also include a reaction chamber (or well) that at least partially defines a spatial region or volume configured to compartmentalize the desired reaction.

This application uses the terms “reaction chamber” and “well” interchangeably. As used herein, the term “reaction chamber” or “well” includes a spatial region that is in fluid communication with a flow channel. The reaction chamber may be at least partially separated from the surrounding environment or other spatial regions. For example, a plurality of reaction chambers may be separated from each other by shared walls. As a more specific example, the reaction chamber may include a cavity defined by interior surfaces of a well and have an opening or aperture so that the cavity may be in fluid communication with a flow channel. Biosensors including such reaction chambers are described in greater detail in international application no. PCT/US2011/057111, filed on Oct. 20, 2011, which is incorporated herein by reference in its entirety.

In some embodiments, the reaction chambers are sized and shaped relative to solids (including semi-solids) so that the solids may be inserted, fully or partially, therein. For example, the reaction chamber may be sized and shaped to accommodate only one capture bead. The capture bead may have clonally amplified DNA or other substances thereon. Alternatively, the reaction chamber may be sized and shaped to receive an approximate number of beads or solid substrates. As another example, the reaction chambers may also be filled with a porous gel or substance that is configured to control diffusion or filter fluids that may flow into the reaction chamber.

In some embodiments, sensors (e.g., light detectors, photodiodes) are associated with corresponding pixel areas of a sample surface of a biosensor. As such, a pixel area is a geometrical construct that represents an area on the biosensor's sample surface for one sensor (or pixel). A sensor that is associated with a pixel area detects light emissions gathered from the associated pixel area when a desired reaction has occurred at a reaction site or a reaction chamber overlying the associated pixel area. In a flat surface embodiment, the pixel areas can overlap. In some cases, a plurality of sensors may be associated with a single reaction site or a single reaction chamber. In other cases, a single sensor may be associated with a group of reaction sites or a group of reaction chambers.

As used herein, a “biosensor” includes a structure having a plurality of reaction sites and/or reaction chambers (or wells). A biosensor may include a solid-state imaging device (e.g., CCD or CMOS imager) and, optionally, a flow cell mounted thereto. The flow cell may include at least one flow channel that is in fluid communication with the reaction sites and/or the reaction chambers. As one specific example, the biosensor is configured to fluidically and electrically couple to a bioassay system. The bioassay system may deliver reactants to the reaction sites and/or the reaction chambers according to a predetermined protocol (e.g., sequencing-by-synthesis) and perform a plurality of imaging events. For example, the bioassay system may direct solutions to flow along the reaction sites and/or the reaction chambers. At least one of the solutions may include four types of nucleotides having the same or different fluorescent labels. The nucleotides may bind to corresponding oligonucleotides located at the reaction sites and/or the reaction chambers. The bioassay system may then illuminate the reaction sites and/or the reaction chambers using an excitation light source (e.g., solid-state light sources, such as light-emitting diodes or LEDs). The excitation light may have a predetermined wavelength or wavelengths, including a range of wavelengths. The excited fluorescent labels provide emission signals that may be captured by the sensors.

In alternative embodiments, the biosensor may include electrodes or other types of sensors configured to detect other identifiable properties. For example, the sensors may be configured to detect a change in ion concentration. In another example, the sensors may be configured to detect the ion current flow across a membrane.

As used herein, a “cluster” is a colony of similar or identical molecules or nucleotide sequences or DNA strands. For example, a cluster can be an amplified oligonucleotide or any other group of a polynucleotide or polypeptide with a same or similar sequence. In other embodiments, a cluster can be any element or group of elements that occupy a physical area on a sample surface. In embodiments, clusters are immobilized to a reaction site and/or a reaction chamber during a base calling cycle.

As used herein, the term “immobilized,” when used with respect to a biomolecule or biological or chemical substance, includes substantially attaching the biomolecule or biological or chemical substance at a molecular level to a surface. For example, a biomolecule or biological or chemical substance may be immobilized to a surface of the substrate material using adsorption techniques including non-covalent interactions (e.g., electrostatic forces, van der Waals, and dehydration of hydrophobic interfaces) and covalent binding techniques where functional groups or linkers facilitate attaching the biomolecules to the surface Immobilizing biomolecules or biological or chemical substances to a surface of a substrate material may be based upon the properties of the substrate surface, the liquid medium carrying the biomolecule or biological or chemical substance, and the properties of the biomolecules or biological or chemical substances themselves. In some cases, a substrate surface may be functionalized (e.g., chemically or physically modified) to facilitate immobilizing the biomolecules (or biological or chemical substances) to the substrate surface. The substrate surface may be first modified to have functional groups bound to the surface. The functional groups may then bind to biomolecules or biological or chemical substances to immobilize them thereon. A substance can be immobilized to a surface via a gel, for example, as described in US Patent Publ. No. US 2011/0059865 A1, which is incorporated herein by reference.

In some embodiments, nucleic acids can be attached to a surface and amplified using bridge amplification. Useful bridge amplification methods are described, for example, in U.S. Pat. No. 5,641,658; WO 2007/010251; U.S. Pat. No. 6,090,592; U.S. Patent Publ. No. 2002/0055100 A1; U.S. Pat. No. 7,115,400; U.S. Patent Publ. No. 2004/0096853 A1; U.S. Patent Publ. No. 2004/0002090 A1; U.S. Patent Publ. No. 2007/0128624 A1; and U.S. Patent Publ. No. 2008/0009420 A1, each of which is incorporated herein in its entirety. Another useful method for amplifying nucleic acids on a surface is Rolling Circle Amplification (RCA), for example, using methods set forth in further detail below. In some embodiments, the nucleic acids can be attached to a surface and amplified using one or more primer pairs. For example, one of the primers can be in solution and the other primer can be immobilized on the surface (e.g., 5′-attached). By way of example, a nucleic acid molecule can hybridize to one of the primers on the surface followed by extension of the immobilized primer to produce a first copy of the nucleic acid. The primer in solution then hybridizes to the first copy of the nucleic acid which can be extended using the first copy of the nucleic acid as a template. Optionally, after the first copy of the nucleic acid is produced, the original nucleic acid molecule can hybridize to a second immobilized primer on the surface and can be extended at the same time or after the primer in solution is extended. In any embodiment, repeated rounds of extension (e.g., amplification) using the immobilized primer and primer in solution provide multiple copies of the nucleic acid.

In particular embodiments, the assay protocols executed by the systems and methods described herein include the use of natural nucleotides and also enzymes that are configured to interact with the natural nucleotides. Natural nucleotides include, for example, ribonucleotides (RNA) or deoxyribonucleotides (DNA). Natural nucleotides can be in the mono-, di-, or tri-phosphate form and can have a base selected from adenine (A), thymine (T), uracil (U), guanine (G) or cytosine (C). It will be understood however that non-natural nucleotides, modified nucleotides or analogs of the aforementioned nucleotides can be used. Some examples of useful non-natural nucleotides are set forth below in regard to reversible terminator-based sequencing by synthesis methods.

In embodiments that include reaction chambers, items or solid substances (including semi-solid substances) may be disposed within the reaction chambers. When disposed, the item or solid may be physically held or immobilized within the reaction chamber through an interference fit, adhesion, or entrapment. Exemplary items or solids that may be disposed within the reaction chambers include polymer beads, pellets, agarose gel, powders, quantum dots, or other solids that may be compressed and/or held within the reaction chamber. In particular embodiments, a nucleic acid superstructure, such as a DNA ball, can be disposed in or at a reaction chamber, for example, by attachment to an interior surface of the reaction chamber or by residence in a liquid within the reaction chamber. A DNA ball or other nucleic acid superstructure can be preformed and then disposed in or at the reaction chamber. Alternatively, a DNA ball can be synthesized in situ at the reaction chamber. A DNA ball can be synthesized by rolling circle amplification to produce a concatemer of a particular nucleic acid sequence and the concatemer can be treated with conditions that form a relatively compact ball. DNA balls and methods for their synthesis are described, for example in, U.S. Patent Publication Nos. 2008/0242560 A1 or 2008/0234136 A1, each of which is incorporated herein in its entirety. A substance that is held or disposed in a reaction chamber can be in a solid, liquid, or gaseous state.

As used herein, “base calling” identifies a nucleotide base in a nucleic acid sequence. Base calling refers to the process of determining a base call (A, C, G, T) for every cluster at a specific cycle. As an example, base calling can be performed utilizing four-channel, two-channel or one-channel methods and systems described in the incorporated materials of U.S. Patent Application Publication No. 2013/0079232. In particular embodiments, a base calling cycle is referred to as a “sampling event.” In one dye and two-channel sequencing protocol, a sampling event comprises two illumination stages in time sequence, such that a pixel signal is generated at each stage. The first illumination stage induces illumination from a given cluster indicating nucleotide bases A and T in a AT pixel signal, and the second illumination stage induces illumination from a given cluster indicating nucleotide bases C and T in a CT pixel signal.

The technology disclosed, e.g., the disclosed base callers can be implemented on processors like Central Processing Units (CPUs), Graphics Processing Units (GPUs), Field Programmable Gate Arrays (FPGAs), Coarse-Grained Reconfigurable Architectures (CGRAs), Application-Specific Integrated Circuits (ASICs), Application Specific Instruction-set Processor (ASIP), and Digital Signal Processors (DSPs).

### Biosensor

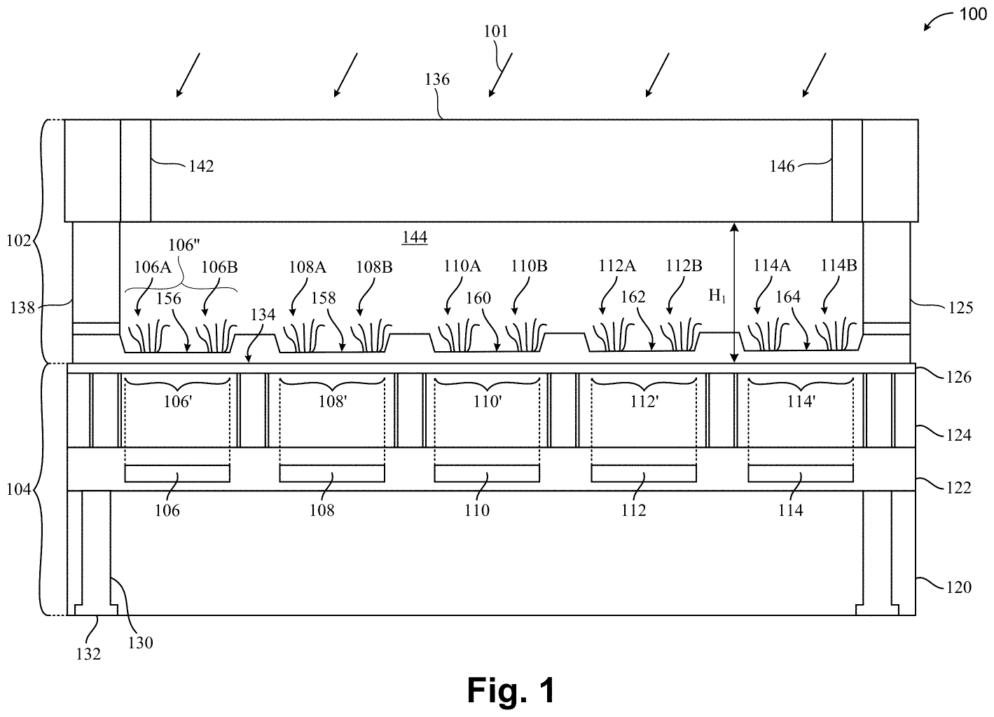

FIG. 1 illustrates a cross-section of a biosensor 100 that can be used in various embodiments. Biosensor 100 has pixel areas 106′, 108′, 110′, 112′, and 114′ that can each hold more than one cluster during a base calling cycle (e.g., 2 clusters per pixel area). As shown, the biosensor 100 may include a flow cell 102 that is mounted onto a sampling device 104. In the illustrated embodiment, the flow cell 102 is affixed directly to the sampling device 104. However, in alternative embodiments, the flow cell 102 may be removably coupled to the sampling device 104. The sampling device 104 has a sample surface 134 that may be functionalized (e.g., chemically or physically modified in a suitable manner for conducting the desired reactions). For example, the sample surface 134 may be functionalized and may include a plurality of pixel areas 106′, 108′, 110′, 112′, and 114′ that can each hold more than one cluster during a base calling cycle (e.g., each having a corresponding cluster pair 106A, 106B; 108A, 108B; 110A, 110B; 112A, 112B; and 114A, 114B immobilized thereto). Each pixel area is associated with a corresponding sensor (or pixel or photodiode) 106, 108, 110, 112, and 114, such that light received by the pixel area is captured by the corresponding sensor. A pixel area 106′ can be also associated with a corresponding reaction site 106″ on the sample surface 134 that holds a cluster pair, such that light emitted from the reaction site 106″ is received by the pixel area 106′ and captured by the corresponding sensor 106. As a result of this sensing structure, in the case in which two or more clusters are present in a pixel area of a particular sensor during a base calling cycle (e.g., each having a corresponding cluster pair), the pixel signal in that base calling cycle carries information based on all of the two or more clusters. As a result, signal processing as described herein is used to distinguish each cluster, where there are more clusters than pixel signals in a given sampling event of a particular base calling cycle.

In the illustrated embodiment, the flow cell 102 includes sidewalls 138, 125, and a flow cover 136 that is supported by the sidewalls 138, 125. The sidewalls 138, 125 are coupled to the sample surface 134 and extend between the flow cover 136 and the sidewalls 138, 125. In some embodiments, the sidewalls 138, 125 are formed from a curable adhesive layer that bonds the flow cover 136 to the sampling device 104.

The sidewalls 138, 125 are sized and shaped so that a flow channel 144 exists between the flow cover 136 and the sampling device 104. The flow cover 136 may include a material that is transparent to excitation light 101 propagating from an exterior of the biosensor 100 into the flow channel 144. In an example, the excitation light 101 approaches the flow cover 136 at a non-orthogonal angle.

Also shown, the flow cover 136 may include inlet and outlet ports 142, 146 that are configured to fluidically engage other ports (not shown). For example, the other ports may be from the cartridge or the workstation. The flow channel 144 is sized and shaped to direct a fluid along the sample surface 134. A height H1 and other dimensions of the flow channel 144 may be configured to maintain a substantially even flow of a fluid along the sample surface 134. The dimensions of the flow channel 144 may also be configured to control bubble formation.

By way of example, the flow cover 136 (or the flow cell 102) may comprise a transparent material, such as glass or plastic. The flow cover 136 may constitute a substantially rectangular block having a planar exterior surface and a planar inner surface that defines the flow channel 144. The block may be mounted onto the sidewalls 138, 125. Alternatively, the flow cell 102 may be etched to define the flow cover 136 and the sidewalls 138, 125. For example, a recess may be etched into the transparent material. When the etched material is mounted to the sampling device 104, the recess may become the flow channel 144.

The sampling device 104 may be similar to, for example, an integrated circuit comprising a plurality of stacked substrate layers 120-126. The substrate layers 120-126 may include a base substrate 120, a solid-state imager 122 (e.g., CMOS image sensor), a filter or light-management layer 124, and a passivation layer 126. It should be noted that the above is only illustrative and that other embodiments may include fewer or additional layers. Moreover, each of the substrate layers 120-126 may include a plurality of sub-layers. The sampling device 104 may be manufactured using processes that are similar to those used in manufacturing integrated circuits, such as CMOS image sensors and CCDs. For example, the substrate layers 120-126 or portions thereof may be grown, deposited, etched, and the like to form the sampling device 104.

The passivation layer 126 is configured to shield the filter layer 124 from the fluidic environment of the flow channel 144. In some cases, the passivation layer 126 is also configured to provide a solid surface (i.e., the sample surface 134) that permits biomolecules or other analytes-of-interest to be immobilized thereon. For example, each of the reaction sites may include a cluster of biomolecules that are immobilized to the sample surface 134. Thus, the passivation layer 126 may be formed from a material that permits the reaction sites to be immobilized thereto. The passivation layer 126 may also comprise a material that is at least transparent to a desired fluorescent light. By way of example, the passivation layer 126 may include silicon nitride (Si₂N₄) and/or silica (SiO₂) However, other suitable material(s) may be used. In the illustrated embodiment, the passivation layer 126 may be substantially planar. However, in alternative embodiments, the passivation layer 126 may include recesses, such as pits, wells, grooves, and the like. In the illustrated embodiment, the passivation layer 126 has a thickness that is about 150-200 nm and, more particularly, about 170 nm.

The filter layer 124 may include various features that affect the transmission of light. In some embodiments, the filter layer 124 can perform multiple functions. For instance, the filter layer 124 may be configured to (a) filter unwanted light signals, such as light signals from an excitation light source; (b) direct emission signals from the reaction sites toward corresponding sensors 106, 108, 110, 112, and 114 that are configured to detect the emission signals from the reaction sites; or (c) block or prevent detection of unwanted emission signals from adjacent reaction sites. As such, the filter layer 124 may also be referred to as a light-management layer. In the illustrated embodiment, the filter layer 124 has a thickness that is about 1-5 μm and, more particularly, about 2-4 μm. In alternative embodiments, the filter layer 124 may include an array of microlenses or other optical components. Each of the microlenses may be configured to direct emission signals from an associated reaction site to a sensor.

In some embodiments, the solid-state imager 122 and the base substrate 120 may be provided together as a previously constructed solid-state imaging device (e.g., CMOS chip). For example, the base substrate 120 may be a wafer of silicon and the solid-state imager 122 may be mounted thereon. The solid-state imager 122 includes a layer of semiconductor material (e.g., silicon) and the sensors 106, 108, 110, 112, and 114. In the illustrated embodiment, the sensors are photodiodes configured to detect light. In other embodiments, the sensors comprise light detectors. The solid-state imager 122 may be manufactured as a single chip through a CMOS-based fabrication processes.

The solid-state imager 122 may include a dense array of sensors 106, 108, 110, 112, and 114 that are configured to detect activity indicative of a desired reaction from within or along the flow channel 144. In some embodiments, each sensor has a pixel area (or detection area) that is about 1-2 square micrometer (μm²). The array can include 500,000 sensors, 5 million sensors, 10 million sensors, or even 120 million sensors. The sensors 106, 108, 110, 112, and 114 can be configured to detect a predetermined wavelength of light that is indicative of the desired reactions.

In some embodiments, the sampling device 104 includes a microcircuit arrangement, such as the microcircuit arrangement described in U.S. Pat. No. 7,595,882, which is incorporated herein by reference in the entirety. More specifically, the sampling device 104 may comprise an integrated circuit having a planar array of the sensors 106, 108, 110, 112, and 114. Circuitry formed within the sampling device 104 may be configured for at least one of signal amplification, digitization, storage, and processing. The circuitry may collect and analyze the detected fluorescent light and generate pixel signals (or detection signals) for communicating detection data to a signal processor. The circuitry may also perform additional analog and/or digital signal processing in the sampling device 104. Sampling device 104 may include conductive vias 130 that perform signal routing (e.g., transmit the pixel signals to the signal processor). The pixel signals may also be transmitted through electrical contacts 132 of the sampling device 104.

The sampling device 104 is discussed in further details with respect to U.S. Nonprovisional patent application Ser. No. 16/874,599, titled “Systems and Devices for Characterization and Performance Analysis of Pixel-Based Sequencing,” filed May 14, 2020 (Attorney Docket No. ILLM 1011-4/IP-1750-US), which is incorporated by reference as if fully set forth herein. The sampling device 104 is not limited to the above constructions or uses as described above. In alternative embodiments, the sampling device 104 may take other forms. For example, the sampling device 104 may comprise a CCD device, such as a CCD camera, that is coupled to a flow cell or is moved to interface with a flow cell having reaction sites therein.

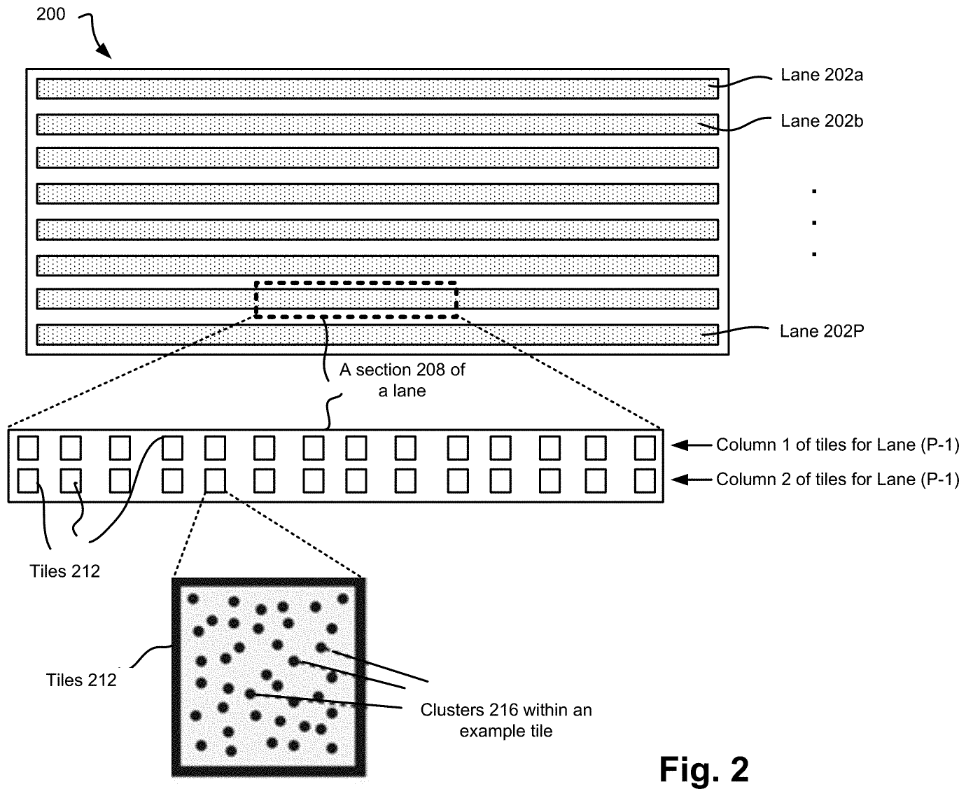

FIG. 2 depicts one implementation of a flow cell 200 that contains clusters in its tiles. The flow cell 200 corresponds to the flow cell 102 of FIG. 1, e.g., without the flow cover 136. Furthermore, the depiction of the flow cell 200 is symbolic in nature, and the flow cell 200 symbolically depicts various lanes and tiles therewithin, without illustrating various other components therewithin. FIG. 2 illustrates a top view of the flow cell 200.

In an embodiment, the flow cell 200 is divided or partitioned in a plurality of lanes, such as lanes 202a, 202b, . . . , 202P, i.e., P number of lanes. In the example of FIG. 2, the flow cell 200 is illustrated to include 8 lanes, i.e., P=8 in this example, although the number of lanes within a flow cell is implementation specific.

In an embodiment, individual lanes 202 are further partitioned into non-overlapping regions called “tiles” 212. For example, FIG. 2 illustrates a magnified view of a section 208 of an example lane. The section 208 is illustrated to comprise a plurality of tiles 212.

In an example, each lane 202 comprises one or more columns of tiles. For example, in FIG. 2, each lane 202 comprises two corresponding columns of tiles 212, as illustrated within the magnified section 208. A number of tiles within each column of tiles within each lane is implementation specific, and in one example, there can be 50 tiles, 60 tiles, 100 tiles, or another appropriate number of tiles in each column of tiles within each lane.

Each tile comprises a corresponding plurality of clusters. During the sequencing procedure, the clusters and their surrounding background on the tiles are imaged. For example, FIG. 2 illustrates example clusters 216 within an example tile.

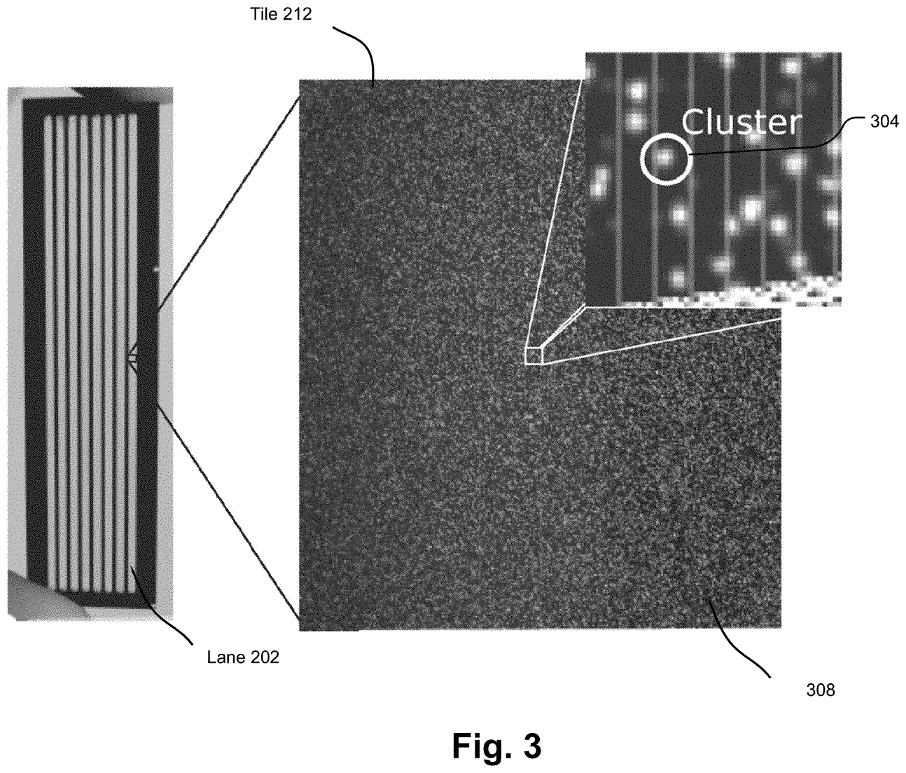

FIG. 3 illustrates an example Illumina GA-IIx™ flow cell with eight lanes, and also illustrates a zoom-in on one tile and its clusters and their surrounding background. For example, there are a hundred tiles per lane in Illumina Genome Analyzer II and sixty-eight tiles per lane in Illumina HiSeq2000. A tile 212 holds hundreds of thousands to millions of clusters. In FIG. 3, an image generated from a tile with clusters shown as bright spots is shown at 308 (e.g., 308 is a magnified image view of a tile), with an example cluster 304 labelled. A cluster 304 comprises approximately one thousand identical copies of a template molecule, though clusters vary in size and shape. The clusters are grown from the template molecule, prior to the sequencing run, by bridge amplification of the input library. The purpose of the amplification and cluster growth is to increase the intensity of the emitted signal since the imaging device cannot reliably sense a single fluorophore. However, the physical distance of the DNA fragments within a cluster 304 is small, so the imaging device perceives the cluster of fragments as a single spot 304.

The clusters and the tiles are discussed in further details with respect to U.S. Nonprovisional patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US).

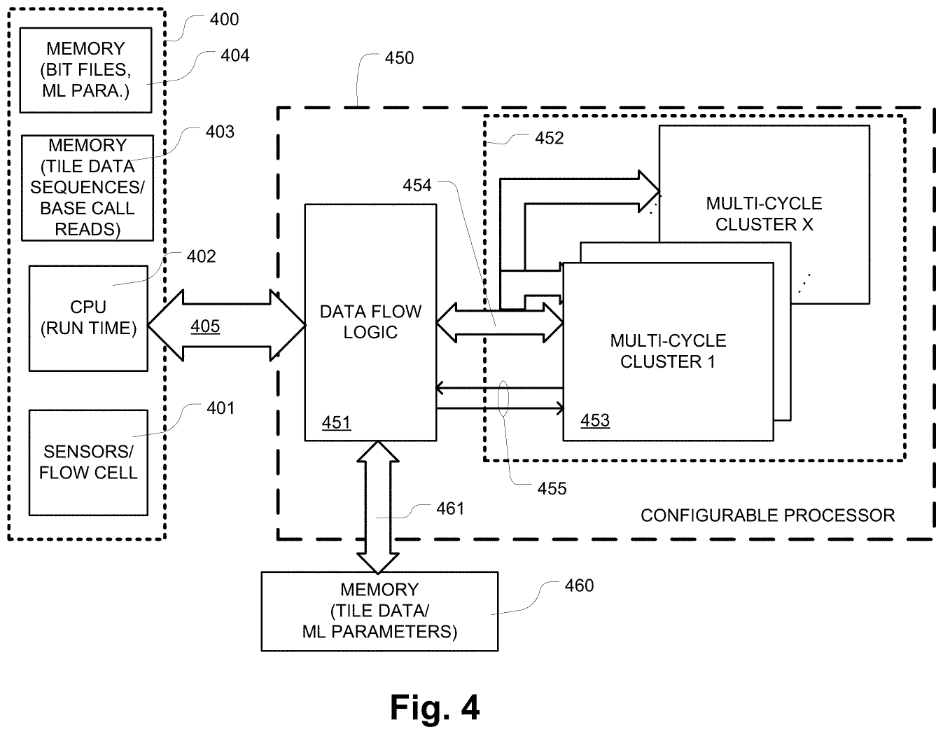

FIG. 4 is a simplified block diagram of the system for analysis of sensor data from a sequencing system, such as base call sensor outputs (e.g., see FIG. 1). In the example of FIG. 4, the system includes a sequencing machine 400 and a configurable processor 450. The configurable processor 450 can execute a neural network-based base caller and/or a non-neural network-based base caller (which will be discussed herein in further detail) in coordination with a runtime program executed by a host processor, such as a central processing unit (CPU) 402. The sequencing machine 400 comprises base call sensors and flow cell 401 (e.g., discussed with respect to FIGS. 1-3). The flow cell can comprise one or more tiles in which clusters of genetic material are exposed to a sequence of analyte flows used to cause reactions in the clusters to identify the bases in the genetic material, as discussed with respect to FIGS. 1-3. The sensors sense the reactions for each cycle of the sequence in each tile of the flow cell to provide tile data. Examples of this technology are described in more detail below. Genetic sequencing is a data intensive operation, which translates base call sensor data into sequences of base calls for each cluster of genetic material sensed in during a base call operation.

The system in this example includes the CPU 402 which executes a runtime program to coordinate the base call operations, memory 403 to store sequences of arrays of tile data, base call reads produced by the base calling operation, and other information used in the base call operations. Also, in this illustration the system includes memory 404 to store a configuration file (or files), such as FPGA bit files, and model parameters for the neural network used to configure and reconfigure the configurable processor 450 and execute the neural network. The sequencing machine 400 can include a program for configuring a configurable processor and in some embodiments a reconfigurable processor to execute the neural network.

The sequencing machine 400 is coupled by a bus 405 to the configurable processor 450. The bus 405 can be implemented using a high throughput technology, such as in one example bus technology compatible with the PCIe standards (Peripheral Component Interconnect Express) currently maintained and developed by the PCI-SIG (PCI Special Interest Group). Also, in this example, a memory 460 is coupled to the configurable processor 450 by bus 461. The memory 460 can be on-board memory, disposed on a circuit board with the configurable processor 450. The memory 460 is used for high-speed access by the configurable processor 450 of working data used in the base call operation. The bus 461 can also be implemented using a high throughput technology, such as bus technology compatible with the PCIe standards

Configurable processors, including Field Programmable Gate Arrays (FPGAs), Coarse Grained Reconfigurable Arrays (CGRAs), and other configurable and reconfigurable devices, can be configured to implement a variety of functions more efficiently or faster than might be achieved using a general-purpose processor executing a computer program. Configuration of configurable processors involves compiling a functional description to produce a configuration file, referred to sometimes as a bitstream or bit file, and distributing the configuration file to the configurable elements on the processor.

The configuration file defines the logic functions to be executed by the configurable processor, by configuring the circuit to set data flow patterns, use of distributed memory and other on-chip memory resources, lookup table contents, operations of configurable logic blocks and configurable execution units like multiply-and-accumulate units, configurable interconnects and other elements of the configurable array. A configurable processor is reconfigurable if the configuration file may be changed in the field, by changing the loaded configuration file. For example, the configuration file may be stored in volatile SRAM elements, in non-volatile read-write memory elements, and in combinations of the same, distributed among the array of configurable elements on the configurable or reconfigurable processor. A variety of commercially available configurable processors are suitable for use in a base calling operation as described herein. Examples include commercially available products such as Xilinx Alveo™ U200, Xilinx Alveo™ U250, Xilinx Alveo™ U280, Intel/Altera Stratix™ GX2800, Intel/Altera Stratix™ GX2800, and Intel Stratix™ GX10M. In some examples, a host CPU can be implemented on the same integrated circuit as the configurable processor.

Embodiments described herein implement the multi-cycle neural network using a configurable processor 450. The configuration file for a configurable processor can be implemented by specifying the logic functions to be executed using a high-level description language (HDL) or a register transfer level (RTL) language specification. The specification can be compiled using the resources designed for the selected configurable processor to generate the configuration file. The same or similar specification can be compiled for the purposes of generating a design for an application-specific integrated circuit which may not be a configurable processor.

Alternatives for the configurable processor, in all embodiments described herein, therefore include a configured processor comprising an application specific ASIC or special purpose integrated circuit or set of integrated circuits, or a system-on-a-chip SOC device, configured to execute a neural network based base call operation as described herein.

In general, configurable processors and configured processors described herein, as configured to execute runs of a neural network, are referred to herein as neural network processors. In another example, configurable processors and configured processors described herein, as configured to execute runs of a non-neural network based base caller, are referred to herein as non-neural network processors. In general, the configurable processors and configured processors can be used to implement one or both a neural network based base caller and a non-neural network based base caller, as will be discussed herein later.

The configurable processor 450 is configured in this example by a configuration file loaded using a program executed by the CPU 402, or by other sources, which configures the array of configurable elements on the configurable processor 454 to execute the base call function. In this example, the configuration includes data flow logic 451 which is coupled to the buses 405 and 461 and executes functions for distributing data and control parameters among the elements used in the base call operation.

Also, the configurable processor 450 is configured with base call execution logic 452 to execute a multi-cycle neural network. The logic 452 comprises a plurality of multi-cycle execution clusters (e.g., 453) which, in this example, includes multi-cycle cluster 1 through multi-cycle cluster X. The number of multi-cycle clusters can be selected according to a trade-off involving the desired throughput of the operation, and the available resources on the configurable processor.

The multi-cycle clusters are coupled to the data flow logic 451 by data flow paths 454 implemented using configurable interconnect and memory resources on the configurable processor. Also, the multi-cycle clusters are coupled to the data flow logic 451 by control paths 455 implemented using configurable interconnect and memory resources for example on the configurable processor, which provide control signals indicating available clusters, readiness to provide input units for execution of a run of the neural network to the available clusters, readiness to provide trained parameters for the neural network, readiness to provide output patches of base call classification data, and other control data used for execution of the neural network.

The configurable processor is configured to execute runs of a multi-cycle neural network using trained parameters to produce classification data for sensing cycles of the base flow operation. A run of the neural network is executed to produce classification data for a subject sensing cycle of the base call operation. A run of the neural network operates on a sequence including a number N of arrays of tile data from respective sensing cycles of N sensing cycles, where the N sensing cycles provide sensor data for different base call operations for one base position per operation in time sequence in the examples described herein. Optionally, some of the N sensing cycles can be out of sequence if the needed according to a particular neural network model being executed. The number N can be any number greater than one. In some examples described herein, sensing cycles of the N sensing cycles represent a set of sensing cycles for at least one sensing cycle preceding the subject sensing cycle and at least one sensing cycle following the subject cycle in time sequence. Examples are described herein in which the number N is an integer equal to or greater than five.

The data flow logic 451 is configured to move tile data and at least some trained parameters of the model from the memory 460 to the configurable processor for runs of the neural network, using input units for a given run including tile data for spatially aligned patches of the N arrays. The input units can be moved by direct memory access operations in one DMA operation, or in smaller units moved during available time slots in coordination with the execution of the neural network deployed.

Tile data for a sensing cycle as described herein can comprise an array of sensor data having one or more features. For example, the sensor data can comprise two images which are analyzed to identify one of four bases at a base position in a genetic sequence of DNA, RNA, or other genetic material. The tile data can also include metadata about the images and the sensors. For example, in embodiments of the base calling operation, the tile data can comprise information about alignment of the images with the clusters such as distance from center information indicating the distance of each pixel in the array of sensor data from the center of a cluster of genetic material on the tile.

During execution of the multi-cycle neural network as described below, tile data can also include data produced during execution of the multi-cycle neural network, referred to as intermediate data, which can be reused rather than recomputed during a run of the multi-cycle neural network. For example, during execution of the multi-cycle neural network, the data flow logic can write intermediate data to the memory 460 in place of the sensor data for a given patch of an array of tile data. Embodiments like this are described in more detail below.

As illustrated, a system is described for analysis of base call sensor output, comprising memory (e.g., 460) accessible by the runtime program storing tile data including sensor data for a tile from sensing cycles of a base calling operation. Also, the system includes a neural network processor, such as configurable processor 450 having access to the memory. The neural network processor is configured to execute runs of a neural network using trained parameters to produce classification data for sensing cycles. As described herein, a run of the neural network is operating on a sequence of N arrays of tile data from respective sensing cycles of N sensing cycles, including a subject cycle, to produce the classification data for the subject cycle. The data flow logic 451 is provided to move tile data and the trained parameters from the memory to the neural network processor for runs of the neural network using input units including data for spatially aligned patches of the N arrays from respective sensing cycles of N sensing cycles.

Also, a system is described in which the neural network processor has access to the memory, and includes a plurality of execution clusters, the execution logic clusters in the plurality of execution clusters configured to execute a neural network. The data flow logic has access to the memory and to execution clusters in the plurality of execution clusters, to provide input units of tile data to available execution clusters in the plurality of execution clusters, the input units including a number N of spatially aligned patches of arrays of tile data from respective sensing cycles, including a subject sensing cycle, and to cause the execution clusters to apply the N spatially aligned patches to the neural network to produce output patches of classification data for the spatially aligned patch of the subject sensing cycle, where N is greater than 1.

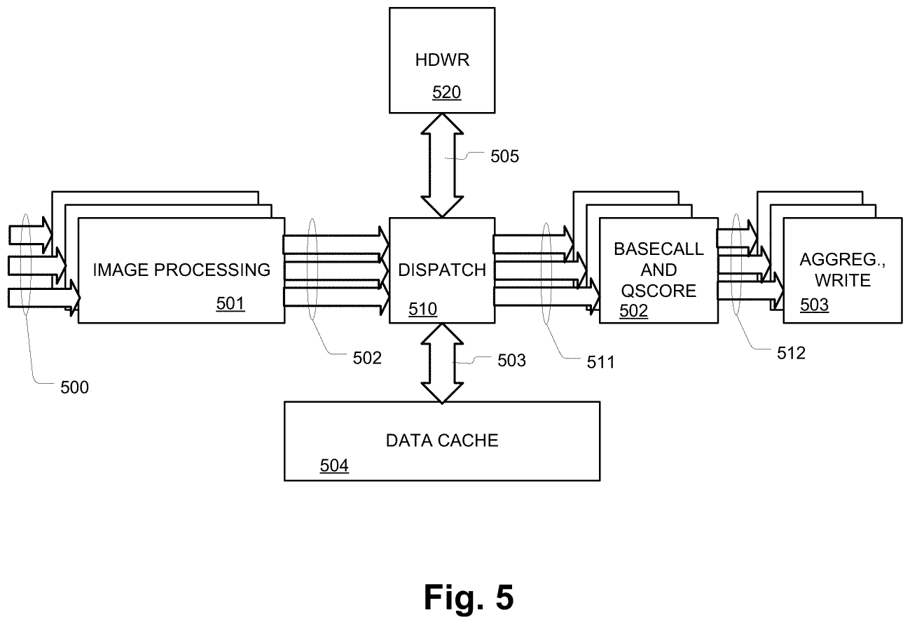

FIG. 5 is a simplified diagram showing aspects of the base calling operation, including functions of a runtime program executed by a host processor. In this diagram, the output of image sensors from a flow cell (such as those illustrated in FIGS. 1-2) are provided on lines 500 to image processing threads 501, which can perform processes on images such as resampling, alignment and arrangement in an array of sensor data for the individual tiles, and can be used by processes which calculate a tile cluster mask for each tile in the flow cell, which identifies pixels in the array of sensor data that correspond to clusters of genetic material on the corresponding tile of the flow cell. To compute a cluster mask, one example algorithm is based on a process to detect clusters which are unreliable in the early sequencing cycles using a metric derived from the softmax output, and then the data from those wells/clusters is discarded, and no output data is produced for those clusters. For example, a process can identify clusters with high reliability during the first N1 (e.g., 25) base-calls, and reject the others. Rejected clusters might be polyclonal or very weak intensity or obscured by fiducials. This procedure can be performed on the host CPU. In alternative implementations, this information would potentially be used to identify the necessary clusters of interest to be passed back to the CPU, thereby limiting the storage required for intermediate data.

The outputs of the image processing threads 501 are provided on lines 502 to a dispatch logic 510 in the CPU which routes the arrays of tile data to a data cache 504 on a high-speed bus 503, or on high-speed bus 505 to hardware 520, such as the configurable processor of FIG. 4, according to the state of the base calling operation. The hardware 520 can be multi-cluster neural network processor to execute a neural network based base caller, or can be hardware to execute a non-neural based base caller, as will be discussed herein later.

The hardware 520 returns classification data (e.g., output by the neural network base caller and/or the non-neural network base caller) to the dispatch logic 510, which passes the information to the data cache 504, or on lines 511 to threads 502 that perform base call and quality score computations using the classification data, and can arrange the data in standard formats for base call reads. The outputs of the threads 502 that perform base calling and quality score computations are provided on lines 512 to threads 503 that aggregate the base call reads, perform other operations such as data compression, and write the resulting base call outputs to specified destinations for utilization by the customers.

In some embodiments, the host can include threads (not shown) that perform final processing of the output of the hardware 520 in support of the neural network. For example, the hardware 520 can provide outputs of classification data from a final layer of the multi-cluster neural network. The host processor can execute an output activation function, such as a softmax function, over the classification data to configure the data for use by the base call and quality score threads 502. Also, the host processor can execute input operations (not shown), such as resampling, batch normalization or other adjustments of the tile data prior to input to the hardware 520.

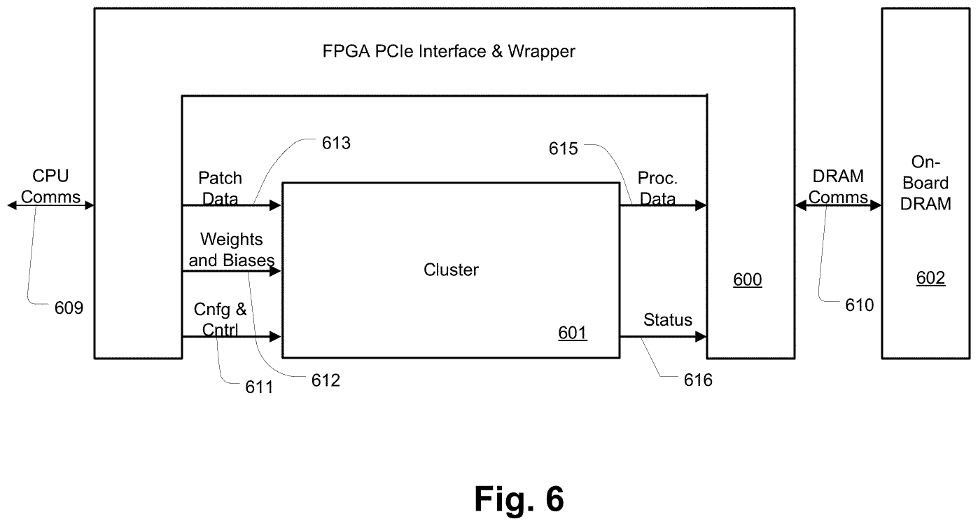

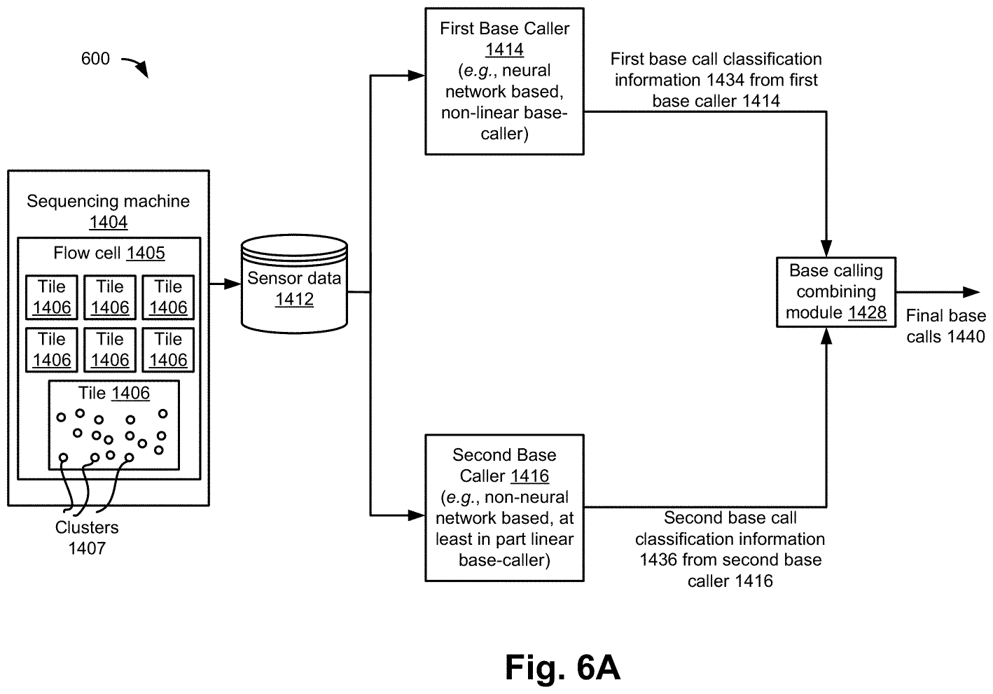

FIG. 6 is a simplified diagram of a configuration of a configurable processor such as that of FIG. 4. In FIG. 6, the configurable processor comprises in FPGA with a plurality of high speed PCIe interfaces. The FPGA is configured with a wrapper 600 which comprises the data flow logic described with reference to FIG. 1. The wrapper 600 manages the interface and coordination with a runtime program in the CPU across the CPU communication link 609 and manages communication with the on-board DRAM 602 (e.g., memory 460) via DRAM communication link 610. The data flow logic in the wrapper 600 provides patch data retrieved by traversing the arrays of tile data on the on-board DRAM 602 for the number N cycles to a cluster 601 and retrieves process data 615 from the cluster 601 for delivery back to the on-board DRAM 602. The wrapper 600 also manages transfer of data between the on-board DRAM 602 and host memory, for both the input arrays of tile data, and for the output patches of classification data. The wrapper transfers patch data on line 613 to the allocated cluster 601. The wrapper provides trained parameters, such as weights and biases on line 612 to the cluster 601 retrieved from the on-board DRAM 602. The wrapper provides configuration and control data on line 611 to the cluster 601 provided from, or generated in response to, the runtime program on the host via the CPU communication link 609. The cluster can also provide status signals on line 616 to the wrapper 600, which are used in cooperation with control signals from the host to manage traversal of the arrays of tile data to provide spatially aligned patch data, and to execute the multi-cycle neural network for base calling and/or operations for non-neural network based base calling, over the patch data using the resources of the cluster 601.

As mentioned above, there can be multiple clusters on a single configurable processor managed by the wrapper 600 configured for executing on corresponding ones of multiple patches of the tile data. Each cluster can be configured to provide classification data for base calls in a subject sensing cycle using the tile data of multiple sensing cycles described herein.

In examples of the system, model data, including kernel data like filter weights and biases can be sent from the host CPU to the configurable processor, so that the model can be updated as a function of cycle number. A base calling operation can comprise, for a representative example, on the order of hundreds of sensing cycles. Base calling operation can include paired end reads in some embodiments. For example, the model trained parameters may be updated once every 20 cycles (or other number of cycles), or according to update patterns implemented for particular systems. In some embodiments including paired end reads in which a sequence for a given string in a genetic cluster on a tile includes a first part extending from a first end down (or up) the string, and a second part extending from a second end up (or down) the string, the trained parameters can be updated on the transition from the first part to the second part.

In some examples, image data for multiple cycles of sensing data for a tile can be sent from the CPU to the wrapper 600. The wrapper 600 can optionally do some pre-processing and transformation of the sensing data and write the information to the on-board DRAM 602. The input tile data for each sensing cycle can include arrays of sensor data including on the order of 4000×3000 pixels per sensing cycle per tile or more, with two features representing colors of two images of the tile, and one or two bytes per feature per pixel. For an embodiment in which the number N is three sensing cycles to be used in each run of the multi-cycle neural network, the array of tile data for each run of the multi-cycle neural network can consume on the order of hundreds of megabytes per tile. In some embodiments of the system, the tile data also includes an array of DFC data, stored once per tile, or other type of metadata about the sensor data and the tiles.

In operation, when a multi-cycle cluster is available, the wrapper allocates a patch to the cluster. The wrapper fetches a next patch of tile data in the traversal of the tile and sends it to the allocated cluster along with appropriate control and configuration information. The cluster can be configured with enough memory on the configurable processor to hold a patch of data including patches from multiple cycles in some systems, that is being worked on in place, and a patch of data that is to be worked on when the current patch of processing is finished using a ping-pong buffer technique or raster scanning technique in various embodiments.

When an allocated cluster completes its run of the neural network for the current patch and produces an output patch, it will signal the wrapper. The wrapper will read the output patch from the allocated cluster, or alternatively the allocated cluster will push the data out to the wrapper. Then the wrapper will assemble output patches for the processed tile in the DRAM 602. When the processing of the entire tile has been completed, and the output patches of data transferred to the DRAM, the wrapper sends the processed output array for the tile back to the host/CPU in a specified format. In some embodiments, the on-board DRAM 602 is managed by memory management logic in the wrapper 600. The runtime program can control the sequencing operations to complete analysis of all the arrays of tile data for all the cycles in the run in a continuous flow to provide real time analysis.

### Multiple Base Callers

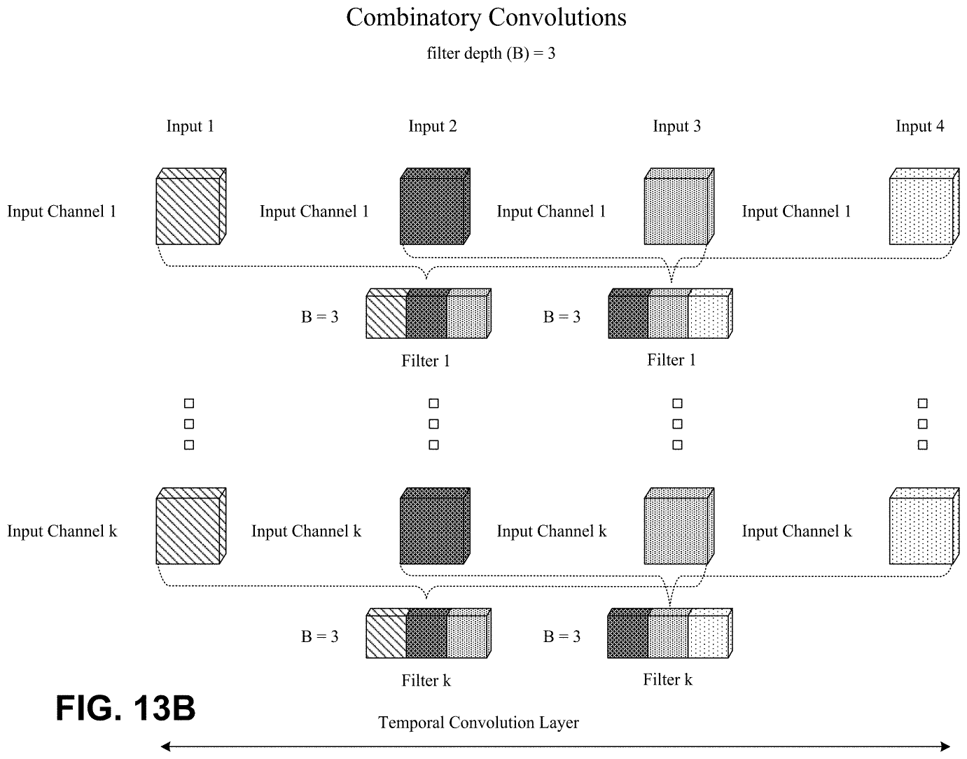

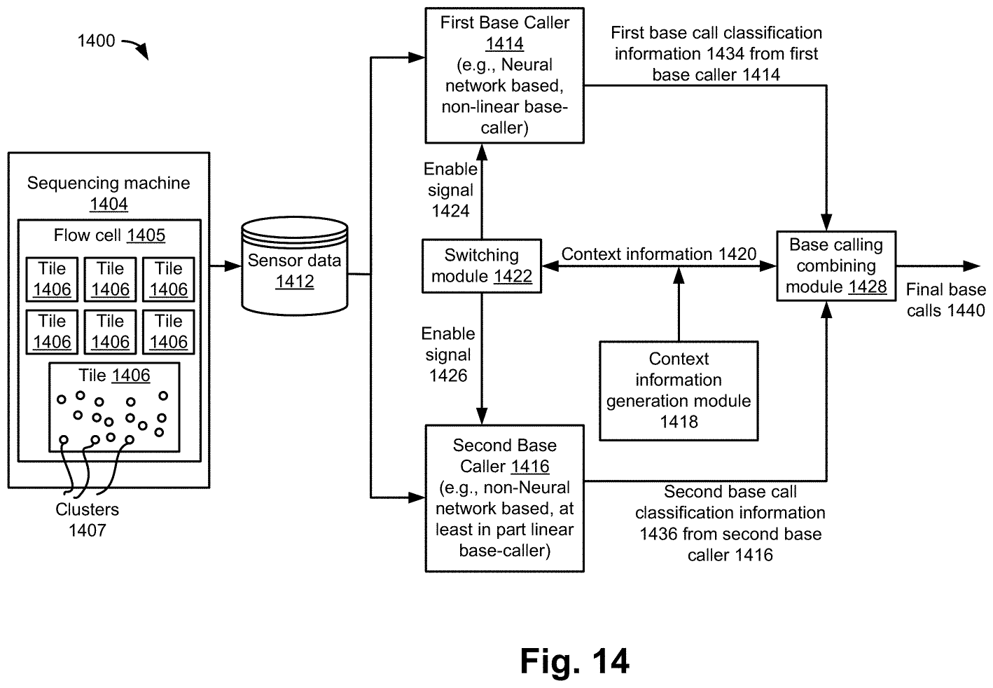

FIG. 6A illustrates a system 600 that employs two or more base callers for base calling operations on the raw images (i.e., sensor data) output by a biosensor. For example, the system 600 includes a sequencing machine 1404, such as the sequencing machine discussed with respect to FIG. 1 (and also discussed with respect to FIG. 14 herein later). The sequencing machine 1404 includes a flow cell 1405, such as the flow cell discussed with respect to FIGS. 1-3. The flow cell 1405 includes a plurality of tiles 1406, and each tile 1406 includes a plurality of clusters 1407 (example clusters of a single tile is illustrated in FIG. 6A), e.g., as discussed with respect to FIGS. 2 and 3. As discussed with respect to FIGS. 4-6, sensor data 1412 comprising raw images from the tiles 1406 are output by the sequencing machine 1404.

In an embodiment, the system 600 comprises two or more base callers, such as a first base caller 1414 and a second base caller 1416. Although two base callers are illustrated in the figure, in an example, more than two base callers may be present in the system 600.

Each base caller of FIG. 6A outputs corresponding base call classification information. For example, the first base caller 1414 outputs first base call classification information 1434, and the second base caller 1416 outputs second base call classification information 1436. A base calling combining module 1428 generates final base calls 1440, based on one or both the first base call classification information 1434 and/or the second base call classification information 1436.

In an example, the first base caller 1414 is a neural-network based base-caller. For example, the first base caller 1414 is a non-linear system employing one or more neural network models for base calling, as will be discussed herein later. The first base caller 1414 is also referred to herein as a DeepRTA (Deep Real Time Analysis) base caller or Deep Neural Network base caller.

In an example, the second base caller 1416 is a non-neural network based base-caller. For example, the second base caller 1416 is at least in part a linear system used for base calling. For example, the second base caller 1416 does not employ a neural network for base calling (or uses a smaller neural network model for base calling, compared to a larger neural network model used by the first base caller 1414), as will be discussed herein later. The second base caller 1416 is also referred to herein as an RTA (Real Time Analysis) base caller.

Examples of a DeepRTA (or Deep Neural Network) base caller and RTA base caller are discussed in U.S. Non-Provisional patent application Ser. No. 16/826,126, entitled “Artificial Intelligence-Based Base Calling,” filed 20 Mar. 2020 (Attorney Docket No. ILLM 1008-18/IP-1744-US), which is incorporated by reference for all purposes as if fully set forth herein.

Further detail of the operation of the system 600 of FIG. 6A, as well as further examples of the first and second base callers 1414 and 1416, will be discussed in further detail herein later, e.g., with respect to FIG. 14.

### Non-Neural Network Based and at Least Partially Linear Base Caller (e.g., the Second Base Caller 1416 of FIGS. 6A and 14)

As discussed with respect to FIG. 6A, the second base caller 1416 is a non-neural network based and at least partially linear base caller. That is, the second base caller 1416 does not employ a neural network for base calling (or uses a smaller neural network model for base calling, compared to a larger neural network model used by the first base caller 1414). An example of the second base caller 1416 is an RTA base caller.

The RTA is a base caller that uses linear intensity extractors to extract features from sequencing images for base calling. The following discussion describes one implementation of intensity extraction and base calling by RTA. In this implementation, RTA performs a template generation step to produce a template image that identifies locations of clusters on a tile using sequencing images from some number of initial sequencing cycles called template cycles. The template image is used as a reference for subsequent registration and intensity extraction steps. The template image is generated by detecting and merging bright spots in each sequencing image of the template cycles, which in turn involves sharpening a sequencing image (e.g., using the Laplacian convolution), determining an “on” threshold by a spatially segregated Otsu approach, and subsequent five-pixel local maximum detection with subpixel location interpolation. In another example, locations of clusters on a tile are identified using fiducial markers. A solid support upon which a biological specimen is imaged can include such fiducial markers, to facilitate determination of the orientation of the specimen or the image thereof in relation to probes that are attached to the solid support. Exemplary fiducials include, but are not limited to beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in U.S. Patent Publication No. 2002/0150909, which is incorporated herein by reference.

RTA then registers a current sequencing image against the template image. This is achieved by using image correlation to align the current sequencing image to the template image on a sub-region, or by using non-linear transformations (e.g., a full six-parameter linear affine transformation).

RTA generates a color matrix to correct cross-talk between color channels of the sequencing images. RTA implements empirical phasing correction to compensate noise in the sequencing images caused by phase errors.

After different corrections are applied to the sequencing images, RTA extracts signal intensities for each spot location in the sequencing images. For example, for a given spot location, signal intensity may be extracted by determining a weighted average of the intensity of the pixels in a spot location. For example, a weighted average of the center pixel and neighboring pixels may be performed using bilinear or bicubic interpolation. In some implementations, each spot location in the image may comprise a few pixels (e.g., 1-5 pixels).

RTA then spatially normalizes the extracted signal intensities to account for variation in illumination across the sampled imaged. For example, intensity values may be normalized such that a 5th and 95th percentiles have values of 0 and 1, respectively. The normalized signal intensities for the image (e.g., normalized intensities for each channel) may be used to calculate mean chastity for the plurality of spots in the image.

In some implementations, RTA uses an equalizer to maximize the signal-to-noise ratio of the extracted signal intensities. The equalizer can be trained (e.g., using least square estimation, adaptive equalization algorithm) to maximize the signal-to-noise ratio of cluster intensity data in sequencing images. In some implementations, the equalizer is a lookup table (LUT) bank with a plurality of LUTs with subpixel resolution, also referred to as “equalizer filters” or “convolution kernels.” In one implementation, the number of LUTs in the equalizer depends on the number of subpixels into which pixels of the sequencing images can be divided. For example, if the pixels are divisible into n by n subpixels (e.g., 5×5 subpixels), then the equalizer generates n2 LUTs (e.g., 25 LUTs).

In one implementation of training the equalizer, data from the sequencing images is binned by well subpixel location. For example, for a 5×5 LUT, 1/25th of the wells have a center that is in bin (1,1) (e.g., the upper left corner of a sensor pixel), 1/25th of the wells are in bin (1,2), and so on. In one implementation, the equalizer coefficients for each bin are determined using least squares estimation on the subset of data from the wells corresponding to the respective bins. This way the resulting estimated equalizer coefficients are different for each bin.

Each LUT/equalizer filter/convolution kernel has a plurality of coefficients that are learned from the training. In one implementation, the number of coefficients in a LUT corresponds to the number of pixels that are used for base calling a cluster. For example, if a local grid of pixels (image or pixel patch) that is used to base call a cluster is of size p×p (e.g., 9×9 pixel patch), then each LUT has p2 coefficients (e.g., 81 coefficients).

In one implementation, the training produces equalizer coefficients that are configured to mix/combine intensity values of pixels that depict intensity emissions from a target cluster being base called and intensity emissions from one or more adjacent clusters in a manner that maximizes the signal-to-noise ratio. The signal maximized in the signal-to-noise ratio is the intensity emissions from the target cluster, and the noise minimized in the signal-to-noise ratio is the intensity emissions from the adjacent clusters, i.e., spatial crosstalk, plus some random noise (e.g., to account for background intensity emissions). The equalizer coefficients are used as weights and the mixing/combining includes executing element-wise multiplication between the equalizer coefficients and the intensity values of the pixels to calculate a weighted sum of the intensity values of the pixels, i.e., a convolution operation.

RTA then performs base calling by fitting a mathematical model to the optimized intensity data. Suitable mathematical models that can be used include, for example, a k-means clustering algorithm, a k-means-like clustering algorithm, expectation maximization clustering algorithm, a histogram based method, and the like. Four Gaussian distributions may be fit to the set of two-channel intensity data such that one distribution is applied for each of the four nucleotides represented in the data set. In one particular implementation, an expectation maximization (EM) algorithm may be applied. As a result of the EM algorithm, for each X, Y value (referring to each of the two channel intensities respectively) a value can be generated which represents the likelihood that a certain X, Y intensity value belongs to one of four Gaussian distributions to which the data is fitted. Where four bases give four separate distributions, each X, Y intensity value will also have four associated likelihood values, one for each of the four bases. The maximum of the four likelihood values indicates the base call. For example, if a cluster is “off” in both channels, the base call is G. If the cluster is “off” in one channel and “on” in another channel the base call is either C or T (depending on which channel is on), and if the cluster is “on” in both channels the base call is A.

Additional details about RTA can be found in U.S. Nonprovisional patent application Ser. No. 15/909,437, titled “OPTICAL DISTORTION CORRECTION FOR IMAGED SAMPLES,” filed on Mar. 1, 2018; U.S. Nonprovisional patent application Ser. No. 14/530,299, titled “IMAGE ANALYSIS USEFUL FOR PATTERNED OBJECTS,” filed on Oct. 31, 2014; U.S. Nonprovisional patent application Ser. No. 15/153,953, titled “METHODS AND SYSTEMS FOR ANALYZING IMAGE DATA,” filed on Dec. 3, 2014; U.S. Nonprovisional patent application Ser. No. 13/006,206, titled “DATA PROCESSING SYSTEM AND METHODS,” filed on Jan. 13, 2011; and U.S. Nonprovisional patent application Ser. No. 17/308,035, titled “EQUALIZATION-BASED IMAGE PROCESSING AND SPATIAL CROSSTALK ATTENUATOR,” filed May 4, 2021 (Attorney Docket No. ILLM 1032-2/IP-1991-US), all which are incorporated by reference as if fully set forth herein.

### Neural Network Based and at Least Partially Non-Linear Base Caller (e.g., the First Base Caller 1414 of FIG. 6A)

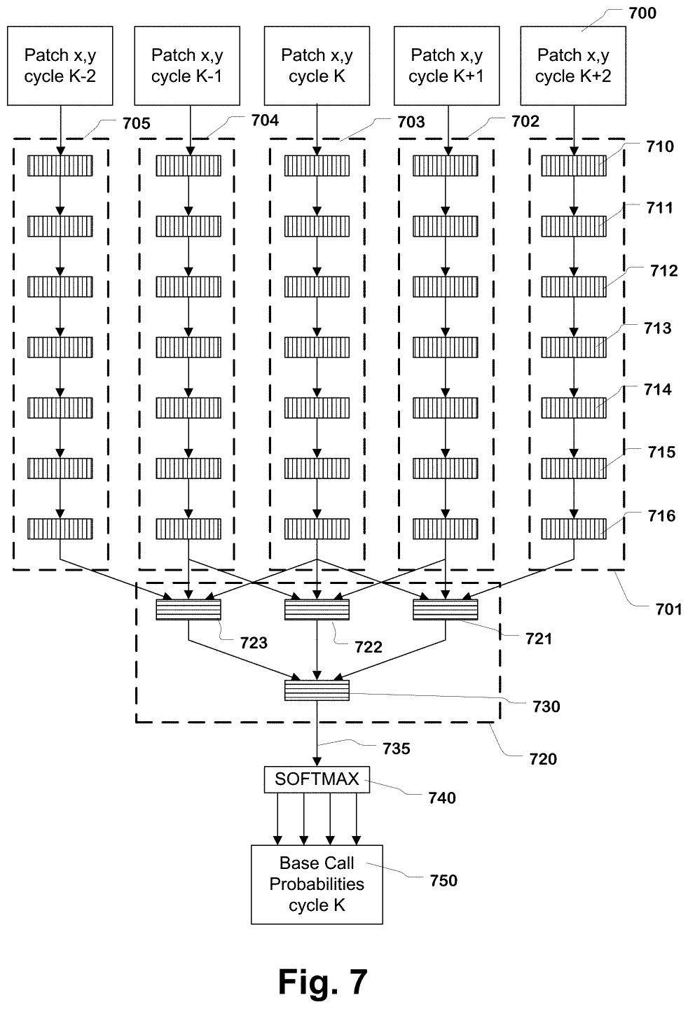

FIGS. 7-13B discusses various examples of the first base caller 1414 of FIG. 6A. For example, FIG. 7 is a diagram of a multi-cycle neural network model which can be executed using the system described herein. The multi-cycle neural network model is an example of the first base caller 1414 of FIG. 6A, although another neural network based model may also be used for the first base caller 1414.

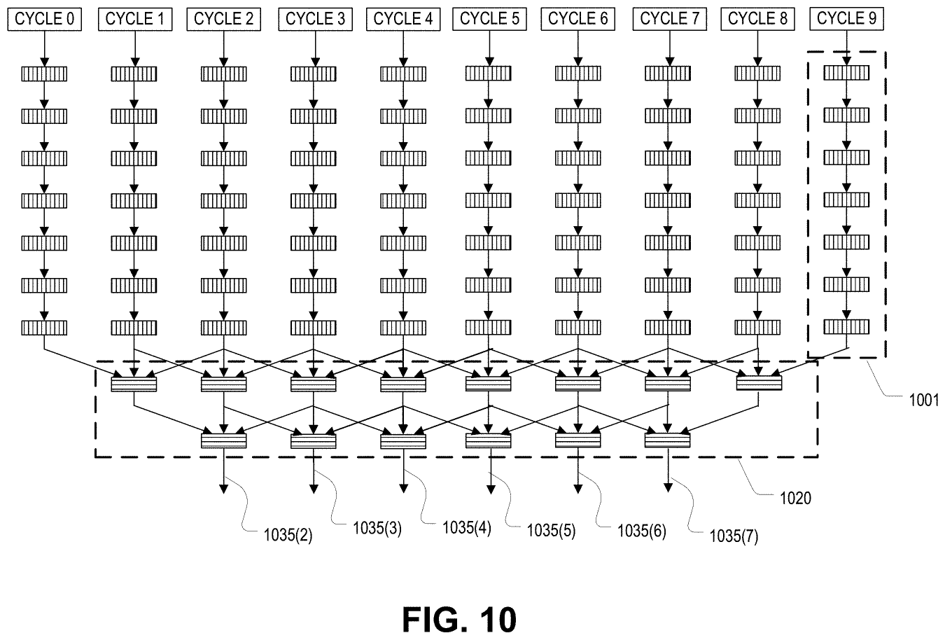

The example shown in FIG. 7 can be referred to as a five-cycle input, one-cycle output neural network. However, note that the five-cycle input, one-cycle output neural network is merely an example, and the neural network can have a different number of inputs (such as six, seven, nine, or another appropriate number). For example, FIG. 10 discussed herein later has a 9-cycle input. Referring again to FIG. 7, the inputs to the multi-cycle neural network model include five spatially aligned patches (e.g., 700) from the tile data arrays of five sensing cycles of a given tile. Spatially aligned patches have the same aligned row and column dimensions (x,y) as other patches in the set, so that the information relates to the same clusters of genetic material on the tile in sequence cycles. In this example, a subject patch is a patch from the array of tile data for cycle K. The set of five spatially aligned patches includes a patch from cycle K−2 preceding the subject patch by two cycles, a patch from cycle K−1 preceding the subject patch by one cycle, a patch from cycle K+1 following the patch from the subject cycle by one cycle, and a patch from cycle K+2 following the patch from the subject cycle by two cycles.

The model includes a segregated stack 701 of layers of the neural network for each of the input patches. Thus, stack 701 receives as input, tile data for the patch from cycle K+2, and is segregated from the stacks 702, 703, 704, and 705 so they do not share input data or intermediate data. In some embodiments, all of the stacks 710-705 can have identical models, and identical trained parameters. In other embodiments, the models and trained parameters may be different in the different stacks. Stack 702 receives as input, tile data for the patch from cycle K+1. Stack 703 receives as input, tile data for the patch from cycle K. Stack 704 receives as input, tile data for the patch from cycle K−1. Stack 705 receives as input, tile data for the patch from cycle K−2. The layers of the segregated stacks each execute a convolution operation of a kernel including a plurality of filters over the input data for the layer. As in the example above, the patch 700 may include three features. The output of the layer 710 may include many more features, such as 10 to 20 features. Likewise, the outputs of each of layers 711 to 716 can include any number of features suitable for a particular implementation. The parameters of the filters are trained parameters for the neural network, such as weights and biases. The output feature set (intermediate data) from each of the stacks 701-705 is provided as input to an inverse hierarchy 720 of temporal combinatorial layers, in which the intermediate data from the multiple cycles is combined. In the example illustrated, the inverse hierarchy 720 includes a first layer including three combinatorial layers 721, 722, 723, each receiving intermediate data from three of the segregated stacks, and a final layer including one combinatorial layer 730 receiving intermediate data from the three temporal layers 721, 722, 723.

The output of the final combinatorial layer 730 is an output patch of classification data for clusters located in the corresponding patch of the tile from cycle K. The output patches can be assembled into an output array classification data for the tile for cycle K. In some embodiments, the output patch may have sizes and dimensions different from the input patches. In some embodiments, the output patch may include pixel-by-pixel data that can be filtered by the host to select cluster data.

The output classification data 735 can then be applied to a softmax function 740 (or other output activation function) optionally executed by the host, or on the configurable processor, depending on the particular implementation. An output function different from softmax could be used (e g., making a base call output parameter according to largest output, then use a learned nonlinear mapping using context/network outputs to give base quality).

Finally, the output of the softmax function 740 can be provided as base call probabilities for cycle K (750) and stored in host memory to be used in subsequent processing. Other systems may use another function for output probability calculation, e.g., another nonlinear model.

The neural network can be implemented using a configurable processor with a plurality of execution clusters so as complete evaluation of one tile cycle within the duration of the time interval, or close to the duration of the time interval, of one sensing cycle, effectively providing the output data in real time. Data flow logic can be configured to distribute input units of tile data and trained parameters to the execution clusters, and to distribute output patches for aggregation in memory.

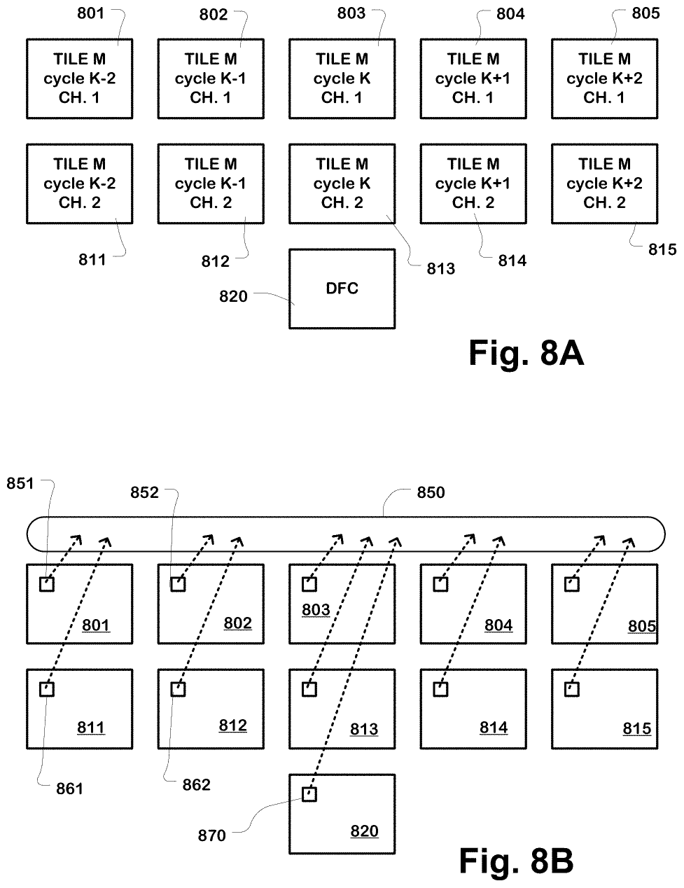

Input units of data for a five-cycle input, one-cycle output neural network like that of FIG. 7 are described with reference to FIGS. 8A and 8B for a base call operation using two-channel sensor data. For example, for a given base in a genetic sequence, the base call operation can execute two flows of analyte and two reactions that generate two channels of signals, such as images, which can be processed to identify which one of four bases is located at a current position in the genetic sequence for each cluster of genetic material. In other systems, a different number of channels of sensing data may be utilized For example, base calling can be performed utilizing one-channel methods and systems. Incorporated materials of U.S. Patent Application Publication No. 2013/0079232 discuss base calling using various number of channels, such as one-channel, two-channels, or four-channels.

FIG. 8A shows arrays of tile data for five cycles for a given tile, tile M, used for the purposes of executing a five-cycle input, one-cycle output neural network. The five-cycle input tile data in this example can be written to the on-board DRAM, or other memory in the system which can be accessed by the data flow logic and, for cycle K−2, includes an array 801 for channel 1 and an array 811 for channel 2, for cycle K−1, an array 802 for channel 1 and an array 812 for channel 2, for cycle K, an array 803 for channel 1 and an array 813 for channel 2, for cycle K+1, an array 804 for channel 1 and an array 814 for channel 2, for cycle K+2, an array 805 for channel 1 and an array 815 for channel 2. Also an array 820 of metadata for the tile can be written once in the memory, in this case a DFC file, included for use as input to the neural network along with each cycle.

Although FIG. 8A discusses two-channel base calling operations, using two channels is merely an example, and base calling can be performed using any other appropriate number of channels. For example, incorporated materials of U.S. Patent Application Publication No. 2013/0079232 discuss base calling using various number of channels, such as one-channel, two-channels, or four-channels, or another appropriate number of channels.

The data flow logic composes input units, which can be understood with reference to FIG. 8B, of tile data that includes spatially aligned patches of the arrays of tile data for each execution cluster configured to execute a run of the neural network over an input patch. An input unit for an allocated execution cluster is composed by the data flow logic by reading spatially aligned patches (e.g., 851, 852, 861, 862, 870) from each of the arrays 801-805, 811, 815, 820 of tile data for the five input cycles, and delivering them via data paths (schematically 850) to memory on the configurable processor configured for use by the allocated execution cluster. The allocated execution cluster executes a run of the five-cycle input/one-cycle output neural network, and delivers an output patch for the subject cycle K of classification data for the same patch of the tile in the subject cycle K.

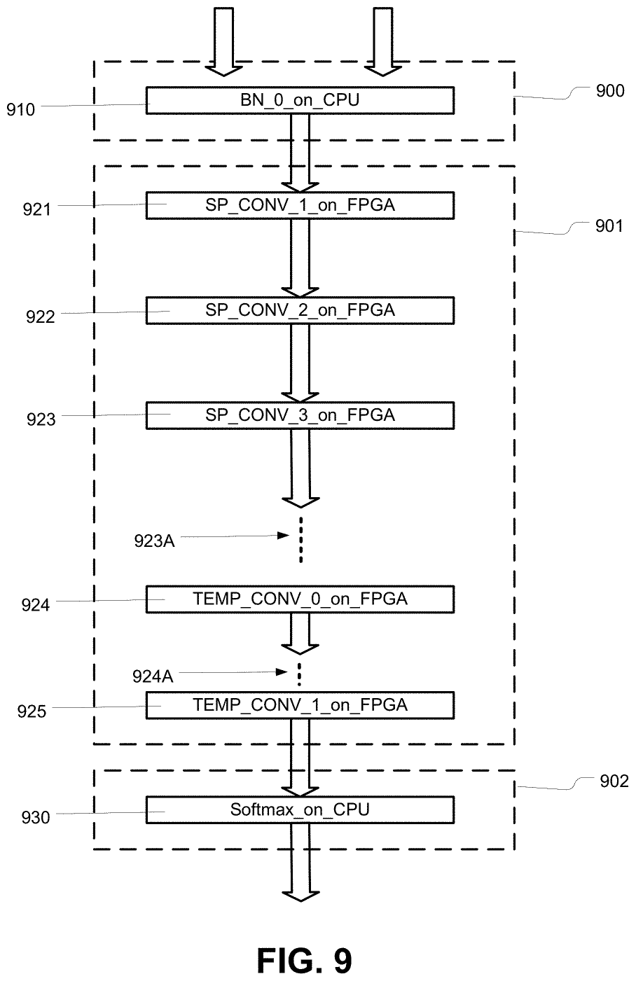

FIG. 9 is a simplified representation of a stack of a neural network usable in a system like that of FIG. 7 (e.g., 701 and 720). In this example, some functions of the neural network (e.g., 900, 902) are executed on the host, and other portions of the neural network (e.g., 901) are executed on the configurable processor.

In an example, a first function can be batch normalization (layer 910) formed on the CPU. However, in another example, batch normalization as a function may be fused into one or more layers, and no separate batch normalization layer may be present.

A number of spatial, segregated convolution layers are executed as a first set of convolution layers of the neural network, as discussed above on the configurable processor. In this example, the first set of convolution layers applies 2D convolutions spatially

As shown in FIG. 9, a first spatial convolution 921 is executed, followed by a second spatial convolution 922, followed by a third spatial convolution 923, and so on for a number L/2 of spatially segregated neural network layers in each stack (L is described with reference to FIG. 7). As indicated at 923A, the number of spatial layers can be any practical number, which for context may range from a few to more than 20 in different embodiments.

For SP_CONV_0, kernel weights are stored for example in a (1,6,6,3,L) structure since there are 3 input channels to this layer.

For other SP_CONV layers, kernel weights are stored for this example in a (1,6,6 L) structure since there are K(=L) inputs and outputs for each of these layers.

The outputs of the stack of spatial layers are provided to temporal layers, including convolution layers 924, 925 executed on the FPGA. Layers 924 and 925 can be convolution layers applying 1D convolutions across cycles. As indicated at 924A, the number of temporal layers can be any practical number, which for context may range from a few to more than 20 in different embodiments.

The first temporal layer, TEMP_CONV_0 layer 924, reduces the number of cycle channels from 5 to 3, as illustrated in FIG. 7. The second temporal layer, layer 925, reduces the number of cycle channels from 3 to 1 as illustrated in FIG. 7, and reduces the number of feature maps to four outputs for each pixel, representing confidence in each base call.

The output of the temporal layers is accumulated in output patches and delivered to the host CPU to apply for example, a softmax function 930, or other function to normalize the base call probabilities.

FIG. 10 illustrates an alternative implementation showing a 10-input, six-output neural network which can be executed for a base calling operation. In this example, tile data for spatially aligned input patches from cycles 0 to 9 are applied to segregated stacks of spatial layers, such as stack 1001 for cycle 9. The outputs of the segregated stacks are applied to an inverse hierarchical arrangement of temporal stacks 1020, having outputs 1035(2) through 1035(7) providing base call classification data for subject cycles 2 through 7.

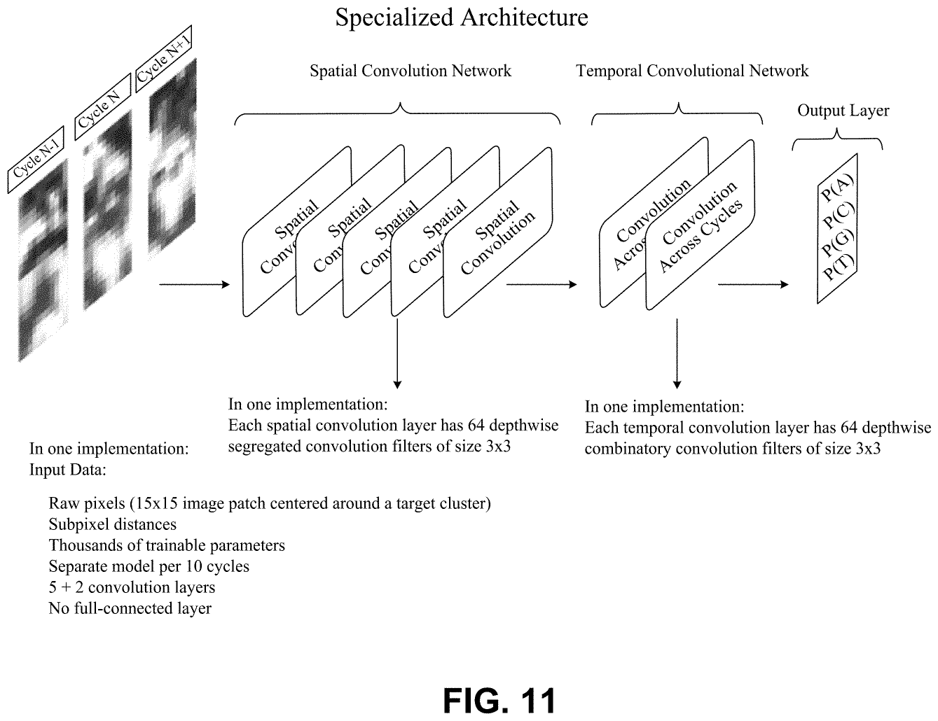

FIG. 11 illustrates one implementation of the specialized architecture of the neural network-based base caller (e.g., FIG. 7) that is used to segregate processing of data for different sequencing cycles. The motivation for using the specialized architecture is described first.

The neural network-based base caller processes data for a current sequencing cycle, one or more preceding sequencing cycles, and one or more successive sequencing cycles. Data for additional sequencing cycles provides sequence-specific context. During training, the neural network-based base caller learns to use the sequence-specific context to improve base calling accuracy. Furthermore, data for pre and post sequencing cycles provides second order contribution of pre-phasing and phasing signals to the current sequencing cycle.

Spatial convolution layers use so-called “segregated convolutions” that operationalize the segregation by independently processing data for each of a plurality of sequencing cycles through a “dedicated, non-shared” sequence of convolutions. The segregated convolutions convolve over data and resulting feature maps of only a given sequencing cycle, i.e., intra-cycle, without convolving over data and resulting feature maps of any other sequencing cycle.

Consider, for example, that the input data comprises (i) current data for a current (time t) sequencing cycle to be base called, (ii) previous data for a previous (time t−1) sequencing cycle, and (iii) next data for a next (time t+1) sequencing cycle. The specialized architecture then initiates three separate data processing pipelines (or convolution pipelines), namely, a current data processing pipeline, a previous data processing pipeline, and a next data processing pipeline. The current data processing pipeline receives as input the current data for the current (time t) sequencing cycle and independently processes it through a plurality of spatial convolution layers to produce a so-called “current spatially convolved representation” as the output of a final spatial convolution layer. The previous data processing pipeline receives as input the previous data for the previous (time t−1) sequencing cycle and independently processes it through the plurality of spatial convolution layers to produce a so-called “previous spatially convolved representation” as the output of the final spatial convolution layer. The next data processing pipeline receives as input the next data for the next (time t+1) sequencing cycle and independently processes it through the plurality of spatial convolution layers to produce a so-called “next spatially convolved representation” as the output of the final spatial convolution layer.

In some implementations, the current pipeline, one or more previous pipeline(s), and one or more next processing pipeline(s) are executed in parallel.

In some implementations, the spatial convolution layers are part of a spatial convolutional network (or subnetwork) within the specialized architecture.

The neural network-based base caller further comprises temporal convolution layers that mix information between sequencing cycles, i.e., inter-cycles. The temporal convolution layers receive their inputs from the spatial convolutional network and operate on the spatially convolved representations produced by the final spatial convolution layer for the respective data processing pipelines.

Temporal convolution layers use so-called “combinatory convolutions” that groupwise convolve over input channels in successive inputs on a sliding window basis. In one implementation, the successive inputs are successive outputs produced by a previous spatial convolution layer or a previous temporal convolution layer.

In some implementations, the temporal convolution layers are part of a temporal convolutional network (or subnetwork) within the specialized architecture. The temporal convolutional network receives its inputs from the spatial convolutional network. In one implementation, a first temporal convolution layer of the temporal convolutional network groupwise combines the spatially convolved representations between the sequencing cycles. In another implementation, subsequent temporal convolution layers of the temporal convolutional network combine successive outputs of previous temporal convolution layers. In an example, a compression logic (or compression network or compression subnetwork or compression layer or squeezing layer) processes outputs of the temporal and/or the spatial convolution network, and generates a compressed representation of the outputs. In one implementation, the compression network comprises a compression convolution layer that reduces the depth dimensionality of feature maps generated by the network.

The output of the final temporal convolution layer (e.g., with or without the compression) is fed to an output layer that produces an output. The output is used to base call one or more clusters at one or more sequencing cycles.

During a forward propagation, the specialized architecture processes information from a plurality of inputs in two stages. In the first stage, segregated convolutions are used to prevent mixing of information between the inputs. In the second stage, combinatory convolutions are used to mix information between the inputs. The results from the second stage are used to make a single inference for the plurality of inputs.

This is different than the batch mode technique where a convolution layer processes multiple inputs in a batch at the same time and makes a corresponding inference for each input in the batch. In contrast, the specialized architecture maps the plurality of inputs to the single inference. The single inference can comprise more than one prediction, such as a classification score for each of the four bases (A, C, T, and G).

In one implementation, the inputs have temporal ordering such that each input is generated at a different time step and has a plurality of input channels. For example, the plurality of inputs can include the following three inputs: a current input generated by a current sequencing cycle at time step (t), a previous input generated by a previous sequencing cycle at time step (t−1), and a next input generated by a next sequencing cycle at time step (t+1). In another implementation, each input is respectively derived from the current, previous, and next inputs by one or more previous convolution layers and includes k feature maps.

In one implementation, each input can include the following five input channels: a red image channel (in red), a red distance channel (in yellow), a green image channel (in green), a green distance channel (in purple), and a scaling channel (in blue). In another implementation, each input can be in blue and violet color channels (or one or more other appropriate color channels), instead of or in addition to red and green channels. In another implementation, each input can be in blue and violet color channels, instead of or in addition to red, green, purple, and/or yellow channels. In another implementation, each input can include k feature maps produced by a previous convolution layer and each feature map is treated as an input channel. In yet another example, each input can have merely one channel, two channels, or another different number of channels. Incorporated materials of U.S. Patent Application Publication No. 2013/0079232 discuss base calling using various number of channels, such as one-channel, two-channels, or four-channels.

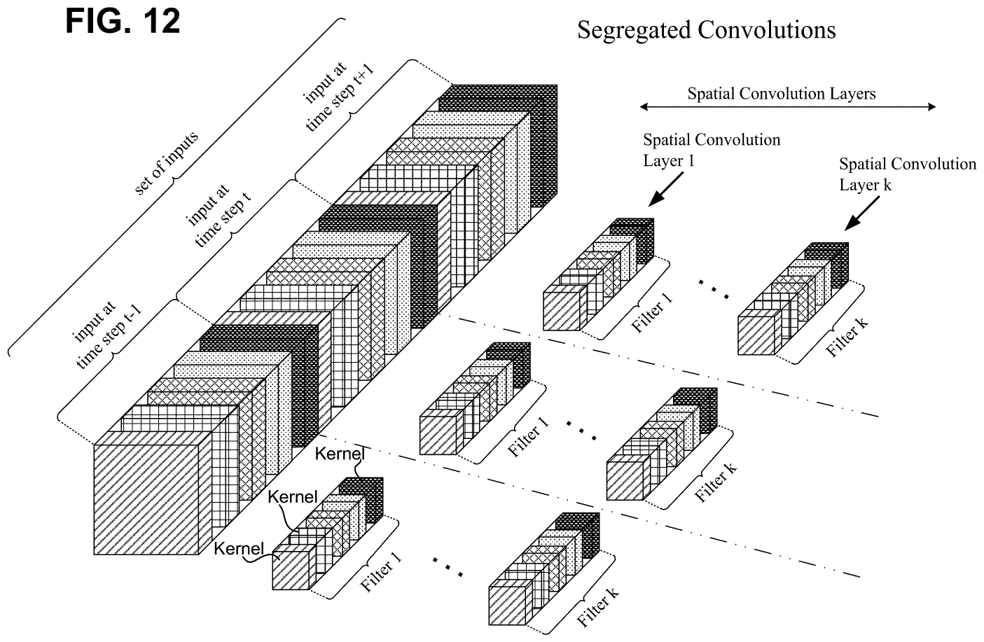

FIG. 12 depicts one implementation of segregated layers, each of which can include convolutions. Segregated convolutions process the plurality of inputs at once by applying a convolution filter to each input in parallel. With the segregated convolutions, the convolution filter combines input channels in a same input and does not combine input channels in different inputs. In one implementation, a same convolution filter is applied to each input in parallel. In another implementation, a different convolution filter is applied to each input in parallel. In some implementations, each spatial convolution layer comprises a bank of k convolution filters, each of which applies to each input in parallel.

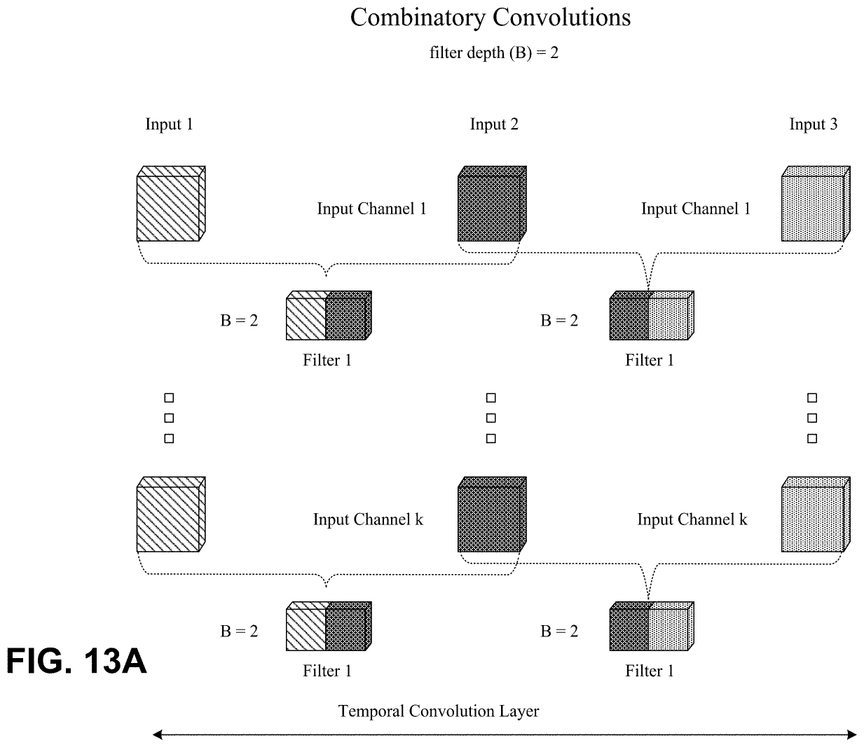

FIG. 13A depicts one implementation of combinatory layers, each of which can include convolutions. FIG. 13B depicts another implementation of the combinatory layers, each of which can include convolutions. Combinatory convolutions mix information between different inputs by grouping corresponding input channels of the different inputs and applying a convolution filter to each group. The grouping of the corresponding input channels and application of the convolution filter occurs on a sliding window basis. In this context, a window spans two or more successive input channels representing, for instance, outputs for two successive sequencing cycles. Since the window is a sliding window, most input channels are used in two or more windows.

In some implementations, the different inputs originate from an output sequence produced by a preceding spatial or temporal convolution layer. In the output sequence, the different inputs are arranged as successive outputs and therefore viewed by a next temporal convolution layer as successive inputs. Then, in the next temporal convolution layer, the combinatory convolutions apply the convolution filter to groups of corresponding input channels in the successive inputs.

In one implementation, the successive inputs have temporal ordering such that a current input is generated by a current sequencing cycle at time step (t), a previous input is generated by a previous sequencing cycle at time step (t−1), and a next input is generated by a next sequencing cycle at time step (t+1). In another implementation, each successive input is respectively derived from the current, previous, and next inputs by one or more previous convolution layers and includes k feature maps.

In one implementation, each input can include the following five input channels: a red image channel (in red), a red distance channel (in yellow), a green image channel (in green), a green distance channel (in purple), and a scaling channel (in blue). In another implementation, an additional input channel can be violet color channel. In another implementation, each input can include k feature maps produced by a previous convolution layer and each feature map is treated as an input channel.

The depth B of the convolution filter is dependent upon the number of successive inputs whose corresponding input channels are groupwise convolved by the convolution filter on a sliding window basis. In other words, the depth B is equal to the number of successive inputs in each sliding window and the group size.

In FIG. 13A, corresponding input channels from two successive inputs are combined in each sliding window, and therefore B=2. In FIG. 13B, corresponding input channels from three successive inputs are combined in each sliding window, and therefore B=3.

In one implementation, the sliding windows share a same convolution filter. In another implementation, a different convolution filter is used for each sliding window. In some implementations, each temporal convolution layer comprises a bank of k convolution filters, each of which applies to the successive inputs on a sliding window basis.

Further detail of FIGS. 4-10, and variations thereof, can be found in co-pending U.S. Nonprovisional patent application Ser. No. 17/176,147, titled “HARDWARE EXECUTION AND ACCELERATION OF ARTIFICIAL INTELLIGENCE-BASED BASE CALLER,” filed Feb. 15, 2021 (Attorney Docket No. ILLM 1020-2/IP-1866-US), which is incorporated by reference as if fully set forth herein.

### Base Calling Using Multiple Base Callers

FIG. 14 illustrates a base calling system 1400 comprising a plurality of base callers, to predict base calls of an unknown analyte comprising base sequences.

Note that the previously discussed FIG. 6A illustrated only some components of the system 1400 of FIG. 14, and FIG. 14 illustrates various other components that were not illustrated in FIG. 6A.

As discussed with respect to FIG. 6A, the system 1400 of FIG. 14 includes the sequencing machine 1404, such as the sequencing machine discussed with respect to FIG. 1. The sequencing machine 1404 includes a flow cell 1405, such as the flow cell discussed with respect to FIGS. 1-3. The flow cell 1405 includes a plurality of tiles 1406, and each tile 1406 includes a plurality of clusters 1407 (example clusters of a single tile are illustrated in FIG. 6A), e.g., as discussed with respect to FIGS. 2 and 3. As discussed with respect to FIGS. 4-6, sensor data 1412 comprising raw images from the tiles 1406 are output by the sequencing machine 1404.

In an embodiment, the system 1400 comprises two or more base callers, such as a first base caller 1414 and a second base caller 1416. Although two base callers are illustrated in the figure, in an example, more than two base callers may be present in the system 1400, such as three, four, or a higher number of base callers.

In an example, the base callers 1414 and 1416 are local to the sequencing machine 1404. Thus, the base callers 1414 and 1416 and the sequencing machine 1404 are proximally located (e.g., within a same housing, or within two proximally located housing), and the base callers 1414 and 1416 receive the sensor data 1412 directly from the sequencing machine 1404.

In another example, the base callers 1414 and 1416 are located remotely relative to the sequencing machine 1404, which is an example of the so-called cloud-based base callers. Thus, the base callers 1414 and 1416 receive the sensor data 1412 from the sequencing machine 1404 via a computer network, such as the Internet.

Each base caller 1414 and 1416 of FIG. 14 outputs corresponding base call classification information. For example, the first base caller 1414 outputs the first base call classification information 1434, and the second base caller 1416 outputs the second base call classification information 1436. A base calling combining module 1428 generates final base calls 1440, based on one or both the first base call classification information 1434 and the second base call classification information 1436.

In an example, the first base caller 1414 is a neural network based base-caller. For example, the first base caller 1414 is a non-linear system employing one or more neural network models for base calling, as discussed herein previously (e.g., see FIGS. 6-13B).

In an example, the second base caller 1416 is a non-neural network based base-caller. For example, the second base caller 1416 is at least in part a linear system used for base calling. For example, the second base caller 1416 does not employ a neural network for base calling (or uses a smaller neural network model for base calling, compared to a larger neural network model used by the first base caller 1414), as discussed herein previously (e.g., see FIG. 6 and subsequent discussion).

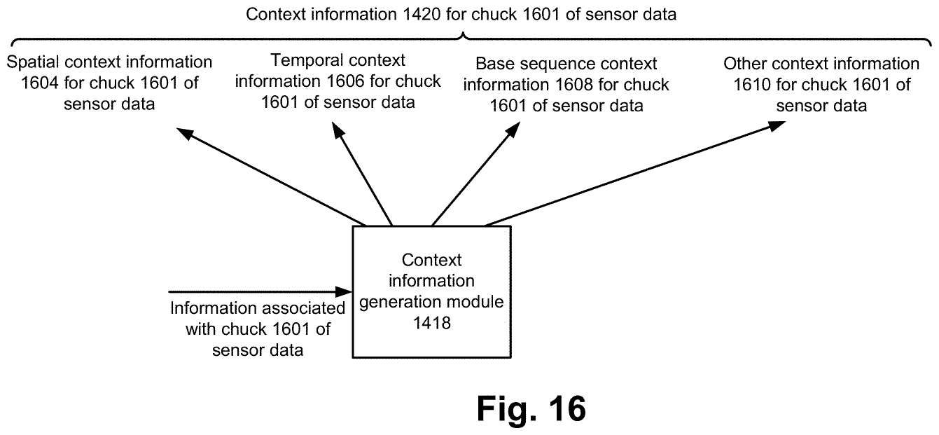

In an embodiment, the system 1400 comprises a context information generation module 1418. The context information generation module 1418 generates context information 1420. In an embodiment, the base calling combining module 1428 operates based on the context information 1420. For example, based on the context information 1420, the base calling combining module 1428 uses one or both the base call classification information 1434 and the base call classification information 1436, to generate the final base call. Context information will be discussed herein later, e.g., with respect to FIG. 16.

In an embodiment, the system 1400 also includes a switching module 1422. Note that in FIG. 14, the switching module 1422, the context information generation module 1418, and the base calling combining module 1428 are illustrated as three separate components of the system 1400. However, in an example, one or more of these modules can be combined to form a combined module.

In an embodiment, the system 1400 also includes the switching module 1422 that selectively switches on or off the base callers 1414 and 1416. For example, depending on the context information 1420, if only one of the base callers 1414 and 1416 is supposed to analyze a specific set of sensor data 1412, then for that set of sensor data only the selected base caller is enabled and the other base caller is disabled, as will be discussed herein later in further detail.

Enabling or switching on a base caller for a set of sensor data implies that the base caller is to operate or execute on the specific set of sensor data. Thus, enabling or switching on a base caller does not necessarily imply turning the base caller on—it merely implies that the base caller is executed on a specific corresponding set of sensor data. Disabling or switching off a base caller for a set of sensor data implies that the base caller is to refrain from operating or executing on the specific set of sensor data. Note that, for example, while a base caller is disabled for a first set of sensor data, the base caller may be enabled for a second set of sensor data. In an example, the first base caller 1414 can be selectively enabled or disabled using the enable signal 1424, and the second base caller 1416 can be selectively enabled or disabled using the enable signal 1426. Thus, the enable signals 1424 and 1426 are signals to selectively enable (or disable) the corresponding base callers 1414 or 1416, respectively.

A “set of sensor data,” as discussed herein, refers to a section of sensor data 1412, or a dataset of sensor data 1412. For example, a set of sensor data may be sensor data from one or more specific clusters 1407, or one or more specific tiles 1406, of the flow cell 1405. A set of sensor data may be sensor data from one or more specific base sensing cycles. Thus, a set of sensor data may be associated with a specific spatial aspect of the flow cell 1405 (e.g., from one or more specific clusters 1407 of the flow cell 1405) and/or a specific temporal aspect of base calling cycles (e.g., from one or more specific base calling cycles).

Merely as an example, for a first set of sensor data 1412, the base calling combining module 1428 may rely only on the base call classification information 1434 from the first base caller 1414, to generate the final base call 1440 for the first set of sensor data 1412. The base calling combining module 1428 decides to rely solely on the base call classification information 1434 (and not on the base call classification information 1436), e.g., based on the context information 1420 associated with the first set of sensor data 1412. In an example, when processing the first set of sensor data, the switching module 1422 only enables the first base caller 1414 using the enable signal 1424 (e.g., the first base caller 1414 executes on the first set of data), and disables the second base caller 1416 using the enable signal 1426 (e.g., the second base caller 1416 does not execute on the first set of data), and the first base call classification information 1434 from the first base caller 1414 is used to generate the final base call 1440. However, in another example, although the first base call classification information 1434 from the first base caller 1414 is used to generate the final base call 1440 for the first set of data, the switching module 1422 enables the first base caller 1414, and optionally also enables the second base caller 1416, e.g., for reasons discussed herein later. In such an example, both the base call classification information 1434 and 1436 are available for the first set of data, and the final base call 1440 is based solely on the first base call classification information 1434.

Merely as another example, for a second set of sensor data 1412, the base calling combining module 1428 may rely only on the base call classification information 1436 from the second base caller 1416, to generate the final base call 1440 for the second set of sensor data 1412. The base calling combining module 1428 decides to rely solely on the base call classification information 1436 (and not on the base call classification information 1434), e.g., based on the context information 1420 associated with the second set of sensor data 1412. In an example, when processing the second set of sensor data, the switching module 1422 only enables the second base caller 1416 using the enable signal 1426, and disables the first base caller 1414 using the enable signal 1424, e.g., and the second base call classification information 1436 from the second base caller 1416 is used to generate the final base call 1440. However, in another example, although the second base call classification information 1436 from the second base caller 1416 is used to generate the final base call 1440 for the second set of data, the switching module 1422 enables the second base caller 1416, and optionally also enables the first base caller 1414, e.g., for reasons discussed herein later. In such an example, both the base call classification information 1434 and 1436 are available, and the final base call 1440 is based solely on the base call classification information 1436.

Merely as yet another example, for a third set of sensor data 1412, the base calling combining module 1428 may rely on both the base call classification information 1434 and 1436 from the base callers 1414 and 1416, respectively, to generate the final base call 1440 for the third set of sensor data 1412. The base calling combining module 1428 decides to rely on both the base call classification information 1434 and 1436, e.g., based on the context information 1420 associated with the third set of sensor data 1412. Thus, when processing the third set of sensor data, the switching module 1422 enables both base callers 1414 and 1416 using the enable signals 1424 and 1426, respectively.

Thus, for a given set of sensor data, the base calling combining module 1428 decides to rely on a specific one, or both, the base call classification information 1434 and 1436, based on the context information 1420 associated with the corresponding set of sensor data. Similarly, the switching module 1422 decides to enable a specific one, or both, the base callers 1414 and 1416, based on the context information 1420 associated with the corresponding set of sensor data.

### Example Operations of the First Base Caller 1414 and Second Base Caller 1416

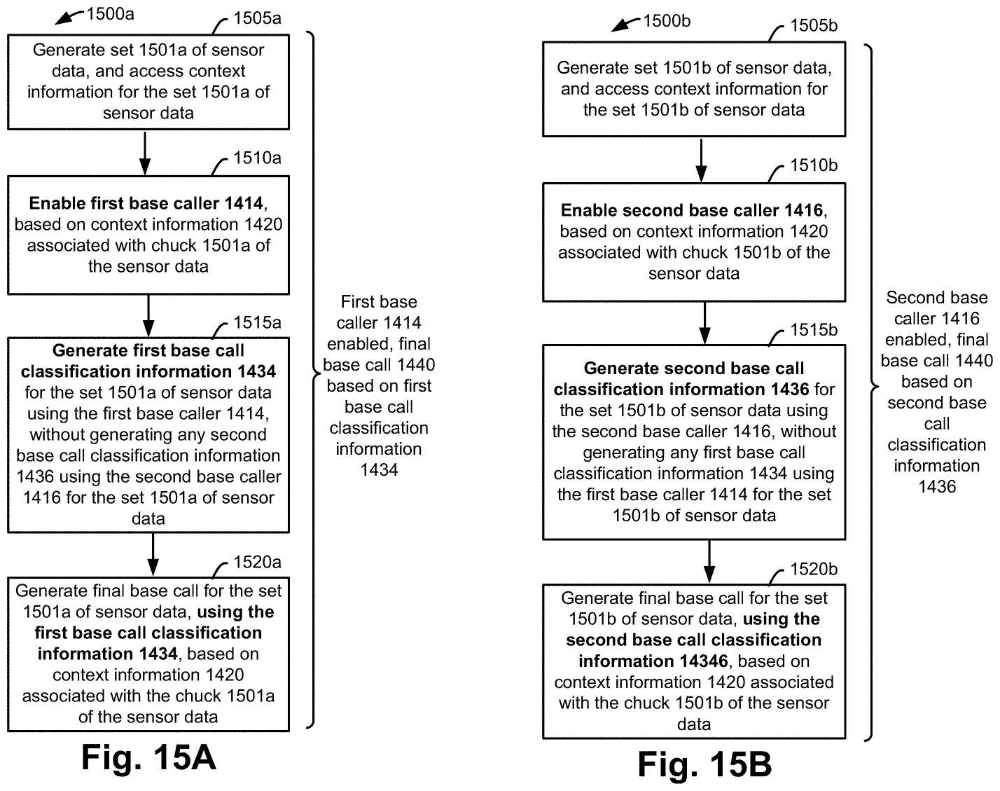

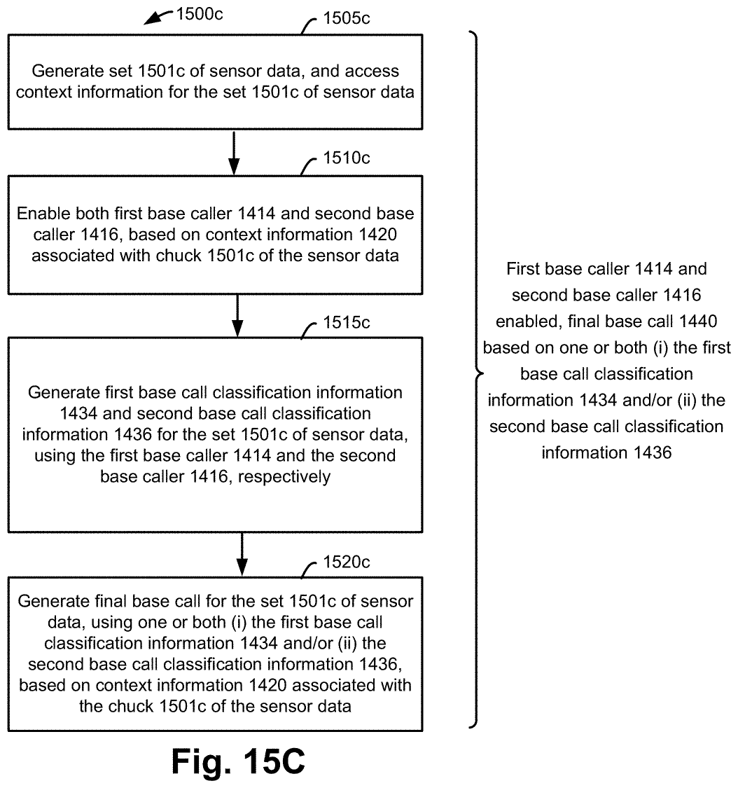

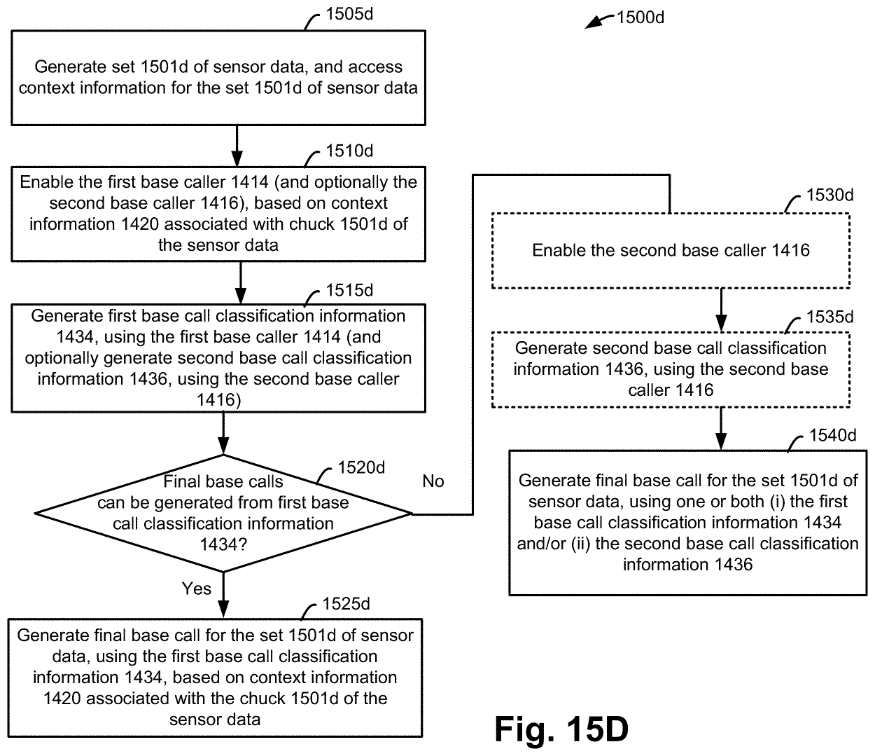

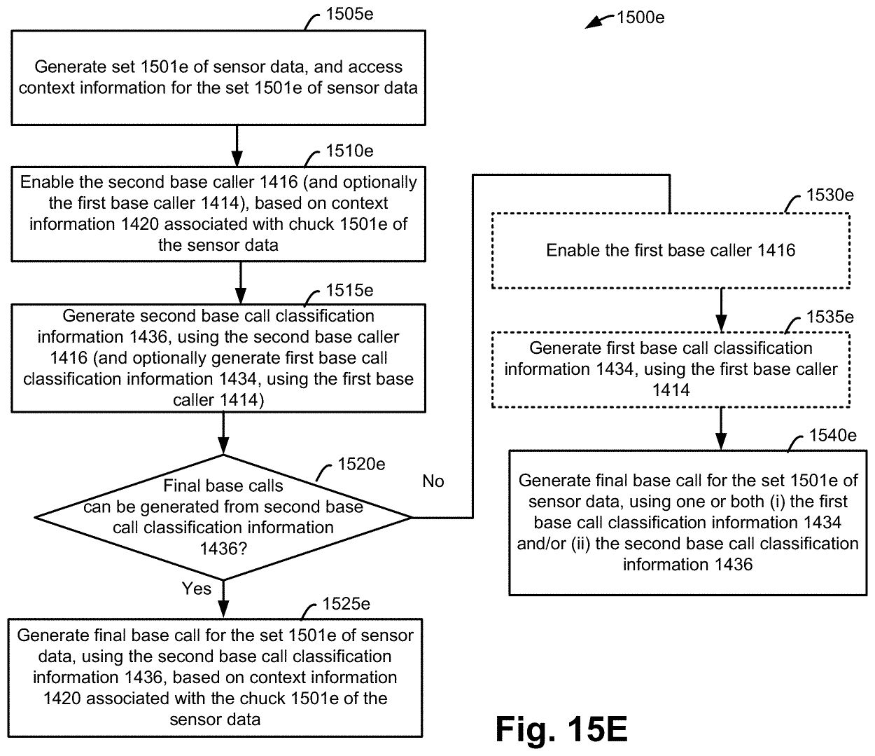

FIGS. 15A, 15B, 15C, 15D, and 15E illustrate corresponding flow charts that depict various operations of the base calling system 1400 of FIG. 14 for corresponding sets of sensor data. For example, FIGS. 15A-15E illustrate various permutations and combinations in which the system 1400 may operate.

### First Base Caller 1414 Enabled, Final Base Call 1440 Based on First Base Call Classification Information 1434

FIG. 15A illustrates operations of the system 1400, in which the first base caller 1414 is enabled and generates base call classification information for a set 1501a of sensor data (e.g., while the second base caller 1416 is non-operational on the set 1501a of sensor data), and the final base call 1440 is based on the first base call classification information 1434 for the set 1501a of sensor data.

Thus, in FIG. 15A, operations of the system 1400 are illustrated for the set 1501a of sensor data generated by the flow cell 1405. At 1505a, the flow cell 1405 generates a set 1501a of sensor data. As discussed, the set 1501a of sensor data can be generated in a specific location of the flow cell, such as by specific clusters of specific tiles, or by specific tiles, and for specific base sequence cycles (i.e., a set is associated with a specific spatial location(s) of the flow cell 1405 and a specific temporal base sequence cycle(s)). Also at 1505a, context information associated with the set 1501a of sensor data is accessed (e.g., by the switching module 1422 and/or the base calling combining module 1428). As discussed, the context information may be generated by the context information generation module 1418.

In the example of FIG. 15A, the switching module 1422 decides that the first base caller 1414 (and not the second base caller 1416) is to process the set 1501a of sensor data. Accordingly, at 1510a, the switching module 1422 enables the first base caller 1414, e.g., by turning on the enable signal 1424. The second base caller 1416 may remain disabled, i.e., the second base caller 1416 does not operate on the set 1501a of sensor data.

At 1515a, the first base caller 1414 generates the first base call classification information 1434 for the set 1501a of sensor data, while the second base caller 1416 refrains from generating any second base call classification information 1436 for the set 1501a of sensor data.

At 1520a, the base calling combining module 1428 generates a final base call for the set 1501a of sensor data, using the first base call classification information 1434, based on context information 1420 associated with the set 1501a of the sensor data.

### Second Base Caller 1416 Enabled, Final Base Call 1440 Based on Second Base Call Classification Information 1436

FIG. 15B illustrates operations of the system 1400, in which the second base caller 1416 is enabled and generates base call classification information for a set 1501b of sensor data (e.g., while the first base caller 1414 is non-operational on the set 1501b of sensor data), and the final base call 1440 is based on the second base call classification information 1436 for the set 1501b of sensor data.

At 1505b, the flow cell 1405 generates a set 1501b of sensor data. As discussed, the set 1501b of sensor data can be generated in a specific location of the flow cell, such as by specific clusters of specific tiles, or by specific tiles, and for specific base sequence cycles (i.e., a set is associated with a specific spatial location(s) of the flow cell 1405 and a specific temporal base sequence cycle(s)). Also at 1505b, context information associated with the set 1501b of sensor data is accessed (e.g., by the switching module 1422 and/or the base calling combining module 1428). As discussed, the context information may be generated by the context information generation module 1418.

In the example of FIG. 15B, the switching module 1422 decides that the second base caller 1416 (and not the first base caller 1414) is to process the set 1501b of sensor data. Accordingly, at 1510b, the switching module 1422 enables the second base caller 1416, e.g., by using the enable signal 1426. The first base caller 1414 may remain disabled, i.e., the first base caller 1414 does not operate on the set 1501b of sensor data.

At 1515b, the second base caller 1416 generates the second base call classification information 1436 for the set 1501b of sensor data, while the first base caller 1414 refrains from generating any first base call classification information 1434 for the set 1501b of sensor data.

At 1520b, the base calling combining module 1428 generates a final base call for the set 1501b of sensor data, using the second base call classification information 1436, based on context information 1420 associated with the set 1501b of the sensor data.

First Base Caller 1414 and Second Base Caller 1416 Enabled, Final Base Call 1440 Based on One or Both (i) the First Base Call Classification Information 1434 and/or (ii) the Second Base Call Classification Information 1436

FIG. 15C illustrates operations of the system 1400, in which both the first base caller 1414 and the second base caller 1416 are enabled (i.e., both base callers operate on the corresponding set 1501c of sensor data) and generate corresponding base call classification information for the set 1501c of sensor data, and the final base call 1440 is based on one or both of (i) the first base call classification information 1434 and/or (ii) the second base call classification information 1436.

At 1505c, the flow cell 1405 generates a set 1501c of sensor data. As discussed, the set 1501c of sensor data can be generated in a specific location(s) of the flow cell, such as by specific clusters of specific tiles, or by specific tiles, and for a specific base sequence cycle(s) (i.e., a set is associated with a specific spatial location(s) of the flow cell 1405 in FIG. 14 and a specific temporal base sequence cycle(s)). Also at 1505c, context information associated with the set 1501c of sensor data is accessed (e.g., by the switching module 1422 and/or the base calling combining module 1428). As will be discussed in further detail herein, the context information may be generated by the context information generation module 1418.

In the example of FIG. 15C, the switching module 1422 decides that both the first base caller 1414 and the second base caller 1416 are to process the set 1501c of sensor data. Accordingly, at 1510c, the switching module 1422 (FIG. 14) enables both the first base caller 1414 and the second base caller 1416, e.g., using the enable signals 1424 and 1426 (FIG. 14). For example, both the first base caller 1414 and the second base caller 1416 are to process the entire set 1501c of sensor data. In another example, the first base caller 1414 is to process a first subset of the set 1501c of sensor data, and the second base caller 1416 is to process a second subset of the set 1501c of sensor data.

At 1515c, the first base caller 1414 generates the first base call classification information 1434 for the set 1501c of sensor data, and the second base caller 1416 generates the second base call classification information 1436 for the set 1501c of sensor data.

At 1520c, the base calling combining module 1428 generates a final base call for the set 1501b of sensor data, using the first base call classification information 1434 and/or the second base call classification information 1436, based on context information 1420 associated with the set 1501c of the sensor data.

### Enable and Use the Second Base Call Classification Information 1436, in Case the Final Base Calls Cannot be Generated Solely Using the First Base Call Classification Information 1434

FIG. 15D illustrates operations of the system 1400, in which the second base call classification information 1436 is used for the final base calls 1440, in case the final base calls cannot be generated solely using the first base call classification information 1434.

At 1505d, the flow cell 1405 generates a set 1501d of sensor data. As discussed, the set 1501d of sensor data can be generated in a specific location of the flow cell, such as by specific clusters of specific tiles, or by specific tiles, and for specific base sequence cycles (i.e., a set is associated with a specific spatial location(s) of the flow cell 1405 and a specific temporal base sequence cycle(s)). Also at 1505d, context information associated with the set 1501d of sensor data is accessed (e.g., by the switching module 1422 and/or the base calling combining module 1428). As discussed, the context information may be generated by the context information generation module 1418.

In the example of FIG. 15D, the switching module 1422 decides that the first base caller 1414 is to process the set 1501d of sensor data. Optionally, the switching module 1422 may also decide that the second base caller 1416 may also process the set 1501d of sensor data. Thus, at 1510d, the first base caller 1414 is enabled, and optionally the second base caller 1416 is also enabled.

At 1515d, the first base caller 1414 generates the first base call classification information 1434 for the set 1501d of sensor data. In the optional operation at 1510d in which the second base caller 1416 is enabled, the second base caller 1416 optionally generates the second base call classification information 1436 for the set 1501d of sensor data.

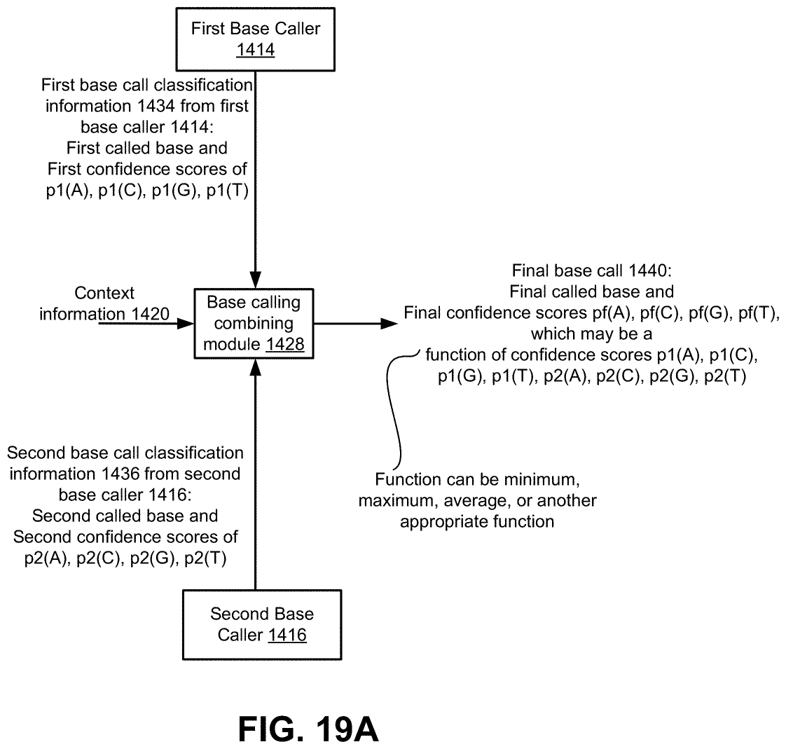

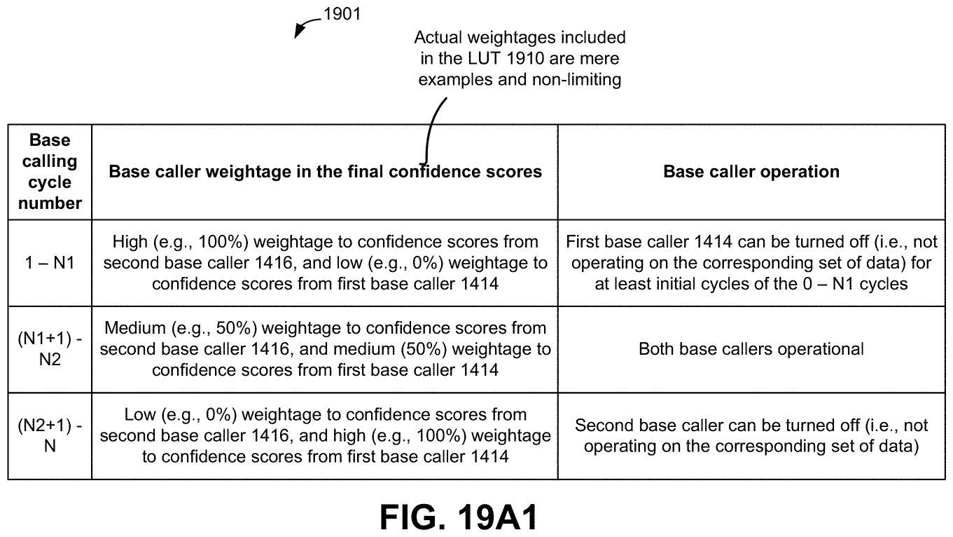

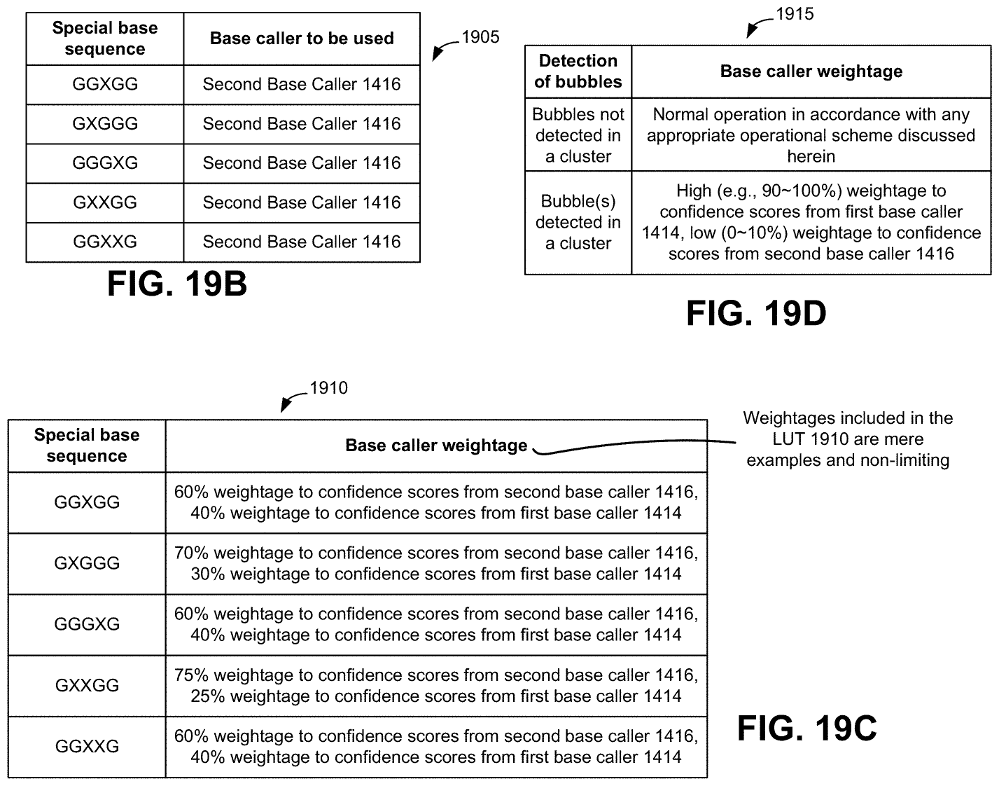

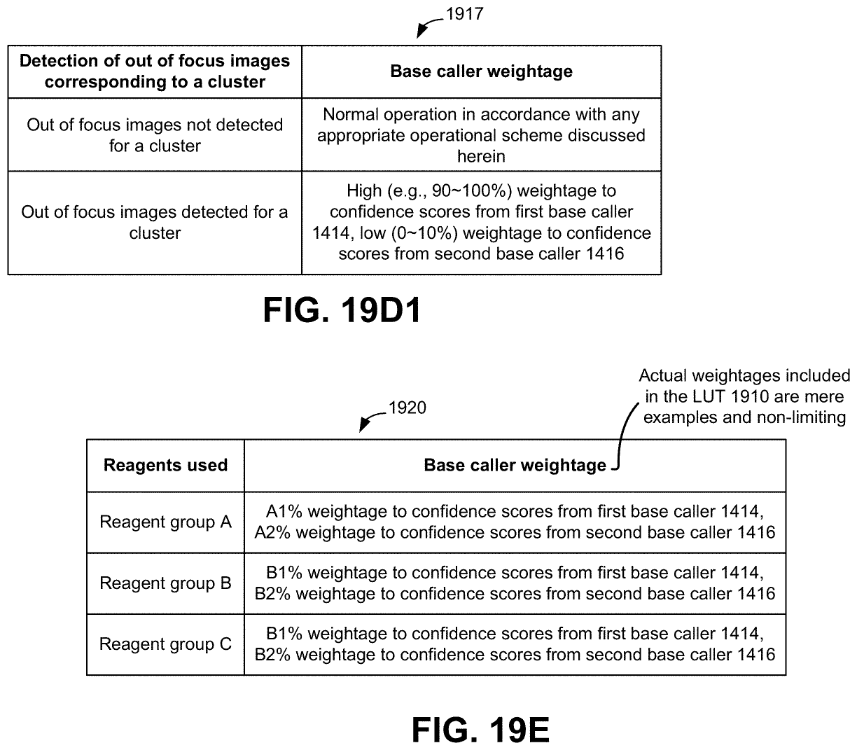

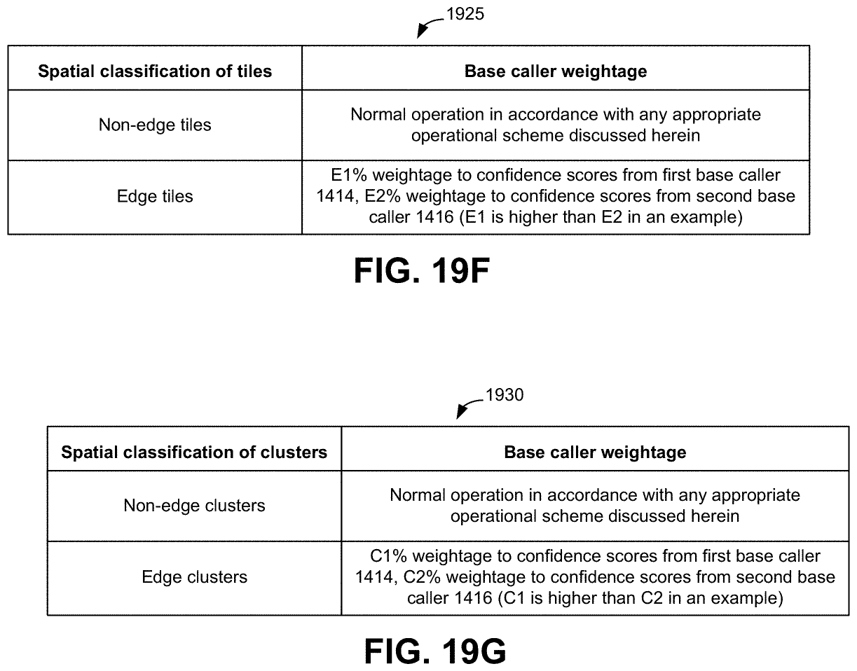

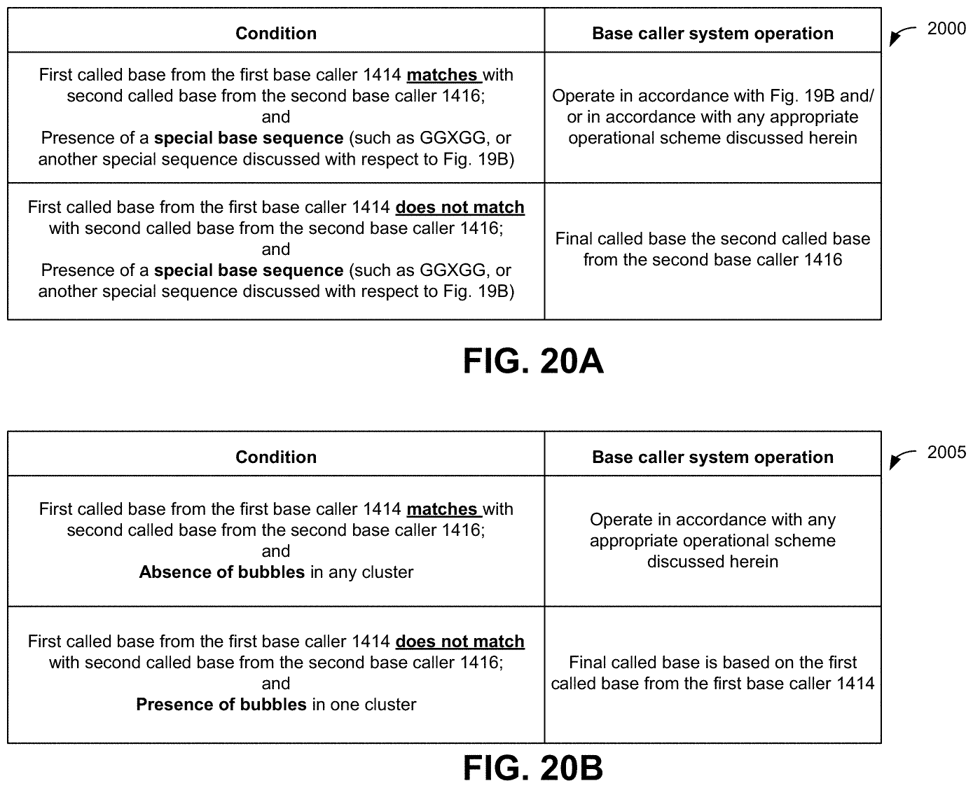

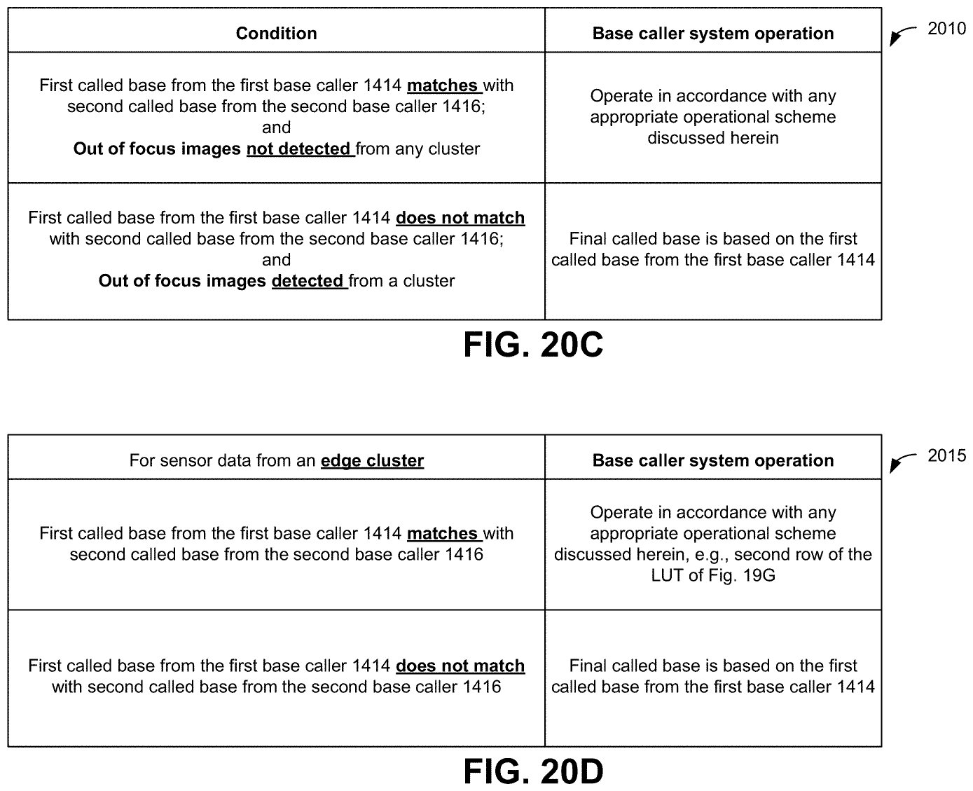

At 1520d, a determination is made (e.g., by the switching module 1422 and/or the base calling combining module 1428) as to whether the final base calls can be generated from the first base call classification information 1434 (e.g., without using the second base call classification information 1436). For example, it may be determined that a probability of error in the final base calls 1440 may be relatively high, e.g., if the final base calls 1440 are based solely on the first base call classification information 1434. Numerous examples of such a determination will be discussed herein later in turn. Merely as an example, if the first base call classification information 1434 indicates a homopolymer (e.g., GGGGG) or near-homopolymer (e.g., GGTGG) sequence, then the first base call classification information 1434 may be insufficient or inadequate for generating the final base calls (e.g., the second base call classification information 1436 has to be relied upon for generating the final base calls), e.g., as discussed herein later with respect to FIGS. 19B and 19C.

If “Yes” at 1520d (i.e., the final base calls can be generated from the first base call classification information 1434, without using the second base call classification information 1436), then the method 1500d proceeds to 1525d, where the final base call for the set 1501d of sensor data is generated using the first base call classification information 1434.

If “No” at 1520d (i.e., the final base calls cannot be generated solely from the first base call classification information 1434, e.g., without using the second base call classification information 1436), then the method 1500d proceeds to 1530d, where the second base caller 1416 is enabled, and then at 1535d where the second base call classification information 1436 is generated, using the second base caller 1416. Note that operations at blocks 1530d and 1535d are optional, and hence, illustrated using dotted lines. For example, if the second base caller 1416 was optionally enabled at 1510d, then the operation 1530d can be skipped. Similarly, if the second base call classification information 1436 was optionally generated using the second base caller 1416 at 1515d, then the operation 1535d can be skipped.

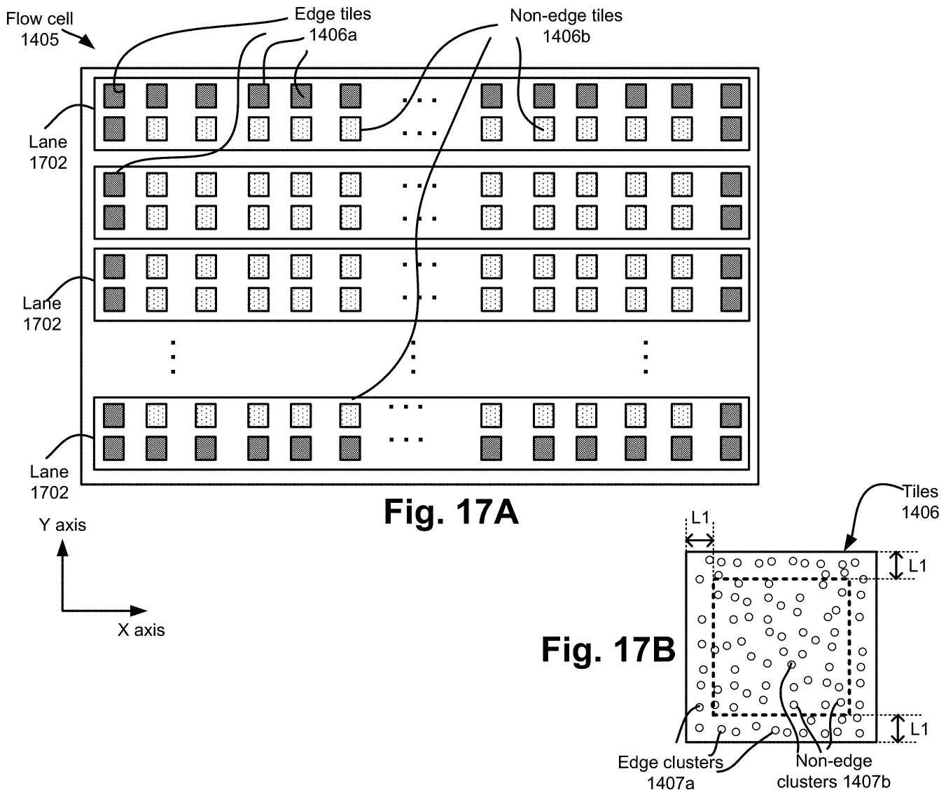

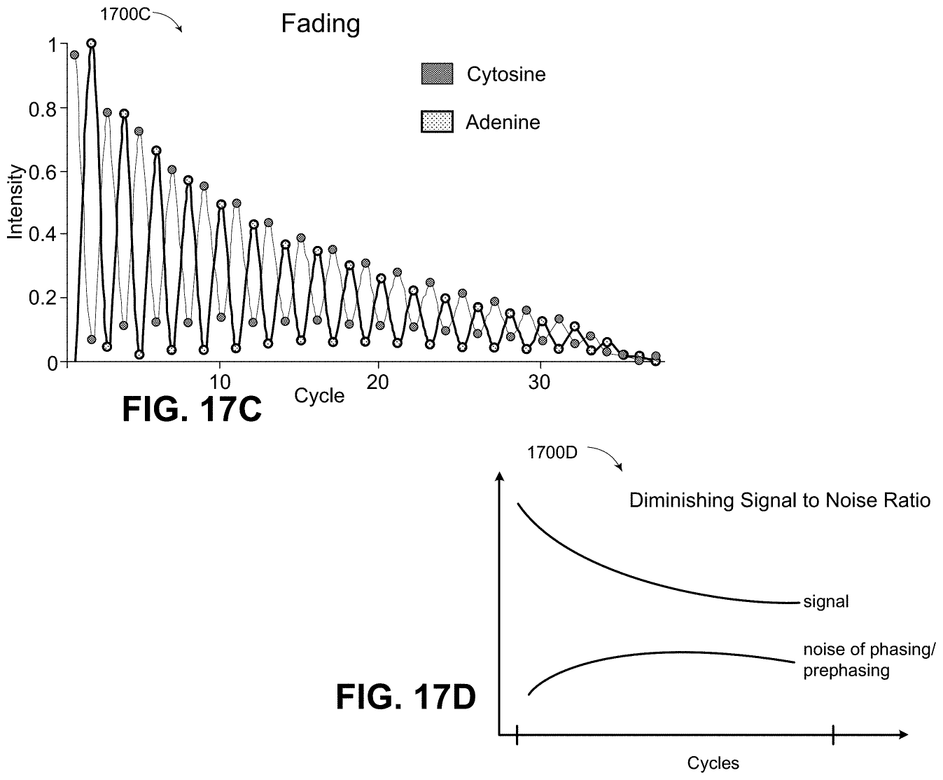

Assume a scenario where the second base caller 1416 was not enabled at 1510d, and the second base caller 1416 is enabled at 1530d. Thus, at 1530d, the second base caller 1416 starts processing the set 1510d of sensor data. It may be noted that the for a given base calling cycle, the second base caller 1416 cannot immediately start processing the corresponding sensor data and generate base calls. This is because, due to phasing discussed herein later (e.g., see FIGS. 17C, 17D), the second base caller 1416 has to process sensor data of one or more previous base calling cycles to satisfactorily call the base of a current cycle. For example, assume base call cycles 1 to 1000 are to be performed, and the set 1501d of sensor data comprises images from base calling cycle 100 onwards. Also assume, at 1530d, the second base caller 1416 is enabled to process sensor data for base calling cycle 100 and one or more subsequent base calling cycles. As discussed, the second base caller 1416 has to process sensor data from one or more previous cycles to satisfactorily call bases for the cycle 100 and subsequent cycles. Processing sensor data from a few previous cycles enables the second base caller 1416 to estimate the effects of phasing at cycle 100, which increases a quality of base calling at cycle 100. Merely as an example, five, ten, twenty, or another appropriate number of previous cycles are to be processed by the second base caller 1416, for the second base caller 1416 to satisfactorily call bases for the cycle 100.

Assume, in a first example, that the second base caller 1416 has process sensor data from N1 number of previous cycles to satisfactorily call bases for the cycle 100. Assume, in a second example, that the second base caller 1416 has process sensor data from N2 number of previous cycles to satisfactorily call bases for cycle 1000. Now, as will be discussed with respect to FIGS. 17C, 17D, the effects of phasing and pre-phasing are more pronounced as the base calling cycles progress. Thus, phasing and pre-phasing are more pronounced in cycle 1000 than the cycle 100. Thus, to satisfactorily call bases for cycle 1000, the second base caller 1416 has to process a higher number of previous cycles than a number of previous cycles to be processed to satisfactorily call bases for cycle 100. Thus, N2 is higher than N1.

Referring again to FIG. 15D, subsequent to 1535d, at 1540d, the final base call for the set 1501d of sensor data is generated, using one or both (i) the first base call classification information 1434 and/or (ii) the second base call classification information 1436.

### Enable and Use the First Base Call Classification Information 1434, in Case the Final Base Calls Cannot be Generated Solely Using the Second Base Call Classification Information 1436

FIG. 15E illustrates operations of the system 1400, in which the first base call classification information 1434 is used for the final base calls 1440, in case the final base calls cannot be generated solely using the second base call classification information 1436.

At 1505e, the flow cell 1405 generates a set 1501e of sensor data. As discussed, the set 1501e of sensor data can be generated in specific location of the flow cell, such as by specific clusters of specific tiles, or by specific tiles, and for specific base sequence cycles (i.e., a set is associated with specific spatial locations of the flow cell 1405 and specific temporal base sequence cycles). Also at 1505e, context information associated with the set 1501e of sensor data is accessed (e.g., by the switching module 1422 and/or the base calling combining module 1428). As discussed, the context information may be generated by the context information generation module 1418.

In the example of FIG. 15E, the switching module 1422 decides that the second base caller 1416 is to process the set 1501e of sensor data, e.g., based on the associated context information. Optionally, the switching module 1422 may also decide that the first base caller 1414 may also process the set 1501e of sensor data. Thus, at 1510e, the second base caller 1416 is enabled, and optionally the first base caller 1414 is also enabled.

At 1515e, the second base caller 1416 generates the second base call classification information 1436 for the set 1501e of sensor data. In the option in which the first base caller 1414 is also enabled, the first base caller 1414 generates the first base call classification information 1434 for the set 1501e of sensor data.

At 1520e, a determination is made (e.g., by the switching module 1422 and/or the base calling combining module 1428) as to whether the final base calls can be generated solely from the second base call classification information 1436 (e.g., without using the first base call classification information 1434). For example, it may be determined (e.g., based on the context information) that a probability of error in the final base calls 1440 may be relatively high, e.g., if the final base calls 1440 are based solely on the second base call classification information 1436. Numerous examples of such a determination will be discussed herein later in turn. Merely as an example, if the context information indicates detection of bubbles in a cluster, then the final base calls cannot be generated from the second base call classification information 1436 (e.g., without using the first base call classification information 1434), e.g., as discussed herein later with respect to FIG. 19D.

If “Yes” at 1520e (i.e., the final base calls can be generated from the second base call classification information 1436, without using the first base call classification information 1434), then the method 1500e proceeds to 1525e, where the final base call for the set 1501e of sensor data is generated using the second base call classification information 1436.

If “No” at 1520e (i.e., the final base calls cannot be generated from the second base call classification information 1436, e.g., without using the first base call classification information 1434), then the method 1500e proceeds to 1530e, where the first base caller 1414 is enabled, and then to 1535e where the first base call classification information 1434 is generated using the first base caller 1414. Note that operations at blocks 1530e and 1535e are optional, and hence, illustrated using dotted lines. For example, if the first base caller 1414 was optionally enabled at 1510e, then the operation 1530e can be skipped. Similarly, if the first base call classification information 1434 was optionally generated using the first base caller 1414 at 1515e, then the operation 1535e can be skipped.

Assume a scenario where the first base caller 1414 was not enabled at 1510e, and the first base caller 1414 is enabled at 1530e. Thus, at 1530e, the first base caller 1416 starts processing the set 1510e of sensor data. It may be noted that for a given base calling cycle, the first base caller 1414 cannot immediately start processing the corresponding sensor data and generate base calls. For example, assume the first base caller 1414 is to operate on a corresponding set of data from base calling cycle Na. To satisfactorily generate base calls from cycle Na, the first base caller 1414 also has to operate on sensor data from at least a few cycles before the cycle Na, e.g., because, as discussed with respect to FIGS. 7 and 10, a base call for a current cycle is also based on data from one or more past cycles and one or more future cycles. Accordingly, to generate the first base call classification information 1434 from cycle Na, the first base caller 1414 has to also process sensor data from a few prior cycles (such as 2 cycles in the example of FIG. 7, and five cycles in the example of FIG. 10).

Subsequently, at 1540e, the final base call for the set 1501e of sensor data is generated using one or both of (i) the first base call classification information 1434 and/or (ii) the second base call classification information 1436.

### Context Information

FIG. 16 illustrates the context information generation module 1418 of the base calling system 1400 of FIG. 14 generating context information 1420 for an example set 1601 of sensor data. For example, the context information generation module 1418 receives information about the set 1601 of sensor data, and generates various types of context information for the set 1601 of sensor data, which, in combination, are referred to as the context information for the set 1601 of sensor data. For example, the context information generation module 1418 generates spatial context information 1604, temporal context information 1606, base sequence context information 1608, and other context information 1610 for the set 1601 of sensor data.

### Spatial Context Information 1604

As the name suggests, the spatial context information 1604 refers to context information associated with the spatial location of the tiles and clusters from which the set 1601 of sensor data is generated. FIGS. 17A and 17B below discuss examples of spatial context information 1604.

FIG. 17A illustrates the flow cell 1405 of the system 1400 of FIG. 14, where the flow cell 1405 comprises tiles 1406 that are categorized based on the spatial location of the tiles. For example, as discussed with respect to FIG. 2, the flow cell 1405 of FIG. 17A comprises a plurality of lanes 1702, with a corresponding plurality of tiles 1406 within each lane. FIG. 17A illustrates a top view of the flow cell 1405.

Individual tiles are categorized based on the location of the tiles. For example, tiles that are adjacent to any edge of the flow cell 1405 are labeled as edge tiles 1406a (illustrated using grey boxes), and the remaining tiles are labeled as non-edge tiles 1406b (illustrated using dotted boxes).

For example, tiles that are on vertical (e.g., along the Y-axis) and/or horizontal (e.g., along the X-axis) edges of the flow cell 1404 are categorized as edge tiles 1406a, as illustrated in FIG. 14. Thus, an edge tile 1406a is adjacent (e.g., immediate adjacent) to a corresponding edge of the flow cell 1404, and a non-edge tile is not adjacent to any edge of the flow cell 1404.

Base calling cycles are performed for clusters in individual tiles of the flow cell 1404. In an example, parameters relevant for base calling operations for a tile can be based on a relative location of the tile. For example, the excitation light 101 discussed with respect to FIG. 1 is directed towards the tiles of the flow cell, and different tiles can receive different amount of the excitation light 101, e.g., based on the location of an individual tile and/or the location of one or more light sources emitting the excitation light 101. For example, if the light source(s) emitting the excitation light 101 is vertically above the flow cell, non-edge tiles 1406b may receive a different amount of light than the edge tiles 1406a. In another example, peripheral or external light (e.g., ambient light from outside the biosensor) around the flow cell 1405 can influence the amount and/or characteristics of the excitation light 101 received by individual tiles of the flow cell 1405. Merely as an example, the edge tiles 1406a may receive the excitation light 101, along with some amount of peripheral light from outside the flow cell 1405, whereas the non-edge tiles 1406b may mainly receive the excitation light 101. In yet another example, individual sensors (or pixels or photodiodes) included in the flow cell 1405 (e.g., sensors 106, 108, 110, 112, and 114 illustrated in FIG. 1) can sense light based on the locations of the corresponding sensors, which are based on the location of the corresponding tiles. For example, sensing operations performed by one or more sensors associated with the edge tiles 1406a can be influenced by peripheral light (along with the excitation light 101) relatively more than an effect of the peripheral light on sensing operations of one or more other sensors associated with the non-edge tiles 1406b. In yet another example, the flow of the reactant (e.g., which includes any substance that may be used to obtain a desired reaction during the base calling, such as reagents, enzymes, samples, other biomolecules, and buffer solutions) to various tiles can also be influenced by tile location. For example, tiles near a source of the reactants can receive a higher amount of reactants than tiles that are further from the source.

In an example, the spatial context information 1604 associated with the set 1601 of sensor data (see FIG. 16) includes information regarding whether the set 1601 of sensor data was generated in an edge tile 1406a or a non-edge tile 1406b. As discussed above, parameters associated with base calling may be slightly different for different categories of tiles. Accordingly, in an embodiment, spatial context information 1604 indicating whether the set 1601 of sensor data is generated from an edge tile or a non-edge tile can influence the selection of a base caller for processing the set 1601 of sensor data. Merely as an example and based on the implementation details, the first base caller 1414 may be more suitable for processing sensor data from one of edge or non-edge tiles, whereas the second base caller 1416 may be more suitable for processing sensor data from another of edge or non-edge tiles.

FIG. 17B illustrates a tile 1406 of the flow cell 1405 of the system 1400 of FIG. 14, where the tile 1406 comprises clusters 1407 that are categorized based on the spatial location of the clusters. p In an example, based on the sensor data (which may be image data, for example) received from a tile, locations of various clusters within the tile can be estimated. For example, the location of an individual cluster can be identified using the (x,y) coordinates of the cluster. Thus, each cluster 1407 has corresponding (x,y) coordinates identifying the location of the cluster relative to the tile. In FIG. 17B, the clusters 1407 of the example tile 1406 are categorized as either edge clusters 1407a or non-edge clusters 1407b. For example, clusters 1407 that are within a threshold distance L1 from an edge of the tile are labeled as edge clusters 1407a, and clusters 1407 that are outside the threshold distance L1 from an edge of the tile are labeled as non-edge clusters 1407b. Thus, edge clusters 1407a are located near a periphery of the tile 1406, whereas non-edge clusters 1407a are located near a central section of the tile 1406. As discussed, using the (x,y) coordinates of a cluster, a distance of the cluster relative to an edge of the tile can be determined (e.g., by the context information generation module 1418), based on which the context information generation module 1418 classifies the cluster to be either an edge cluster 1407a or a non-edge cluster 1407b. As a simple example, illustrated in FIG. 17B is an imaginary dotted rectangle that is within a periphery of the tile 1406 and at distance L1 from the periphery of the tile 1406. Clusters within the dotted rectangle are classified as non-edge clusters 1407b, whereas clusters between the periphery of the dotted rectangle and the periphery of the tile 1406 are classified as edge clusters 1407a.

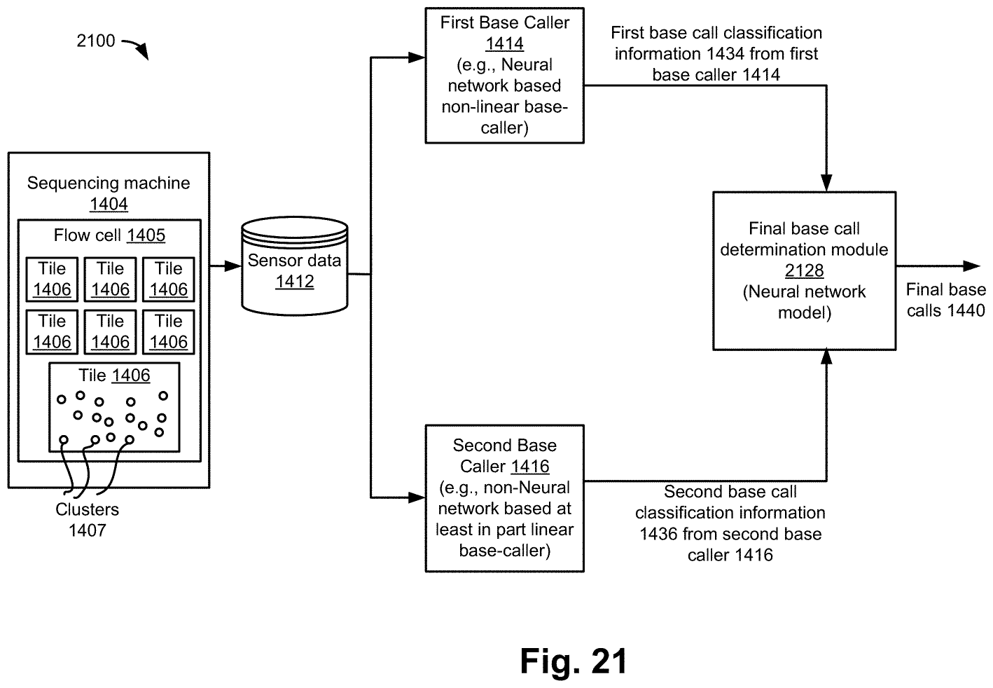

As discussed with respect to FIG. 1, the flow cell 1405 may include a lens (such as the filter layer 124 including an array of microlenses or other optical components) for capturing images of various clusters. In an example, there may be slight difference in focusing for various clusters when capturing the images, e.g., as the image sensor or camera is moved around the flow cell. For example, edge clusters 1407a may be slightly out of focus, relative to the non-edge clusters 1407b, when images of the clusters are captured. Out of focus events may also occur due to heating or mechanical vibration cause by the movement of the lens. Thus, depending on the implementation, one of the first or second base callers 1414 or 1416 may be better suited to process sensor data from edge clusters 1407a, while another of the first or second base callers 1414 or 1416 may be better suited to process sensor data from non-edge clusters 1407b, as will be discussed herein later in further detail (see FIGS. 19G, 20D). In an example, spatial context information 1604 associated with the set 1601 of sensor data (see FIG. 16) includes information regarding whether the set 1601 of sensor data was generated from one or more edge clusters 1407a or one or more non-edge clusters 1407b, based on which the set 1601 of sensor data can be processed by a specific one, or both, the first or second base callers 1414 or 1416.

Thus, summarizing the above discussion, spatial context information 1604 associated with the set 1601 of sensor data includes (i) information regarding whether the set 1601 of sensor data was generated from an edge tile 1406a or a non-edge tile 1406b, and/or (ii) information regarding whether the set 1601 of sensor data was generated from one or more edge clusters 1407a or one or more non-edge clusters 1407b. Other appropriate spatial context information may also be envisioned, based on the teachings of this disclosure.

### Temporal Context Information 1606

Referring again to FIG. 16, the context information generation module 1418 also generates temporal context information 1606. For example, the base calling system discussed herein may be configured to receive a sample, for which bases are to be called. Such base calling may be performed over a plurality of base calling cycles. In an example, the temporal context information 1606 for the set 1601 of sensor data indicates one or more base calling cycle numbers for which the set 1601 of sensor data is generated. For example, assume that there are N number of base calling cycles and the set 1601 of sensor data is associated with base calling cycles N1 to N2 of the total N base calling cycles. The temporal context information 1606 for the set 1601 of sensor data would include such information. As discussed herein below, the choice of which base caller to be used to process the set 1601 of sensor data can also be based on the base calling cycle number(s) with which the set 1601 of sensor data is associated.

FIG. 17C illustrates an example of fading, in which signal intensity is decreased as a function of cycle number in a sequencing run of a base calling operation. Fading is an exponential decay in fluorescent signal intensity as a function of base calling cycle number. As the sequencing run progresses, the analyte strands are washed excessively, exposed to laser emissions that create reactive species, and subjected to harsh environmental conditions. All of these lead to a gradual loss of fragments in each analyte, decreasing its fluorescent signal intensity. Fading is also called dimming or signal decay. FIG. 17C illustrates one example of fading 1700C. In FIG. 17C, the intensity values of analyte fragments with AC microsatellites show exponential decay.

FIG. 17D conceptually illustrates a decreasing signal-to-noise ratio as cycles of sequencing progress. For example, as sequencing proceeds, accurate base calling becomes increasingly difficult, because signal strength decreases and noise increases, resulting in a substantially decreased signal-to-noise ratio. Physically, it was observed that later synthesis steps attach tags in a different position relative to the sensor than earlier synthesis steps. When the sensor is below a sequence that is being synthesized, signal decay results from attaching tags to strands further away from the sensor in later sequencing steps than in earlier steps. This causes signal decay with the progression of sequencing cycles. In some designs, where the sensor is above the substrate that holds the cluster, the signal could increase, instead of decay, as sequencing proceeds.

In the flow cell design investigated, while the signal decays, noise grows. Physically, phasing and pre-phasing increase noise as sequencing proceeds. Phasing refers to steps in sequencing in which tags fail to advance along the sequence. Pre-phasing refers to sequencing steps in which tags jump two positions forward instead of one, during a sequencing cycle. Phasing and pre-phasing are both relatively infrequent, on the order of once in 500 to 1000 cycles. Phasing is slightly more frequent than pre-phasing. Phasing and pre-phasing impact individual strands in a cluster that is producing intensity data, so the intensity noise distribution from a cluster accumulates in a binomial, trinomial, quadrinomial, etc. expansion as sequencing proceeds.

Further detail of fading, signal decay, and decrease in signal-to-noise ratio, and FIGS. 17C and 17D can be found in U.S. Nonprovisional patent application Ser. No. 16/874,599, titled “Systems and Devices for Characterization and Performance Analysis of Pixel-Based Sequencing,” filed May 14, 2020 (Attorney Docket No. ILLM 1011-4/IP-1750-US), which is incorporated by reference as if fully set forth herein.

Thus, during base calling, a reliability or quality of the balling calling (e.g., a probability of the called base being correct) can be based on a base calling cycle number for which the current base is being called. Thus, a selection of the first base caller 1414 and/or the second base caller 1416 for processing the set 1601 of sensor data can also be based on a current cycle number for which a base calling operation is being performed, which may be included in the temporal context information 1606 for the set 1601 of sensor data, as will be discussed herein later in further detail.

### Base Sequence Context Information 1608

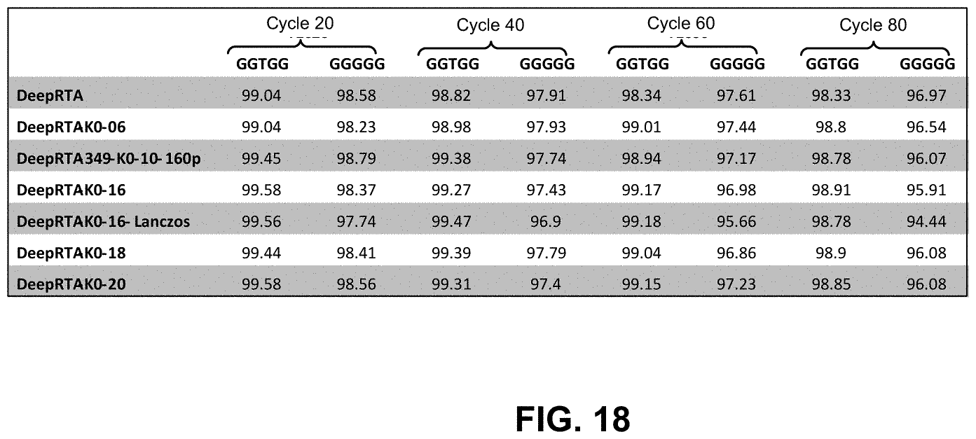

FIG. 18 shows a base calling accuracy (1-base calling error rate) of the different example configurations of base caller (e.g., DeepRTA, DeepRTA-K0-06, DeepRTA-349-K0-10-160p, DeepRTA-K0-16, DeepRTA-K0-16-Lanczos, DeepRTA-K0-18, and DeepRTA-K0-20) over base calling homopolymers (e.g., GGGGG), and sequences with near-homopolymers or sequences with flanking homopolymers (e.g., GGTGG). In an example, sequences with flanking homopolymers (e.g., GGTGG) comprises homopolymers (e.g., GG) flanking on both sides of a base of interest (e.g., T). Similarly, near-homopolymers comprises sequences where most or majority of the bases are the same (e.g., 3 out of 5 bases, or 4 out of 5 bases, or 4 out of 7 bases are G). The table illustrated in FIG. 18 shows data (e.g., base calling probabilities, or probability of calling a base correctly) for various base calling cycles, such as cycles 20, 40, 60, and 80. For example, a probability of correctly calling a middle base of a sequence GGGGG, using a DeepRTA base caller, at cycle 80 is 96.97%. Note that some examples of homopolymers, near-homopolymers or sequences with flanking homopolymers discussed in this disclosure are assumed to have five bases. However, there may be any different number of bases in such special sequences, such as three, five, six, seven, nine, or another appropriate number.

As discussed above, in some implementations, the base caller makes a base call for a current sequencing cycle by processing a window of sequencing images for a plurality of sequencing cycles, including the current sequencing cycle contextualized by right and left sequencing cycles. Since the base “G” is indicated by a dark or minimal signal state (also referred to herein as off state, indecipherable signal state, or inactive state) in the sequencing images, repeat patterns of the base “G” can lead to erroneous base calls. Such erroneous base calls also occur when the current sequencing cycle is for a non-G base (e.g., base “T”), but right and left flanked by Gs. Note that non-G bases (i.e., A, C, or T) are indicated by lighted or on (or active) states in the sequencing images.

In an example, there are some specific base calling sequence patterns for which the probability of error in base calling is relatively high. Two such examples, of GGGGG and GGTGG, are illustrated in FIG. 18. There may be other specific base calling sequence patterns, such as GGTCG, for which probability of error in base calling is also relatively high. In an example, such specific base calling sequence patterns have multiple G's, such as G's at least at a beginning and at an end of the sequence, and possibly a third G between the two end-G's in the 5-base sequence. Other examples of such specific base calling sequences include GGXGG, GXGGG, GGGXG, GXXGG, and GGXXG, where X can be any of A, C, T, or G.

In an example, the base sequence context information 1608 for the set 1601 of sensor data also provides an indication as to whether the set 1601 of sensor data is associated with any such special base sequence pattern. For example, if the set 1601 of sensor data is for calling a middle base of the sequence GGGGG (or GGTGG), then this may necessitate special operations to generate the final base calls, as will be discussed herein (e.g., see FIGS. 19B, 19C, 20A).

### Other Context Information 1610

Referring again to FIG. 16, the context information generation module 1418 further generates other context information 1610. The other context information 1610 may cover any type of context information that is not covered by the spatial, temporal, and base sequence context information. Numerous examples of other context information 1610 are possible, some of which are discussed herein below.

Sometimes bubbles are formed over one or more clusters during one or more sequences of base calling operations. Such a bubble may be a globule of gas (such as air) in any liquid present in the cluster (such as a bubble within the reagents used for base calling). The presence of a bubble can be detected based on analyzing the images captured from the affected cluster(s). For example, the presence of a bubble within a cluster can be estimated by detecting unique intensity signal signature in the captured image of the cluster. In an example, the other context information 1610 may indicate whether the set 1601 of sensor data from a cluster is associated with such a bubble. Put differently, if the images within the set 1601 of sensor data from a cluster indicate presence of a bubble in the cluster, then the other context information 1610 provides an indication of such a bubble in the cluster. Detection of bubbles are discussed in further detail in co-pending U.S. Patent Application No. 63/170,072, titled “MACHINE-LEARNING MODEL FOR DETECTING A BUBBLE WITHIN A NUCLEOTIDE-SAMPLE SLIDE FOR SEQUENCING,” filed Apr. 2, 2021, which is incorporated by reference herein. Further detail about generation of final base calls, in an event of detection of bubbles, is discussed herein later in turn.

In an example, reagents used in the flow cell play a major role in how the base callings are to be performed. For example, the first base caller 1414 may be suitable if a first type of reagent is used, whereas the second base caller 1416 may be suitable if a second type of reagent is used. In an example, the other context information 1610 provides an indication of reagents used in the flow cell. Further detail about generation of final base calls, based on selection of the reagents, is discussed herein later in turn.

Selective Usage of the First Base Caller 1414 and the Second Base Caller 1416: Final Base Call a Function (e.g., Average, Maximum, Minimum, or Another Appropriate Function) of Classification Information from the Two Base Callers

FIG. 19A illustrates generation of a final base call for a set of sensor data, based on a function of the first base call classification information 1434 from the first base caller 1414 and the second base call classification information 1436 from the second base caller 1416 of the system 1400 of FIG. 14.

In an embodiment, each base caller 1414, 1416 outputs corresponding probabilities of the base to be called being an A, C, G, or T. For example, consider the first base call classification information 1434 from the first base caller 1414. The first base call classification information 1434 is, for a given base to be called, in the form of probability or confidence scores p1(A), p1(C), p1(G), p1(T). Here, p1(A) is indicative of the probability that the base to be called is an A; p1(C) is indicative of the probability that the base to be called is a C; p1(G) is indicative of the probability that the base to be called is a G; and p1(T) is indicative of the probability that the base to be called is a T. Merely as an example, if p1(A), p1(C), p1(G), p1(T) are 0.6, 0.2, 0.15, 0.05, respectively, then the first base caller 1414 indicates a high probability of 0.6 that the base to be called is an A.

In an example, a summation of p1(A), p1(C), p1(G), p1(T) is 1. Thus, the first base caller outputs a normalized probability of each base, e.g., using a softmax function in one example. In another example, other techniques (e.g., other than softmax) may be used. For example, the base caller has an output layer that does not use softmax. For example, a regression based operation may be used, which may use, for example, Euclidean or Mahalanobis distance to a cloud center to derive a probability measure for each base.

Similarly, the second base call classification information 1436 is, for a given base to be called, in the form of probability or confidence scores p2(A), p2(C), p2(G), p2(T), where a summation of p2(A), p2(C), p2(G), p2(T) is 1.

In an embodiment, in addition to the probabilities discussed herein above, a base caller may also output a corresponding called base. For example, the first base caller 1414 outputs a first called base, and the second base caller 1416 outputs a second called base.

A simple rule for base calling is as follows. For example, assume the first base call classification information 1434 output by the first base caller 1414, for a given base to be called, is p1(A), p1(C), p1(G), p1(T), where p1(C) is greater than each of p1(A), p1(G), and p1(T). Then, the first base caller 1414 may call the base to be C. In another example, the first base caller 1414 may call the base to be C only if the corresponding probability p1(C) is higher than a threshold probability. In yet another example, assume p1(C)>p1(A)>p1(T) and p1(G). That is, p1(C) has the highest probability, followed by the probability p1(A). Then, the first base caller 1414 may call the base to be C if p1(C) is higher than p1(A) by at least a threshold value (i.e., if the difference between the probabilities for the two bases is at least the threshold amount). Any other appropriate rule(s) for base calling can be envisioned, based on the teachings of this disclosure. The second base caller 1416 may also call the base accordingly.

Referring again to FIG. 19A, the base calling combining module 1428 receives the first base call classification information 1434 and the second base call classification information 1436, and the context information 1420. Assume that based on the context information 1420, the base calling combining module 1428 decides to combine the first and second base call classification information, e.g., as discussed with respect to the method 1500c of FIG. 15C. Thus, in this example, both base callers 1414 and 1416 are processing the set of sensor data, and the final confidence scores pf(A), pf(C), pf(G), pf(T) and the final called base are based on the output of both the base callers 1414 and 1416.

When Classification Information from the Two Base Callers Agree or Match, Use Average (e.g., Arithmetic Mean), Minimum, Maximum, or Geometric Mean of the Confidences Scores from the Two Base Callers

Still referring to FIG. 19A, assume a scenario where the first base call classification information 1434 from first base caller 1414 and the second base call classification information 1436 from second base caller 1416 match. For example, the first base caller 1414 outputs the first base call classification information 1434 that includes the confidence scores of p1(A), p1(C), p1(G), p1(T), and calls the base to be C, merely as an example. Also, for example, the second base caller 1416 outputs the second base call classification information 1436 that includes the confidence scores of p2(A), p2(C), p2(G), p2(T), and also calls the base to be C, merely as an example. Thus, the base calling from both the base callers match and are C in this example.

In such a scenario where the base calls from both base callers 1414 and 1416 match, the final base call 1440 includes the final called base that matches with the base calls made by the base callers 1414 and 1416.

In an embodiment, the final confidence scores pf(A), pf(C), pf(G), pf(T) are an appropriate function of the confidence scores p1(A), p1(C), p1(G), p1(T) output by the first base caller 1414 and the confidence scores p2(A), p2(C), p2(G), p2(T) output by the second base caller 1416.

For example, each of the final confidence scores pf(A), pf(C), pf(G), pf(T) can be an average or arithmetic mean of corresponding ones of the confidence scores p1(A), p1(C), p1(G), p1(T) output by the first base caller 1414 and corresponding ones of the confidence scores p2(A), p2(C), p2(G), p2(T) output by the second base caller 1416. Thus, if both base callers 1414 and 1416 call the base under consideration to be C, then the base calling combining module 1428 outputs the final called base to be C, and outputs the final confidence scores to be:

pf(A)=average of (p1(A) and p2(A)),

pf(C)=average of (p1(C) and p2(C)),

pf(G)=average of (p1(G) and p2(G)), and

pf(T)=average of (p1(T) and p2(T)). Equations 1

In another example, instead of the average or arithmetic mean, another mathematical function (such as geometric mean) can be used. For example, if geometric mean is used, then equations 1 can be rewritten as:

pf(A)=geometric mean of (p1(A) and p2(A))=\sqrt{p1(A)·p2(A)},

pf(C)=geometric mean of (p1(C) and p2(C))=\sqrt{p1(C)·p2(C)},

pf(G)=geometric mean of (p1(G) and p2(G))=\sqrt{p1(G)·p2(G)}, and

pf(T)=geometric mean of (p1(T) and p2(T))=\sqrt{p1(T)·p2(T)}. Equations 1a

In another example, if the base calling system 1400 wants to report a conservative score, then each of the final confidence scores pf(A), pf(C), pf(G), pf(T) can be a minimum of the corresponding ones of the confidence scores p1(A), p1(C), p1(G), p1(T) output by the first base caller 1414 and the corresponding ones of the confidence scores p2(A), p2(C), p2(G), p2(T) output by the second base caller 1416 (e.g., assuming that the base callings of the two base callers match). Thus, if both base callers 1414 and 1416 call the base under consideration to be C, then the base calling combining module 1428 outputs the final called base to be C, and outputs the final confidence scores to be:

pf(A)=minimum of (p1(A) and p2(A)),

pf(C)=minimum of (p1(C) and p2(C)),

pf(G)=minimum of (p1(G) and p2(G)), and

pf(T)=minimum of (p1(T) and p2(T)). Equations 2

In yet another example, if the base calling system 1400 wants to report a high confidence score, then each of the final confidence scores pf(A), pf(C), pf(G), pf(T) can be a maximum of corresponding ones of the confidence scores p1(A), p1(C), p1(G), p1(T) output by the first base caller 1414 and corresponding ones of the confidence scores p2(A), p2(C), p2(G), p2(T) output by the second base caller 1416 (e.g., assuming that the base callings of the two base callers match). Thus, if both base callers 1414 and 1416 call the base under consideration to be C, then the base calling combining module 1428 outputs the final called base to be C, and outputs the final confidence scores to be:

pf(A)=maximum of (p1(A) and p2(A)),

pf(C)=maximum of (p1(C) and p2(C)),

pf(G)=maximum of (p1(G) and p2(G)), and

pf(T)=maximum of (p1(T) and p2(T)). Equations 3

In another example, if the base calling system 1400 wants to report a weighted confidence score, then each of the final confidence scores pf(A), pf(C), pf(G), pf(T) can be a normalized weighted sum of corresponding ones of the confidence scores p1(A), p1(C), p1(G), p1(T) output by the first base caller 1414 and corresponding ones of the confidence scores p2(A), p2(C), p2(G), p2(T) output by the second base caller 1416 (e.g., assuming that the base callings of the two base callers match). Thus, if both base callers 1414 and 1416 call the base under consideration to be C, then the base calling combining module 1428 outputs the final called base to be C, and outputs the final confidence scores to be:

pf(A)=A1×p1(A)+A2×p2(A),

pf(C)=A1×p1(C)+A2×p2(C),

pf(G)=A1×p1(G)+A2×p2(G), and

pf(T)=A1>p1(T)+A2×p2(T). Equations 4

In an example, weights A1 and A2 in equations 4 are fixed, pre-specified weights, such that A1+A2=1. In an example, the weights A1 and A2 are tuned or updated during a training process, e.g., based on training data.

Normalized Ratio of the Confidences Scores from the Two Base Callers, Based on Temporal Context (e.g., Base Calling Cycle Number) Associated with the Sensor Data

In an example, the base calling system 1400 generates each of the final confidence scores pf(A), pf(C), pf(G), pf(T) to be a weighted average of each of the confidence scores p1(A), p1(C), p1(G), p1(T) output by the first base caller 1414 and the corresponding ones of the confidence scores p2(A), p2(C), p2(G), p2(T) output by the second base caller 1416 (e.g., assuming that the base callings of the two base callers match), where the weights are based on the context information 1420. That is, the context information 1420 dictates the weights to be given to individual ones of the confidence scores from the two base callers.

FIG. 19A1 illustrates a Look Up Table (LUT) 1901 indicating an example weightage scheme to be used for the final confidence scores, based on temporal context information 1606 (see FIG. 16). Actual weightages included in the LUT 1910 are mere examples, and are non-limiting.

Due to the phasing, pre-phasing and fading discussed with respect to FIGS. 17C and 17D, it has been observed that performances of both the base callers 1414 and 1416 are comparable during the initial base calling cycles (e.g., see FIG. 17D illustrating relatively better signal quality and less noise during the initial base calling cycles). During the later base calling cycles, the first base caller 1414 outperforms the second base caller 1416, as the first base caller 1414 may be better equipped to handle the signal degradation during the later base calling cycles. However, the first base caller 1414 may be computationally intensive to operate, compared to the operations of the second base caller 1416.

Accordingly, in an example and as illustrated in FIG. 19A1, confidence scores from the second base caller 1416 are emphasized over confidence scores from the first base caller 1414 during an initial threshold number of base calling cycles. As and when the base calling cycles progress, more emphasis is placed on the confidence scores from the first base caller 1414 (e.g., because the first base caller 1414 outperforms the second base caller 1414 during later cycles).

Specifically, referring to the first row of the LUT 1901, assume there are N number of base calling cycles. For base calling cycles 1−N1 (i.e., initial N1 number of base calling cycles), high (e.g., 90-100%) weightage is given to confidence scores from the second base caller 1416, and low (e.g., 0-10%) weightage is given to confidence scores from the first base caller 1414. Thus, during the base calling cycles 1−N1, the first base caller 1414 may be disabled or non-operational. As the first base caller 1414 is computationally intensive to operate (e.g., compared to operations of the second base caller 1416), this increases computation efficiency. As discussed, during the first N1 cycles, both base callers have comparable performance, and hence, no degradation in a quality of base calling is observed.

Here N1 is an appropriate number of base calling cycles that is between 1 and N2 (discussed later). Merely as an example, N1 can be 100, 150, 200, 250 or another appropriate number of base calling cycles. N1 may be determined to be a number of initial base calling cycles for which both the base callers provide a reasonably comparable quality of base calls.

Thus, for example, for a base calling cycle between cycles 1 and N1, a final base call pf is given by (e.g., assuming 100% weightage given to second base caller):

for cycles 1 to N1: pf(A)=p2(A); pf(C)=p2(C); pf(T)=p2(T); and pf(G)=p2(G). Equation 5

As discussed, the first base caller 1414 can be disabled (i.e., not operating on the corresponding set of data) for at least an initial N1 cycles. Note that the first base caller 1414 is to operate on a corresponding set of data from cycle (N1+1) onwards (see the second row of the LUT 1901). To satisfactorily generate base calls for cycle (N1+1) and subsequent cycles, the first base caller 1414 has to also operate at least a few cycles before the cycle (N1+1), e.g., because, as discussed with respect to FIGS. 7 and 10, the base call for a current cycle is also based on data from one or more past cycles and one or more future cycles (also see discussion with respect to FIG. 15E for further explanation). Accordingly, the first base caller 1414 can be nonoperational on the corresponding set of data between cycle 1 and cycle (N1−T), and operational from cycle (N1−T+1). There is a threshold number of cycles, the data from which are needed to start base calling from cycle (N1+1).

Now referring to the second row of the LUT 1901, in an example, for base calling cycles (N1+1) to N2, a first weightage is given to confidence scores from the second base caller 1416 and a second weightage is given to confidence scores from the first base caller 1414. In the example of FIG. 19A1, both the first and second weightages are medium weights, such as about 50%, merely as an example. Thus, during these cycles, both the base callers 1414 and 1416 operate. Thus, for example, for base calling cycles between cycles N1+1 and N2, final scores pf are given by:

for cycles (N1+1) to N2:

pf(A)=0.5×p1(A)+0.5×p2(A);

pf(C)=0.5×p1(C)+0.5×p2(C);

pf(G)=0.5×p1(G)+0.5×p2(G); and

pf(T)=0.5×p1(T)+0.5×p2(T). Equations 6

Now referring to the third row of the LUT 1901, in an example, for base calling cycles (N2+1) to N, low (e.g., 0%) weightage is given to confidence scores from second base caller 1416, and high (e.g., 100%) weightage is given to confidence scores from first base caller 1414. This is because, as discussed herein, during the later base calling cycles, the first base caller 1414 outperforms the second base caller 1416, as the first base caller 1414 may be better equipped to handle the signal degradation (see FIGS. 17C, 17D) during the later base calling cycles.

Thus, for example, for base calling cycles between cycles (N2+1) and N, final scores pf are given by:

for cycles (N2+1) to N:

pf(A)=p2(A);

pf(C)=p2(C);

pf(T)=p2(T); and

pf(G)=p2(G). Equations 7

In an example, the LUT 1901 (or any other LUTs discussed herein) may be saved in a memory of the system 1400 (the memory not illustrated in FIG. 14). The switching module 1422 and/or the base calling combining module 1428 access the LUT 1901 from the memory, and receive context information 1420 (e.g., temporal context information) that is indicative of the current base calling cycle number. Based on the temporal context information, the switching module 1422 and/or the base calling combining module 1428 select an appropriate row of the LUT 1901, and operate in accordance with the weights specified in the selected row.

Confidence Score Correction, Based on Base Sequence Context Information Indicating Special Base Sequence (with Base Calls from Two Base Callers Matching)

Assume a scenario where the base callings from the two base callers match, and the base sequence context information indicates that the called bases from any of the base callers include a special base sequence, such as a homopolymer (e.g., GGGGG), a sequence with flanking homopolymer (e.g., GGTGG), near homopolymer, or another special base sequence. In an example, five consecutive final base calls are made by the base calling combining module 1428 (or any of the base callers 1414, 1416), and the five consecutive final base calls turn out to include the special base sequence. As discussed herein previously (see FIG. 18), probability of error for such special base sequences may be higher. Accordingly, the system 1400 may undertake special measure to possibly modify the confidence scores associated with bases of such base sequences Again, note that some examples of special base sequences (such as homopolymers, near-homopolymers, or sequences with flanking homopolymers) discussed in this disclosure have five bases. However, there may be any different number of bases in such special base sequences, such as three, five, six, seven, nine, or another appropriate number.

FIG. 19B illustrates a LUT 1905 indicating a base caller to be used when the called bases include a special base sequence. In the LUT 1905, the letter “X” indicates any base, such as A, C, T, or G. Thus, for any of the base sequences included in the LUT 1905 (such as GGXGG, GXGGG, GGGXG, GXXGG, GGXXG), confidence scores from the second base caller 1416, for example, are to be used to determine the final confidence scores. This is because it has been determined, through experimentations by the inventors, that the second base caller 1416 outperforms the first base caller 1414 when encountering any of the special base sequences indicated in the LUT 1905.

Thus, if five consecutive final base calls made by the base calling combining module 1428 are any of the special base sequences of the LUT 1905, the base calling combining module 1428 modifies the confidence scores associated with each (or at least some, such as the middle one) of the five bases to correspond to the confidence scores output by the second base caller 1416 for the five bases.

In an example, the LUT 1905 (or any other LUTs discussed herein) may be saved in a memory of the system 1400 (the memory not illustrated in FIG. 14). The switching module 1422 and/or the base calling combining module 1428 access the LUT from the memory, and receive the context information 1420. Based on the context information, the switching module 1422 and/or the base calling combining module 1428 select an appropriate row of the LUT, and operate in accordance with the base calling operations specified in the selected row. Unless otherwise specified, this applies to of all the LUTs discussed in this disclosure.

FIG. 19C illustrates a LUT 1910 indicating weightages given to confidence scores of individual base callers, when the called bases include a special base sequence. Note that the actual weightages included in the LUT 1910 are mere examples and do not limit the scope of this disclosure. For example, referring to the first row of the LUT 1910, if a special sequence GGXGG is encountered, 60% weightage may be given to confidence scores from the second base caller 1416, and 40% weightage may be given to confidence scores from the first base caller 1414. For example, assume that the middle (i.e., 3^{rd}) base of the sequence indicated by “X” is a T, with the first base caller 1414 indicating a confidence score of p1(T) and the second base caller 1414 indicating a confidence score of p2(T). In such an example, the final called base is a T and the final confidence scores for the middle (i.e., 3^{rd}) base of the sequence are:

pf(A)=0.6×p1(A)+0.4×p2(A),

pf(C)=0.6×p1(C)+0.4×p2(C),

pf(G)=0.6×p1(G)+0.4×p2(G), and

pf(T)=0.6×p1(T)+0.4×p2(T). Equations 8

In an example, the weights in the LUT 1910 can be empirically determined through testing and calibration.

Other rows of the LUT 1910 can also have weights that are based on the detected base sequences. These weights can be pre-specified, and optimal values of these weightages can be determined via testing and calibration.

Note that in all of the example weights specified in the LUT 1910, the weights for the second base caller 1416 are higher than the weights for the first base caller 1414. This is because, as discussed herein above, in some examples, the second base caller 1416 may outperform the first base caller 1414 when encountering any of the special base sequences indicated in the LUT.

### Generation of Final Classification Information, Based on Bubble Detection in a Cluster

As previously discussed herein, sometimes bubbles are formed over one or more clusters during one or more sequences of base calling operations. Such a bubble may be a globule of gas (such as air) in any liquid present in the cluster (such as a bubble within the reagents used for base calling). Presence of a bubble can be detected based on analyzing the images captured from the affected cluster(s). For example, a presence of a bubble within a cluster can be estimated by detecting a unique intensity signal signature in the captured image of the cluster. In an example, the other context information 1610 may indicate whether the set 1601 of sensor data from a cluster is associated with such a bubble.

FIG. 19D illustrates a LUT 1915 indicating operation of the base calling combining module 1428 of FIG. 14, in view of detection of one or more bubbles in a cluster of the flow cell. For example, referring to the first row of the LUT 1915, if bubbles are not detected in the flow cell, the final base calling is performed by the base calling combining module 1428 normally, e.g., in accordance with any appropriate operational scheme discussed herein in this disclosure.

Referring to the second row of the LUT 1915, a scenario is discussed where one or more bubbles are detected in a cluster of the flow cell. Generally, the first base caller 1414 is better equipped to handle base calls for clusters including such bubbles. So, in an embodiment, in response to the other context information 1610 (see FIG. 16) indicating presence of a bubble in the cluster, the base calling combining module 1428 places relatively higher weightage (e.g., 90˜100% weightage) on the confidence scores from the first base caller 1414, and relatively lower weightage (e.g., 0˜10% weightage) on confidence scores from the second base caller 1416.

Note that a tile of the flow cell comprises a plurality of clusters, and a bubble is detected in, for example, a single cluster of the tile. So, sensor data from the single cluster are processed primarily by the first base caller 1414 in accordance with the second row of the LUT 1915, while sensor data from other clusters of the tile are processed by the first base caller 1414 and/or the second base caller 1416 in accordance with the first row of the LUT 1915.

Assume no bubbles were detected in a cluster for base calling cycles 1 to Na, and at cycle (Na+1) the cluster was detected to include a bubble. So, from base calling cycle (Na+1) onwards, the first base caller 1414 is to process sensor data from the cluster, in accordance with the second row of the LUT 1915. However, assume that prior to cycle (Na+1) (i.e., from cycles 1 to Na), the first base caller 1414 was non-operational on the sensor data from that cluster, and the second base caller 1416 was operational on the sensor data from that cluster. But for the first base caller 1414 to start calling bases from cluster (Na+1), the first base caller 1414 has to also process past few cycles (e.g., because, as discussed with respect to FIGS. 7 and 10, base call for a current cycle is also based on data from one or more past cycles and one or more future cycles; also see discussion with respect to FIG. 15E). Accordingly, in response to the context information indicating presence of a bubble at cycle (Na+1), the first base caller processes sensor data for a few cycles that occur prior to cycle (Na+1) (e.g., processes sensor data for cycles Na, cycle (Na−1), cycle (Na−2), (Na−T)), and based on processing such past cycles, is now prepared to process and base call the cycle at (Na+1). T is a threshold number of past base calling cycles that the first base caller has to process, to correctly process sensor data for a current base calling cycle.

### Generation of Final Classification Information, Based on Out-of-Focus Even Detection in a Cluster

As previously discussed herein, the flow cell 1405 may include a lens (such as the filter layer 124 including an array of microlenses or other optical components) for capturing images of various clusters. In an example, there may be slight differences in focusing for various clusters, when capturing the images, e.g., as the image sensor or camera is moved around the flow cell. For example, edge clusters 1407a may be slightly out of focus, relative to the non-edge clusters 1407b, when images of the clusters are captured. Out of focus events may also occur due to heating or mechanical vibration cause by movement of the lens.

FIG. 19D1 illustrates a LUT 1917 indicating operation of the base calling combining module 1428 of FIG. 14, in view of detection of out of focus image(s) from a cluster of the flow cell. For example, referring to the first row of the LUT 1917, if out of focus images are not detected for the flow cell, the final base calling is performed by the base calling combining module 1428 normally, e.g., in accordance with any appropriate operational scheme discussed herein in this disclosure.

Referring to the second row of the LUT 1917, a scenario is discussed where an out of focus image(s) is detected from one or more clusters of the flow cell. Generally, the first base caller 1414 is better equipped to handle base calls for clusters generating such out of focus images. So, in an embodiment, in response to the other context information 1610 (see FIG. 16) indicating presence of out of focus images from the cluster, the base calling combining module 1428 places relatively higher weightage (e.g., 90˜100% weightage) on the confidence scores from the first base caller 1414, and relatively lower weightage (e.g., 0˜10% weightage) on confidence scores from the second base caller 1416.

Note that a tile of the flow cell comprises a plurality of clusters, and the out of focus images may be detected in, for example, a single cluster or a few clusters (but not all or even a majority of the clusters) of the tile. So, sensor data from the one or more clusters having the out of focus images are processed primarily by the first base caller 1414 in accordance with the second row of the LUT 1915, while sensor data from the other clusters of the tile are processed by the first base caller 1414 and/or the second base caller 1416 in accordance with the first row of the LUT 1915.

Normalized Ratio of the Confidences Scores from the Two Base Callers, Based on Reagents Used

Reagents play a major role in base calling, as discussed herein earlier. Merely as an example, the first base caller 1414 may be more suitable than the second base caller 1416 when a first group of reagents is used, and the first base caller 1414 may be less suitable than the second base caller 1416 when a second group of reagents is used. In an embodiment, the context information 1601 indicates the types of reagents used, and the context information generation module 1418 can specify normalized weightages for the confidences scores from the two base callers for determining the final confidence scores.

FIG. 19E illustrates a LUT 1920 indicating example weightages given to confidence scores of individual base callers, based on groups of reagents used. For example, referring to the first row of the LUT 1920, when an example reagent group A is used, A1% weightage is given to confidence scores from the first base caller 1414 and A2% weightage is given to confidence scores from the second base caller 1416, where A1+A2=100. Similarly, referring to the second row of the LUT 1920, when an example reagent group B is used, B1% weightage is given to confidence scores from the first base caller 1414 and B2% weightage is given to confidence scores from the second base caller 1416, where B1+B2=100.

Normalized Ratio of the Confidences Scores from the Two Base Callers, in Logarithmic Probability Domain

Various examples and embodiments herein above discuss confidence scores in terms of probability. However, in an embodiment, the confidence scores can be expressed using a logarithmic scale, and mathematical operations discussed herein (e.g., with respect to equations 1-8) can be performed with the confidence scores expressed using the logarithmic scale. For example, Phred quality score is a measure of the quality of the identification of the nucleobases generated by automated DNA sequencing. Phred quality scores Q are defined as a property which is logarithmically related to the base-calling probabilities P as follows:

Q=−10×log₁₀P. Equation 9

Thus, a 90% base calling accuracy (e.g., p1(c) having a value of 0.9) translates to a corresponding Phred score of 10, a 99% base calling accuracy (e.g., p1(c) having a value of 0.99) translates to a corresponding Phred score of 20, and so on. Here, P is the base calling probability, which is related to a probability of error E as follows: P=(1−E). Thus, the Phred quality score Q is related to the probability of error E as follows: Q=−10×log₁₀(1−E), where E is a probability of error of a specific base call. Additional detail of quality scores and probability of errors are discussed in, for example, co-pending U.S. Provisional Patent Application No. 63/226,707 (Attorney Docket Number (ILLM 1045-1/IP-2093-PRV)), titled “QUALITY SCORE CALIBRATION OF BASECALLING SYSTEMS,” filed on Jul. 28, 2021, which is incorporated by reference.

In an embodiment, the mathematical operations discussed herein (e.g., with respect to equations 1-8) can be performed using the Phred score, instead of the confidence scores. Thus, in some examples where the mathematical operations use the Phred or quality scores, the selection of the base caller to be used can be based on the Phred or quality scores (e.g., as discussed with respect to equations 1-8).

Generation of Final Confidences Scores from the Two Base Callers, Based on Spatial Context, e.g., Edge Tiles, Associated with the Sensor Data

As previously discussed with respect to FIG. 17A, some tiles may be classified as edge tiles, based on the spatial location of the tiles. For example, in FIG. 17A, tiles that are adjacent to any edge of the flow cell 1405 are labeled as edge tiles 1406a, and the remaining tiles are labeled as non-edge tiles 1406b. For example, tiles that are on vertical (e.g., along the Y-axis) and/or horizontal (e.g., along the X-axis) edges of the flow cell 1404 are categorized as edge tiles 1406, as illustrated in FIG. 14. Thus, an edge tile 1406 is immediate adjacent to a corresponding edge of the flow cell 1404.

As also discussed with respect to FIG. 17A, in an example, parameters relevant for base calling operations for a tile can be based on a relative location of the tile. For example, the excitation light 101 discussed with respect to FIG. 1 is directed towards the tiles of the flow cell, and different tiles can receive different amount of the excitation light 101, e.g., based on the location of an individual tile and/or the location of one or more light sources emitting the excitation light 101. For example, if the light source(s) emitting the excitation light 101 is vertically above the flow cell, non-edge tiles 1406b may receive a different amount of light than the edge tiles 1406a. In another example, peripheral or external light (e.g., ambient light from outside the bio sensor) around the flow cell 1405 can influence the amount and/or characteristics of the excitation light 101 received by individual tiles of the flow cell 1405. Merely as an example, the edge tiles 1406a may receive the excitation light 101, along with some amount of peripheral light from outside the flow cell 1405, whereas the non-edge tiles 1406b may mainly receive the excitation light 101. In yet another example, individual sensors (or pixels or photodiodes) included in the flow cell 1405 (e.g., sensors 106, 108, 110, 112, and 114 illustrated in FIG. 1) can sense light based on the locations of the corresponding sensors, which are based on the locations of the corresponding tiles. For example, sensing operations performed by one or more sensors associated with the edge tiles 1406a can be influenced by peripheral light (along with the excitation light 101) relatively more than an effect of the peripheral light on sensing operations of one or more other sensors associated with the non-edge tiles 1406b. In yet another example, the flow of the reactant (e.g., which includes any substance that may be used to obtain a desired reaction during the base calling, such as reagents, enzymes, samples, other biomolecules, and buffer solutions) to various tiles can also be influenced by a tile location. For example, tiles near a source of the reactants can receive higher amounts of reactants than tiles that are further from the source.

In an example, spatial context information 1604 associated with the set 1601 of sensor data (see FIG. 16) includes information regarding whether the set 1601 of sensor data was generated in an edge tile 1406a or a non-edge tile 1406b.

FIG. 19F illustrates a LUT 1925 indicating operation of the base calling combining module 1428 of FIG. 14, in view of spatial classification of the tiles. For example, referring to the first row of the LUT 1925, for non- edge tiles, the final base calling is performed by the base calling combining module 1428 normally, e.g., in accordance with any appropriate operational scheme discussed herein in this disclosure.

Referring to the second row of the LUT 1925, a scenario is discussed for final base calling for edge tiles. Generally, as discussed herein, the first base caller 1414 is better equipped to handle base calls for edge tiles. So, in an embodiment, for the edge tiles, the base calling combining module 1428 places E1 weightage on the confidence scores from the first base caller 1414 and E2 weightage on the confidence scores from the second base caller 1416, where in an example, E1 is higher than E2, and a sum of E1 and E2 is 100% (i.e., the weightages are normalized).

Generation of Final Confidences Scores from the Two Base Callers, Based on Spatial Context, e.g., Edge Clusters, Associated with the Sensor Data

As previously discussed with respect to FIG. 17B, the clusters 1407 of the example tile 1406 are categorized as either edge clusters 1407a or non-edge clusters 1407b. As also discussed herein previously, the flow cell 1405 may include a lens (such as the filter layer 124 including an array of microlenses or other optical components) for capturing images of various clusters, and edge clusters 1407a may be slightly out of focus, relative to the non-edge clusters 1407b, when images of the clusters are captured. Thus, depending on the implementation, one of the first or second base callers 1414 or 1416 may be better suited to process sensor data from edge clusters 1407a, while another of the first or second base callers 1414 or 1416 may be better suited to process sensor data from non-edge clusters 1407b. In an example, spatial context information 1604 associated with the set 1601 of sensor data (see FIG. 16) includes information regarding whether the set 1601 of sensor data was generated from one or more edge clusters 1407a or one or more non-edge clusters 1407b, based on which the set 1601 of sensor data can be processed by a specific one, or both the first or second base callers 1414 or 1416.

FIG. 19G illustrates a LUT 1930 indicating operation of the base calling combining module 1428 of FIG. 14, in view of spatial classification of the clusters. For example, referring to the first row of the LUT 1930, for non-edge clusters, the final base calling is performed by the base calling combining module 1428 normally, e.g., in accordance with any appropriate operational scheme discussed herein in this disclosure.

Referring to the second row of the LUT 1930, a scenario is discussed for final base calling for edge clusters. Generally, as discussed herein, the first base caller 1414 may be better equipped to handle base calls for edge tiles. So, in an embodiment, for the edge clusters, the base calling combining module 1428 places C1 weightage on the confidence scores from the first base caller 1414 and C2 weightage on the confidence scores from the second base caller 1416, where in an example, C1 is higher than C2, and the sum of C1 and C2 is 100% (i.e., the weightages are normalized). In an example, the weightage C1 can be as high as 100%, in which case the classification information from the first base caller 1414 is exclusively used for base calling the edge clusters.

When Classification Information from the Two Base Callers Disagree or Do Not Match, Lowering of the Final Confidence Score

As discussed with respect to FIG. 19A, the first base caller 1414 outputs the first called base and first confidence scores of p1(A), p1(C), p1(G), p1(T); and the second base caller 1416 outputs the second called base and second confidence scores of p2(A), p2(C), p2(G), p2(T). In an example, for a given base, the first called base from the first base caller 1414 may not match with the second called base from the second base caller 1416.

For example, assume that the first base caller 1414 calls the base to be A with a confidence score of p1(A) and the second base caller 1416 calls the base to be C with a confidence score of p2(C). In such a scenario, the final called base output by the base calling combining module 1428 is:

Final called base=A, if p1(A) is higher than p2(C); or

C, if p1(A) is lower than p2(C). Equation 10

Note that because the two base calls from the two base callers disagree, there is a high probability of error. Accordingly, the final confidence score can be reduced. For example, assume p1(A) is higher than p2(C) (i.e., p1(A)>p2(C)), and the final called base is A. Then the final confidence score pf(A) corresponding to A is:

pf(A)=lower of (p1(A) and p2(A)). Equation 11

Thus, the final confidence score is artificially lowered, owing to the disagreement in the called base by the two base callers.

In another example, the final confidence score pf(A) is reduced as follows:

pf(A)=W1×p1(A), where weight W1 is less than one. Equation 12

Thus, the final confidence score is lowered using an appropriate weight W1 that is less than one, due to the disagreement in the called base by the two base callers.

When Classification information from the Two Base Callers Disagrees or Does Not Match, Along with Specific Context Information (Such as Special Base Sequence)

As discussed herein above, the first base caller 1414 outputs the first called base and first confidence scores of p1(A), p1(C), p1(G), p1(T); and the second base caller 1416 outputs the second called base and second confidence scores of p2(A), p2(C), p2(G), p2(T). In an example, for a given base, the first called base from the first base caller 1414 may not match with the second called base from the second base caller 1416.

In an embodiment, if the first called base from the first base caller 1414 does not match with the second called base from the second base caller 1416 and such mismatch is also accompanied by one or more specific context information, then the context information can be taken into account for the final called base.

FIG. 20A illustrates a LUT 2000 indicating operation of the base calling combining module 1428 of FIG. 14 when (i) a special base sequence is detected and (ii) the first called base from the first base caller 1414 does not match with the second called base from the second base caller 1416. For example, referring to the first row of the LUT 2000, a scenario is discussed where the first called base from the first base caller 1414 matches with the second called base from the second base caller 1416, and a special base sequence, such as a homopolymer (e.g., GGGGG), a sequence with flanking homopolymer, or near homopolymer (such as GGXGG), is detected. Further examples of such special sequences are discussed with respect to FIG. 19B. Because the first called base from the first base caller 1414 matches with the second called base from the second base caller 1416, the final called base will match with the called base from the first and second base callers, and confidence scores can be calculated in accordance with FIG. 19B and/or in accordance with any appropriate operational scheme discussed herein. As also noted previously, note that some examples of special base sequences (such as homopolymers, near-homopolymers, or sequences with flanking homopolymers) discussed in this disclosure have five bases. However, there may be any different number of bases in such special base sequences, such as three, five, six, seven, nine, or another appropriate number.

Referring now to the second row of the LUT 2000, a scenario is discussed where the first called base from the first base caller 1414 does not match with the second called base from the second base caller 1416, and a special base sequence, such as a homopolymer (e.g., GGGGG), a sequence with flanking homopolymer, or a near homopolymer (such as GGXGG), is detected. Further examples of such special sequences are discussed with respect to FIG. 19B. Because the first called base from the first base caller 1414 does not match with the second called base from the second base caller 1416, the final called base is based on the second called base from the second base caller 1416 (e.g., for the reasons discussed with respect to FIGS. 19B and 19C, the second base caller 1416 is more reliable for such special base sequence). The confidence score for the final called base, for example, can be the minimum or average (or another appropriate function) of the corresponding confidence scores from the two base callers.

When Classification Information from the Two Base Callers Disagrees or Does Not Match, Along with Specific Context Information (Such as Bubble Detection)

FIG. 20B illustrates a LUT 2005 indicating operation of the base calling combining module 1428 of FIG. 14 when (i) a bubble is detected in a cluster and (ii) the first called base from the first base caller 1414 does not match with the second called base from the second base caller 1416.

For example, referring to the first row of the LUT 2005, a scenario is discussed where the first called base from the first base caller 1414 matches with the second called base from the second base caller 1416, and no bubbles are detected in any cluster. Accordingly, the final base call is performed in accordance with any appropriate operational scheme discussed herein.

Referring now to the second row of the LUT 2005, discussed is a scenario where (i) the first called base from the first base caller 1414 does not match with the second called base from the second base caller 1416, and (ii) bubbles are detected in a cluster. Because the first called base from the first base caller 1414 does not match with the second called base from the second base caller 1416, the final called base is based on the first called base from the first base caller 1414 (e.g., for the reasons discussed with respect to FIG. 19D, the first base caller 1414 is more reliable in an event of bubble detection). The confidence score for the final called base, for example, can be the minimum or average (or another appropriate function) of the corresponding confidence scores from the two base callers.

When Classification Information from the Two Base Callers Disagrees or Does Mot Match, Along with Specific Context Information (Such as Out of Focus Images)

FIG. 20C illustrates a LUT 2010 indicating operation of the base calling combining module 1428 of FIG. 14 when (i) one or more out of focus images are detected from at least one cluster and (ii) the first called base from the first base caller 1414 does not match with the second called base from the second base caller 1416.

For example, referring to the first row of the LUT 2010, a scenario is discussed where the first called base from the first base caller 1414 matches with the second called base from the second base caller 1416, and out of focus images are not detected in any cluster. Accordingly, the final base call is performed in accordance with any appropriate operational scheme discussed herein.

Referring now to the second row of the LUT 2010, a scenario is discussed where (i) the first called base from the first base caller 1414 does not match with the second called base from the second base caller 1416, and (ii) one or more out of focus images are detected from at least one cluster. Because the first called base from the first base caller 1414 does not match with the second called base from the second base caller 1416, the final called base is based on the first called base from the first base caller 1414 (e.g., for the reasons discussed with respect to FIG. 19D1, the first base caller 1414 is more reliable in an event of out of focus image detection). The confidence score for the final called base, for example, can be the minimum or average (or another appropriate function) of the corresponding confidence scores from the two base callers.

When Classification Information from the Two Base Callers Disagrees or Does Not Match, Along with Specific Context Information (Such as Spatial Context Information Indicating an Edge Cluster)

FIG. 20D illustrates a LUT 2015 indicating operation of the base calling combining module 1428 of FIG. 14 when (i) the sensor data is from an edge cluster and (ii) the first called base from the first base caller 1414 does not match with the second called base from the second base caller 1416.

For example, referring to the first row of the LUT 2015, a scenario is discussed where the first called base from the first base caller 1414 matches with the second called base from the second base caller 1416, and the set of sensor data is from an edge cluster. Accordingly, the final base call is performed in accordance with any appropriate operational scheme discussed herein, such as that discussed with respect to FIG. 19G.

Referring now to the second row of the LUT 2015, a scenario is discussed where (i) the first called base from the first base caller 1414 does not match with the second called base from the second base caller 1416, and (ii) the sensor data is from an edge cluster. Because the first called base from the first base caller 1414 does not match with the second called base from the second base caller 1416, the final called base is based on the first called base from the first base caller 1414 (e.g., for the reasons discussed with respect to FIG. 19G, the first base caller 1414 is more reliable for edge clusters). The confidence score for the final called base, for example, can be the minimum or average (or another appropriate function) of the corresponding confidence scores from the two base callers.

Detection of Potential Unreliable Confidence Scores, and Selective Switching between Base Callers Based on Such a Detection

As discussed herein with respect to FIGS. 19B and 19C, the first base caller 1414 may not perform satisfactorily (e.g., relative to the second base caller) for some specific detected base sequences, e.g., when calling homopolymers (e.g., GGGGG), sequences with flanking homopolymers, or near homopolymers (e.g., GGTGG). In an embodiment, for some such sequences, the first base caller 1414 may generate high confidence scores for its called bases, although such high confidence scores may be higher than a true confidence for the called bases. Thus, for example, when such relatively high confidence scores (e.g., higher than a threshold value) are called by the first base caller 1414 for homopolymers, or sequences with flanking homopolymers or near homopolymers, such high confidence scores may be unreliable. In some such scenarios, confidence scores from the second base caller 1416 may be used.

In an example, for homopolymers, sequences with flanking homopolymers, or near homopolymers, the confidence scores p1(A), p1(C), p1(G), p1(T) associated with the middle or third called base of sequence may be changed. For example, assume for a sequence with flanking homopolymer (e.g., GGTGG), the first base caller 1414 called the third base with certain confidence scores, where the confidence score for the third base to be a T is relatively high (e.g., higher than a threshold). Accordingly, confidence scores p2(A), p2(C), p2(G), p2(T) from the second base caller 1416 can be used for the third base of the 5-base sequence with flanking homopolymer or near homopolymer, and may be used to determine the final confidence score.

When Classification Information from the Two Base Callers Disagrees or Does Not Match, Final Base Calls to Include Inconclusive Base Call

In an example, when classification information from the two base callers disagrees or does not match, the final base calls 1440 may include inconclusive base calls and corresponding confidence scores. For example, the final confidence scores for various bases can be generated using any of the approaches discussed herein, such as minimum, average, maximum, or a normalized weighted confidence score, and the final base call can be indicated as being inconclusive.

For example, assume that the first base call classification information 1434 includes, for a given base to be called, confidence scores p1(A), p1(C), p1(G), p1(T) and a first called base of A (e.g., because p1(A) is higher than each of p1(C), p1(G), p1(T)). Also assume that the second base call classification information 1436 includes, for the given base to be called, confidence scores p2(A), p2(C), p2(G), p2(T) and a second called base of C (e.g., because p2(C) is higher than each of p2(A), p2(G), p2(T)). As the two base calls do not match, the final base call is an “N,” where in an example “N” denotes an inconclusive base call. In another example and for the specific use case discussed herein, “N” may denote either of the bases A or C (i.e., the first and second base calls output by the two base callers). The final base call N can be appended with the final confidence scores, which may be calculated using any of the equations 1-8 discussed herein earlier.

### Neural Network Based Final Base Call Determination Module

FIG. 21 illustrates a base calling system 2100 comprising a plurality of base callers, to predict base calls of an unknown analyte comprising base sequences, where a neural network-based final base call determination module 2128 determines the final base calls 1440 based on outputs of one or more of the plurality of base callers. In an example, the final base call determination module 2128 takes into account context information and other variables in account (e.g., as discussed with respect to FIG. 19A), to determine how to combine the first base call classification information 1434 and second base call classification information 1436, to generate the final base calls 1440. The system 2100 is at least in part similar to the system 1400 of FIG. 14. However, the context information generation module 1418 and the base calling combining module 1428 of FIG. 14 are replaced by the final base call determination module 2128 in the system 2100 of FIG. 21.

In an example, the final base call determination module 2128 is a neural network-based module that has been trained using outputs from the two base callers 1414 and 1416. The trained final base call determination module 2128 is then used for base calling. The training of the final base call determination module 2128 can be based on one or more final base calling determination operations discussed herein. The operation of the system 2100 of FIG. 21 will be apparent based on the discussion with respect to FIG. 14 and further discussion on final base call determination presented herein. In other examples, the final base call determination module 2128 can be another appropriate machine learning model, such as logistic regression model, gradient boosted trees model, random forest model, Naïve Bayes model, or the like. In an example, the final base call determination module 2128 can be any appropriate machine learning model that can combine the two classification scores, to generate the final base calls 1440.

### Weight Estimation

Various weights have been discussed herein throughout the disclosure, where the weights are used to weigh the first classification information 1434 and the second classification information 1436 while generating the final classification information. Various techniques can be employed for generating the weights.

In an example, the trained neural network model of the final base call determination module 2128 of FIG. 21 can be used to fine-tune the weights. In another example, the weights can also be determined empirically, using a trial and error method or another appropriate method. In yet another example, a prediction covariance matrix of confidence scores can be empirically estimated and used to estimate the weights.

### Base Calling System Architecture

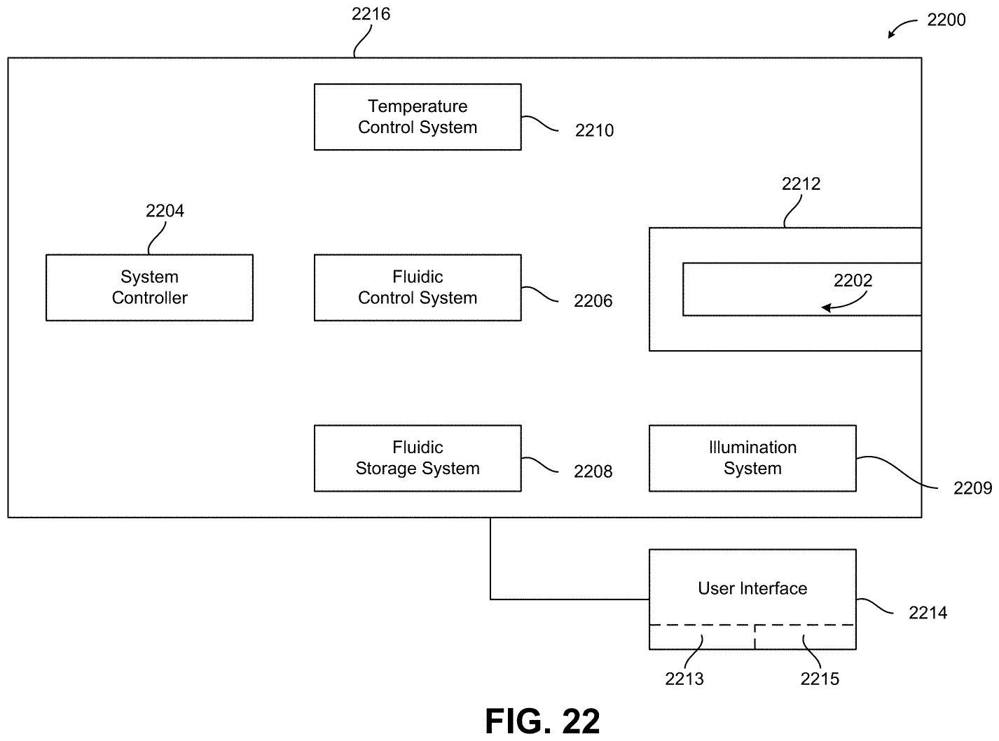

FIG. 22 is a block diagram of a base calling system 2200 in accordance with one implementation. The base calling system 2200 may operate to obtain any information or data that relates to at least one of a biological or chemical substance. In some implementations, the base calling system 2200 is a workstation that may be similar to a bench-top device or desktop computer. For example, a majority (or all) of the systems and components for conducting the desired reactions can be within a common housing 2216.

In particular implementations, the base calling system 2200 is a nucleic acid sequencing system (or sequencer) configured for various applications, including but not limited to de novo sequencing, resequencing of whole genomes or target genomic regions, and metagenomics. The sequencer may also be used for DNA or RNA analysis. In some implementations, the base calling system 2200 may also be configured to generate reaction sites in a biosensor. For example, the base calling system 2200 may be configured to receive a sample and generate surface attached clusters of clonally amplified nucleic acids derived from the sample. Each cluster may constitute or be part of a reaction site in the biosensor.

The exemplary base calling system 2200 may include a system receptacle or interface 2212 that is configured to interact with a biosensor 2202 to perform desired reactions within the biosensor 2202. In the following description with respect to FIG. 22, the biosensor 2202 is loaded into the system receptacle 2212. However, it is understood that a cartridge that includes the biosensor 2202 may be inserted into the system receptacle 2212 and in some states the cartridge can be removed temporarily or permanently. As described above, the cartridge may include, among other things, fluidic control and fluidic storage components.

In particular implementations, the base calling system 2200 is configured to perform a large number of parallel reactions within the biosensor 2202. The biosensor 2202 includes one or more reaction sites where desired reactions can occur. The reaction sites may be, for example, immobilized to a solid surface of the biosensor or immobilized to beads (or other movable substrates) that are located within corresponding reaction chambers of the biosensor. The reaction sites can include, for example, clusters of clonally amplified nucleic acids. The biosensor 2202 may include a solid-state imaging device (e.g., CCD or CMOS imager) and a flow cell mounted thereto. The flow cell may include one or more flow channels that receive a solution from the base calling system 2200 and direct the solution toward the reaction sites. Optionally, the biosensor 2202 can be configured to engage a thermal element for transferring thermal energy into or out of the flow channel.

The base calling system 2200 may include various components, assemblies, and systems (or sub-systems) that interact with each other to perform a predetermined method or assay protocol for biological or chemical analysis. For example, the base calling system 2200 includes a system controller 2204 that may communicate with the various components, assemblies, and sub-systems of the base calling system 2200 and also the biosensor 2202. For example, in addition to the system receptacle 2212, the base calling system 2200 may also include a fluidic control system 2206 to control the flow of fluid throughout a fluid network of the base calling system 2200 and the biosensor 2202; a fluidic storage system 2208 that is configured to hold all fluids (e.g., gas or liquids) that may be used by the bioassay system; a temperature control system 2210 that may regulate the temperature of the fluid in the fluid network, the fluidic storage system 2208, and/or the biosensor 2202; and an illumination system 2209 that is configured to illuminate the biosensor 2202. As described above, if a cartridge having the biosensor 2202 is loaded into the system receptacle 2212, the cartridge may also include fluidic control and fluidic storage components.

Also shown, the base calling system 2200 may include a user interface 2214 that interacts with the user. For example, the user interface 2214 may include a display 2213 to display or request information from a user and a user input device 2215 to receive user inputs. In some implementations, the display 2213 and the user input device 2215 are the same device. For example, the user interface 2214 may include a touch-sensitive display configured to detect the presence of an individual's touch and also identify a location of the touch on the display. However, other user input devices 2215 may be used, such as a mouse, touchpad, keyboard, keypad, handheld scanner, voice-recognition system, motion-recognition system, and the like. As will be discussed in greater detail below, the base calling system 2200 may communicate with various components, including the biosensor 2202 (e.g., in the form of a cartridge), to perform the desired reactions. The base calling system 2200 may also be configured to analyze data obtained from the biosensor to provide a user with desired information.

The system controller 2204 may include any processor-based or microprocessor-based system, including systems using microcontrollers, Reduced Instruction Set Computers (RISC), Application Specific Integrated Circuits (ASICs), Field Programmable Gate Array (FPGAs), logic circuits, and any other circuit or processor capable of executing functions described herein. The above examples are exemplary only, and are thus not intended to limit in any way the definition and/or meaning of the term system controller. In the exemplary implementation, the system controller 2204 executes a set of instructions that are stored in one or more storage elements, memories, or modules in order to at least one of obtain and analyze detection data. Detection data can include a plurality of sequences of pixel signals, such that a sequence of pixel signals from each of the millions of sensors (or pixels) can be detected over many base calling cycles. Storage elements may be in the form of information sources or physical memory elements within the base calling system 2200.

The set of instructions may include various commands that instruct the base calling system 2200 or biosensor 2202 to perform specific operations such as the methods and processes of the various implementations described herein. The set of instructions may be in the form of a software program, which may form part of a tangible, non-transitory computer readable medium or media. As used herein, the terms “software” and “firmware” are interchangeable and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

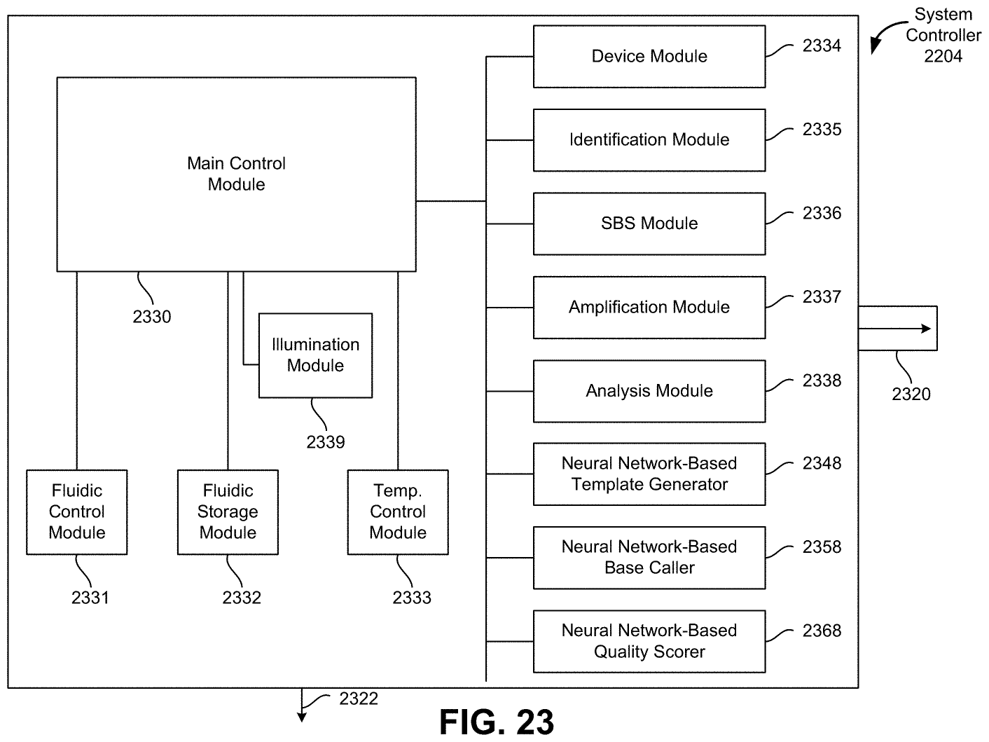

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the base calling system 2200, processed in response to user inputs, or processed in response to a request made by another processing machine (e.g., a remote request through a communication link). In the illustrated implementation, the system controller 2204 includes an analysis module 2338 (illustrated in FIG. 23). In other implementations, system controller 2204 does not include the analysis module 2338 and instead has access to the analysis module 2338 (e.g., the analysis module 2338 may be separately hosted on cloud).

The system controller 2204 may be connected to the biosensor 2202 and the other components of the base calling system 2200 via communication links The system controller 2204 may also be communicatively connected to off-site systems or servers. The communication links may be hardwired, corded, or wireless. The system controller 2204 may receive user inputs or commands, from the user interface 2214 and the user input device 2215.

The fluidic control system 2206 includes a fluid network and is configured to direct and regulate the flow of one or more fluids through the fluid network. The fluid network may be in fluid communication with the biosensor 2202 and the fluidic storage system 2208. For example, select fluids may be drawn from the fluidic storage system 2208 and directed to the biosensor 2202 in a controlled manner, or the fluids may be drawn from the biosensor 2202 and directed toward, for example, a waste reservoir in the fluidic storage system 2208. Although not shown, the fluidic control system 2206 may include flow sensors that detect a flow rate or pressure of the fluids within the fluid network. The sensors may communicate with the system controller 2204.

The temperature control system 2210 is configured to regulate the temperature of fluids at different regions of the fluid network, the fluidic storage system 2208, and/or the biosensor 2202. For example, the temperature control system 2210 may include a thermocycler that interfaces with the biosensor 2202 and controls the temperature of the fluid that flows along the reaction sites in the biosensor 2202. The temperature control system 2210 may also regulate the temperature of solid elements or components of the base calling system 2200 or the biosensor 2202. Although not shown, the temperature control system 2210 may include sensors to detect the temperature of the fluid or other components. The sensors may communicate with the system controller 2204.

The fluidic storage system 2208 is in fluid communication with the biosensor 2202 and may store various reaction components or reactants that are used to conduct the desired reactions therein. The fluidic storage system 2208 may also store fluids for washing or cleaning the fluid network and biosensor 2202 and for diluting the reactants. For example, the fluid storage system 2208 may include various reservoirs to store samples, reagents, enzymes, other biomolecules, buffer solutions, aqueous, and non-polar solutions, and the like. Furthermore, the fluidic storage system 2208 may also include waste reservoirs for receiving waste products from the biosensor 2202. In implementations that include a cartridge, the cartridge may include one or more of a fluid storage system, fluidic control system or temperature control system. Accordingly, one or more of the components set forth herein as relating to those systems can be contained within a cartridge housing. For example, a cartridge can have various reservoirs to store samples, reagents, enzymes, other biomolecules, buffer solutions, aqueous, and non-polar solutions, waste, and the like. As such, one or more of a fluid storage system, fluidic control system or temperature control system can be removably engaged with a bioassay system via a cartridge or other biosensor.

The illumination system 2209 may include a light source (e.g., one or more LEDs) and a plurality of optical components to illuminate the biosensor. Examples of light sources may include lasers, arc lamps, LEDs, or laser diodes. The optical components may be, for example, reflectors, dichroics, beam splitters, collimators, lenses, filters, wedges, prisms, mirrors, detectors, and the like. In implementations that use an illumination system, the illumination system 2209 may be configured to direct an excitation light to reaction sites. As one example, fluorophores may be excited by green wavelengths of light, as such the wavelength of the excitation light may be approximately 532 nm. In one implementation, the illumination system 2209 is configured to produce illumination that is parallel to a surface normal of a surface of the biosensor 2202. In another implementation, the illumination system 2209 is configured to produce illumination that is off-angle relative to the surface normal of the surface of the biosensor 2202. In yet another implementation, the illumination system 2209 is configured to produce illumination that has plural angles, including some parallel illumination and some off-angle illumination

The system receptacle or interface 2212 is configured to engage the biosensor 2202 in at least one of a mechanical, electrical, and fluidic manner. The system receptacle 2212 may hold the biosensor 2202 in a desired orientation to facilitate the flow of fluid through the biosensor 2202. The system receptacle 2212 may also include electrical contacts that are configured to engage the biosensor 2202 so that the base calling system 2200 may communicate with the biosensor 2202 and/or provide power to the biosensor 2202. Furthermore, the system receptacle 2212 may include fluidic ports (e.g., nozzles) that are configured to engage the biosensor 2202. In some implementations, the biosensor 2202 is removably coupled to the system receptacle 2212 in a mechanical manner, in an electrical manner, and also in a fluidic manner.

In addition, the base calling system 2200 may communicate remotely with other systems or networks or with other bioassay systems 2200. Detection data obtained by the bioassay system(s) 2200 may be stored in a remote database.

FIG. 23 is a block diagram of the system controller 2204 that can be used in the system of FIG. 22. In one implementation, the system controller 2204 includes one or more processors or modules that can communicate with one another. Each of the processors or modules may include an algorithm (e.g., instructions stored on a tangible and/or non-transitory computer readable storage medium) or sub-algorithms to perform particular processes. The system controller 2204 is illustrated conceptually as a collection of modules, but may be implemented utilizing any combination of dedicated hardware boards, DSPs, processors, etc. Alternatively, the system controller 2204 may be implemented utilizing an off-the-shelf PC with a single processor or multiple processors, with the functional operations distributed between the processors. As a further option, the modules described below may be implemented utilizing a hybrid configuration in which certain modular functions are performed utilizing dedicated hardware, while the remaining modular functions are performed utilizing an off-the-shelf PC and the like. The modules also may be implemented as software modules within a processing unit.

During operation, a communication port 2320 may transmit information (e.g., commands) to or receive information (e.g., data) from the biosensor 2202 (FIG. 22) and/or the sub-systems 2206, 2208, 2210 (FIG. 22). In implementations, the communication port 2320 may output a plurality of sequences of pixel signals. A communication port 2320 may receive user input from the user interface 2214 (FIG. 22) and transmit data or information to the user interface 2214. Data from the biosensor 2202 or sub-systems 2206, 2208, 2210 may be processed by the system controller 2204 in real-time during a bioassay session. Additionally, or alternatively, data may be stored temporarily in a system memory during a bioassay session and processed in slower than real-time or off-line operation.

As shown in FIG. 23, the system controller 2204 may include a plurality of modules 2331-2339 that communicate with a main control module 2330. The main control module 2330 may communicate with the user interface 2214 (FIG. 22). Although the modules 2331-2339 are shown as communicating directly with the main control module 2330, the modules 2331-2339 may also communicate directly with each other, the user interface 2214, and the biosensor 2202. Also, the modules 2331-2339 may communicate with the main control module 2330 through the other modules.

The plurality of modules 2331-2339 include system modules 2331-2333, 2339 that communicate with the sub-systems 2206, 2208, 2210, and 2209, respectively. The fluidic control module 2331 may communicate with the fluidic control system 2206 to control the valves and flow sensors of the fluid network for controlling the flow of one or more fluids through the fluid network. The fluidic storage module 2332 may notify the user when fluids are low or when the waste reservoir is at or near capacity. The fluidic storage module 2332 may also communicate with the temperature control module 2333 so that the fluids may be stored at a desired temperature. The illumination module 2339 may communicate with the illumination system 2209 to illuminate the reaction sites at designated times during a protocol, such as after the desired reactions (e.g., binding events) have occurred. In some implementations, the illumination module 2339 may communicate with the illumination system 2209 to illuminate the reaction sites at designated angles.

The plurality of modules 2331-2339 may also include a device module 2334 that communicates with the biosensor 2202 and an identification module 2335 that determines identification information relating to the biosensor 2202. The device module 2334 may, for example, communicate with the system receptacle 2212 to confirm that the biosensor has established an electrical and fluidic connection with the base calling system 2200. The identification module 2335 may receive signals that identify the biosensor 2202. The identification module 2335 may use the identity of the biosensor 2202 to provide other information to the user. For example, the identification module 2335 may determine and then display a lot number, a date of manufacture, or a protocol that is recommended to be run with the biosensor 2202.

The plurality of modules 2331-2339 also includes an analysis module 2338 (also called signal processing module or signal processor) that receives and analyzes the signal data (e.g., image data) from the biosensor 2202. Analysis module 2338 includes memory (e.g., RAM or Flash) to store detection data. Detection data can include a plurality of sequences of pixel signals, such that a sequence of pixel signals from each of the millions of sensors (or pixels) can be detected over many base calling cycles. The signal data may be stored for subsequent analysis or may be transmitted to the user interface 2214 to display desired information to the user. In some implementations, the signal data may be processed by the solid-state imager (e.g., CMOS image sensor) before the analysis module 2338 receives the signal data.

The analysis module 2338 is configured to obtain image data from the light detectors at each of a plurality of sequencing cycles. The image data is derived from the emission signals detected by the light detectors and process the image data for each of the plurality of sequencing cycles through a neural network (e.g., a neural network-based template generator 2348, a neural network-based base caller 2358 (e.g., see FIGS. 7, 9, and 10), and/or a neural network-based quality scorer 2368) and produce a base call for at least some of the analytes at each of the plurality of sequencing cycle.

Protocol modules 2336 and 2337 communicate with the main control module 2330 to control the operation of the sub-systems 2206, 2208, and 2210 when conducting predetermined assay protocols. The protocol modules 2336 and 2337 may include sets of instructions for instructing the base calling system 2200 to perform specific operations pursuant to predetermined protocols. As shown, the protocol module may be a sequencing-by-synthesis (SBS) module 2336 that is configured to issue various commands for performing sequencing-by-synthesis processes. In SBS, extension of a nucleic acid primer along a nucleic acid template is monitored to determine the sequence of nucleotides in the template. The underlying chemical process can be polymerization (e.g., as catalyzed by a polymerase enzyme) or ligation (e.g., catalyzed by a ligase enzyme). In a particular polymerase-based SBS implementation, fluorescently labeled nucleotides are added to a primer (thereby extending the primer) in a template dependent fashion such that detection of the order and type of nucleotides added to the primer can be used to determine the sequence of the template. For example, to initiate a first SBS cycle, commands can be given to deliver one or more labeled nucleotides, DNA polymerase, etc., into/through a flow cell that houses an array of nucleic acid templates. The nucleic acid templates may be located at corresponding reaction sites. Those reaction sites where primer extension causes a labeled nucleotide to be incorporated can be detected through an imaging event. During an imaging event, the illumination system 2209 may provide an excitation light to the reaction sites. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for implementations that use reversible termination a command can be given to deliver a deblocking reagent to the flow cell (before or after detection occurs). One or more commands can be given to effect wash(es) between the various delivery steps. The cycle can then be repeated n times to extend the primer by n nucleotides, thereby detecting a sequence of length n. Exemplary sequencing techniques are described, for example, in Bentley et al., Nature 456:53-59 (2008); WO 04/018497; U.S. Pat. No. 7,057,026; WO 91/06678; WO 07/123744; U.S. Pat. Nos. 7,329,492; 7,211,414; 7,315,019; and 7,405,281, each of which is incorporated herein by reference.

For the nucleotide delivery step of an SBS cycle, either a single type of nucleotide can be delivered at a time, or multiple different nucleotide types (e.g., A, C, T and G together) can be delivered. For a nucleotide delivery configuration where only a single type of nucleotide is present at a time, the different nucleotides need not have distinct labels since they can be distinguished based on temporal separation inherent in the individualized delivery. Accordingly, a sequencing method or apparatus can use single color detection. For example, an excitation source need only provide excitation at a single wavelength or in a single range of wavelengths. For a nucleotide delivery configuration where delivery results in multiple different nucleotides being present in the flow cell at one time, sites that incorporate different nucleotide types can be distinguished based on different fluorescent labels that are attached to respective nucleotide types in the mixture. For example, four different nucleotides can be used, each having one of four different fluorophores. In one implementation, the four different fluorophores can be distinguished using excitation in four different regions of the spectrum. For example, four different excitation radiation sources can be used. Alternatively, fewer than four different excitation sources can be used, but optical filtration of the excitation radiation from a single source can be used to produce different ranges of excitation radiation at the flow cell.

In some implementations, fewer than four different colors can be detected in a mixture having four different nucleotides. For example, pairs of nucleotides can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. Exemplary apparatus and methods for distinguishing four different nucleotides using detection of fewer than four colors are described for example in U.S. Pat. App. Ser. Nos. 61/538,294 and 61/619,878, which are incorporated herein by reference in their entireties. U.S. application Ser. No. 13/624,200, which was filed on Sep. 21, 2012, is also incorporated by reference in its entirety.

The plurality of protocol modules may also include a sample-preparation (or generation) module 2337 that is configured to issue commands to the fluidic control system 2206 and the temperature control system 2210 for amplifying a product within the biosensor 2202. For example, the biosensor 2202 may be engaged to the base calling system 2200. The amplification module 2337 may issue instructions to the fluidic control system 2206 to deliver necessary amplification components to reaction chambers within the biosensor 2202. In other implementations, the reaction sites may already contain some components for amplification, such as the template DNA and/or primers. After delivering the amplification components to the reaction chambers, the amplification module 2337 may instruct the temperature control system 2210 to cycle through different temperature stages according to known amplification protocols. In some implementations, the amplification and/or nucleotide incorporation is performed isothermally.

The SBS module 2336 may issue commands to perform bridge PCR where clusters of clonal amplicons are formed on localized areas within a channel of a flow cell. After generating the amplicons through bridge PCR, the amplicons may be “linearized” to make single stranded template DNA, or sstDNA, and a sequencing primer may be hybridized to a universal sequence that flanks a region of interest. For example, a reversible terminator-based sequencing by synthesis method can be used as set forth above or as follows.

Each base calling or sequencing cycle can extend a sstDNA by a single base which can be accomplished for example by using a modified DNA polymerase and a mixture of four types of nucleotides. The different types of nucleotides can have unique fluorescent labels, and each nucleotide can further have a reversible terminator that allows only a single-base incorporation to occur in each cycle. After a single base is added to the sstDNA, excitation light may be incident upon the reaction sites and fluorescent emissions may be detected. After detection, the fluorescent label and the terminator may be chemically cleaved from the sstDNA. Another similar base calling or sequencing cycle may follow. In such a sequencing protocol, the SBS module 2336 may instruct the fluidic control system 2206 to direct a flow of reagent and enzyme solutions through the biosensor 2202. Exemplary reversible terminator-based SBS methods which can be utilized with the apparatus and methods set forth herein are described in US Patent Application Publication No. 2007/0166705 A1, US Patent Application Publication No. 2006/0188901 A1, U.S. Pat. No. 7,057,026, US Patent Application Publication No. 2006/0240439 A1, US Patent Application Publication No. 2006/02814714709 A1, PCT Publication No. WO 05/065814, PCT Publication No. WO 06/064199, each of which is incorporated herein by reference in its entirety. Exemplary reagents for reversible terminator-based SBS are described in U.S. Pat. Nos. 7,541,444; 7,057,026; 7,427,673; 7,566,537; and 7,592,435, each of which is incorporated herein by reference in its entirety.

In some implementations, the amplification and SBS modules may operate in a single assay protocol where, for example, template nucleic acid is amplified and subsequently sequenced within the same cartridge.

The base calling system 2200 may also allow the user to reconfigure an assay protocol. For example, the base calling system 2200 may offer options to the user through the user interface 2214 for modifying the determined protocol. For example, if it is determined that the biosensor 2202 is to be used for amplification, the base calling system 2200 may request a temperature for the annealing cycle. Furthermore, the base calling system 2200 may issue warnings to a user if a user has provided user inputs that are generally not acceptable for the selected assay protocol.

In implementations, the biosensor 2202 includes millions of sensors (or pixels), each of which generates a plurality of sequences of pixel signals over successive base calling cycles. The analysis module 2338 detects the plurality of sequences of pixel signals and attributes them to corresponding sensors (or pixels) in accordance to the row-wise and/or column-wise location of the sensors on an array of sensors.

Each sensor in the array of sensors can produce sensor data for a tile of the flow cell, where a tile in an area on the flow cell at which clusters of genetic material are disposed during the based calling operation. The sensor data can comprise image data in an array of pixels. For a given cycle, the sensor data can include more than one image, producing multiple features per pixel as the tile data.

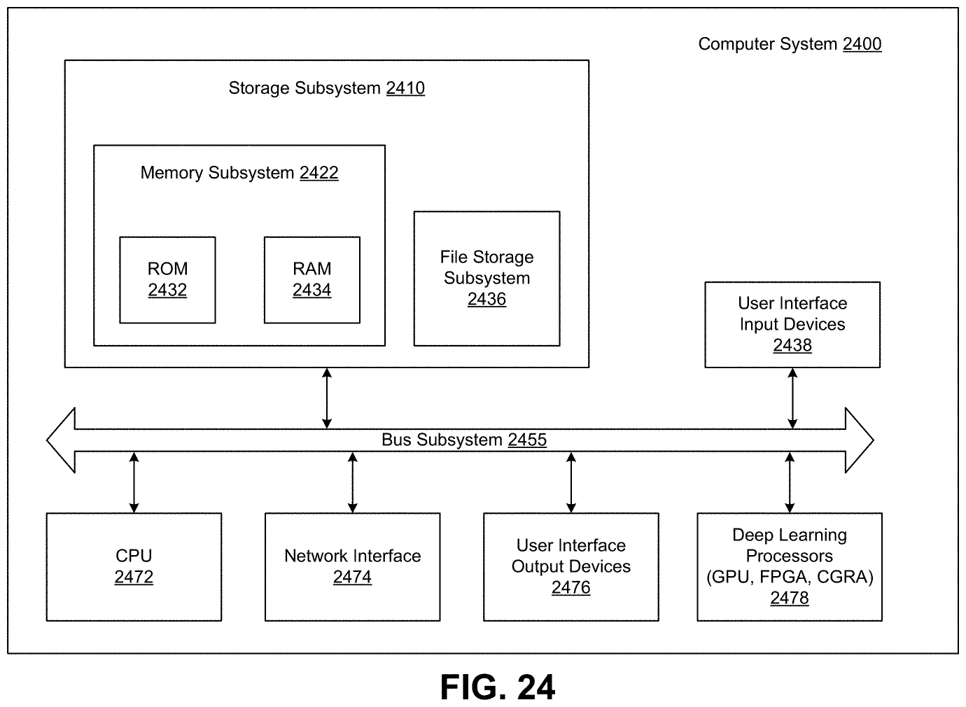

FIG. 24 is a simplified block diagram of a computer 2400 system that can be used to implement the technology disclosed. Computer system 2400 includes at least one central processing unit (CPU) 2472 that communicates with a number of peripheral devices via bus subsystem 2455. These peripheral devices can include a storage subsystem 2410 including, for example, memory devices and a file storage subsystem 2436, user interface input devices 2438, user interface output devices 2476, and a network interface subsystem 2474. The input and output devices allow user interaction with computer system 2400. Network interface subsystem 2474 provides an interface to outside networks, including an interface to corresponding interface devices in other computer systems.

User interface input devices 2438 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2400.

User interface output devices 2476 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2400 to the user or to another machine or computer system.

Storage subsystem 2410 stores programming and data constructs that provide the functionality of some or all of the modules and methods described herein. These software modules are generally executed by deep learning processors 2478.

In one implementation, the neural networks are implemented using deep learning processors 2478 can be configurable and reconfigurable processors, field-programmable gate arrays (FPGAs), application-specific integrated circuits (ASICs), and/or coarse-grained reconfigurable architectures (CGRAs) and graphics processing units (GPUs) other configured devices. Deep learning processors 2478 can be hosted by a deep learning cloud platform such as Google Cloud Platform™, Xilinx™, and Cirrascale™. Examples of deep learning processors 14978 include Google's Tensor Processing Unit (TPU)™, rackmount solutions like GX4 Rackmount Series™, GX149 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM'S DynamicIQ™, IBM TrueNorth™, and others.

Memory subsystem 2422 used in the storage subsystem 2410 can include a number of memories including a main random access memory (RAM) 2434 for storage of instructions and data during program execution and a read only memory (ROM) 2432 in which fixed instructions are stored. A file storage subsystem 2436 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 2436 in the storage subsystem 2410, or in other machines accessible by the processor.

Bus subsystem 2455 provides a mechanism for letting the various components and subsystems of computer system 2400 communicate with each other as intended. Although bus subsystem 2455 is shown schematically as a single bus, alternative implementations of the bus subsystem can use multiple busses.

Computer system 2400 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2400 depicted in FIG. 23 is intended only as a specific example for purposes of illustrating the preferred implementations of the present invention. Many other configurations of computer system 2400 are possible having more or less components than the computer system depicted in FIG. 23.

### CLAUSES

Clause Set 1 (Generation of Final Classification from Classification Information of Two Base Callers)
1. A computer implemented method for base calling using at least two base callers, the method comprising:
executing at least a first base caller and a second base caller on sensor data generated for sensing cycles in a series of sensing cycles;
generating, by the first base caller, first classification information associated with the sensor data, based on executing the first base caller on the sensor data;
generating, by the second base caller, second classification information associated with the sensor data, based on executing the second base caller on the sensor data; and
based on the first classification information and the second classification information, generating a final classification information, the final classification information including one or more base calls for the sensor data.
2. The method of clause 1, wherein at least one of the first base caller and the second base caller implements a non-linear function, and wherein at least another of the first base caller and the second base caller is at least in part linear.
3. The method of clause 1, wherein at least one of the first base caller and the second base caller implements a neural network model, and at least another of the first base caller and the second base caller does not include a neural network model.
4. The method of clause 1, wherein:
the first classification information generated by the first base caller comprises, for each base calling cycle, (i) a first plurality of scores, each score of the first plurality of scores indicative of a probability of the base to be called being one of A, C, T, or G, and (ii) a first called base; and
the second classification information generated by the second base caller comprises, for each base calling cycle, (i) a second plurality of scores, each score of the second plurality of scores indicative of a probability of the base to be called being one of A, C, T, or G, and (ii) a second called base.
5. The method of clause 4, wherein:
the final classification information comprises, for each base calling cycle, (i) a third plurality of scores, each score of the third plurality of scores indicative of a probability of the base to be called being one of A, C, T, or G, and (ii) a final called base.
6. The method of clause 4, wherein at least one of the first base caller and the second base caller uses a softmax function to generate the corresponding plurality of scores.
7. The method of clause 1, wherein generating the final classification information comprises:

generating the final classification information, by selectively combining the first classification information and the second classification information, based on context information associated with the sensor data.
8. The method of clause 7, wherein the context information associated with the sensor data comprises temporal context information, spatial context information, base sequence context information, and other context information.
9. The method of clause 7, wherein the context information associated with the sensor data comprises temporal context information that is indicative of one or more base calling cycle numbers associated with the sensor data.
10. The method of clause 7, wherein the context information associated with the sensor data comprises spatial context information that is indicative of location of one or more tiles within the flow cell that generate the sensor data.
11. The method of clause 7, wherein the context information associated with the sensor data comprises spatial context information that is indicative of location of one or more clusters within a tile of the flow cell that generates the sensor data.
11A. The method of clause 11, wherein the spatial context information is indicative of whether the one or more clusters within the tile of the flow cell, which generates the sensor data, are edge clusters or non-edge clusters.
11B. The method of clause 11A, wherein a cluster is classified as an edge cluster if the cluster is estimated to be located within a threshold distance from an edge of the tile.
11C. The method of clause 11A, wherein a cluster is classified as a non-edge cluster if the cluster is estimated to be located more than a threshold distance from any edge of the tile.
12. The method of clause 7, wherein the context information associated with the sensor data comprises base sequence context information that is indicative of a base sequence being called for the sensor data.
13. The method of clause 1, wherein:
for a specific base to be called, the first classification information comprises a first score, a second score, a third score, and a fourth score indicating probabilities of the base to be called is A, C, T, and G, respectively;
for the specific base to be called, the second classification information comprises a fifth score, a sixth score, a seventh score, and an eighth score indicating probabilities of the base to be called is A, C, T, and G, respectively; and
generating the final classification information comprise:
   generating, for the specific base to be called, the final classification information based on the first score, the second score, the third score, the fourth score, the fifth score, the sixth score, the seventh score, and the eighth score.
14. The method of clause 13, wherein:
the final score comprises a first final score that is a function of the first score and the fifth score, the first final score indicating a probability of the base to be called is A;
the final score comprises a second final score that is a function of the second score and the sixth score, the second final score indicating a probability of the base to be called is C;
the final score comprises a third final score that is a function of the third score and the seventh score, the third final score indicating a probability of the base to be called is T; and
the final score comprises a fourth final score that is a function of the fourth score and the eighth score, the fourth final score indicating a probability of the base to be called is G.
15. The method of clause 14, wherein:
the first final score is an average, a normalized weighted average, a minimum, or a maximum of the first score and the fifth score;
the second final score is an average, a normalized weighted average, a minimum, or a maximum of the second score and the sixth score;
the third final score is an average, a normalized weighted average, a minimum, or a maximum of the third score and the seventh score; and
the fourth final score is an average, a normalized weighted average, a minimum, or a maximum of the fourth score and the eighth score.
16. The method of clause 14, wherein:
for a specific base to be called, the first classification information comprises a first called base that is one of A, C, T, and G, which has a corresponding score that is highest among the first score, the second score, the third score, and the fourth score; and
for the specific base to be called, the second classification information comprises a second called base that is one of A, C, T, and G, which has a corresponding score that is highest among the fifth score, the sixth score, the seventh score, and the eights score.
17. The method of clause 1, wherein:
for a specific base to be called, the first classification information comprises a first called base that is one of A, C, T, and G;
for the specific base to be called, the second classification information comprises a second called base that is same as the first called base; and
generating the final classification information comprise:
   generating, for the specific base to be called, the final classification information, such that the final classification information includes a final called base that matches with the first called base and the second called base.
18. The method of clause 1, wherein:
for a specific base to be called, the first classification information comprises a first called base that is one of A, C, T, and G;
for the specific base to be called, the second classification information comprises a second called base that is another of A, C, T, and G, such that the second called base does not match with the first called base; and
generating the final classification information comprise:
   generating, for the specific base to be called, the final classification information, such that the final classification information includes a final called base that one of (i) the first called base, (ii) the second called base, or (iii) is marked as inconclusive.
19. The method of clause 1, wherein:
at least one of the first classification information, the second classification information, or the final classification information indicates called base sequence to have a specific base sequence pattern; and
in response to the indication of the called base sequence to have the specific base sequence pattern, generating the final classification information by placing a first weight on the first classification information and a second weight on the second classification information, wherein the first weight and the second weight are different.
20. The method of clause 19, wherein:
the specific base sequence pattern comprises a homopolymer pattern or a near-homopolymer pattern.
20a. The method of clause 19, wherein:
the specific base sequence pattern comprises a homopolymer pattern or a pattern with flanking homopolymers.
21. The method of clause 19, wherein:
the specific base sequence pattern comprises a plurality of bases, with at least a first and a last base being a G.
21a. The method of clause 19, wherein:
the specific base sequence pattern comprises at least five bases, with at least a first and a last base being a G.
22. The method of clause 19, wherein:
the specific base sequence pattern comprises a plurality of bases, with a majority of the plurality of bases of the specific base sequence pattern being a G.
22a. The method of clause 19, wherein:
the specific base sequence pattern comprises at least five bases, with at least three bases of the specific base sequence pattern being a G.
22A. The method of clause 19, wherein:
the specific base sequence pattern comprises any of GGXGG, GXGGG, GGGXG, GXXGG, GGXXG, where X is any of A, C, T, or G.
22B. The method of clause 19, wherein:
the specific base sequence pattern comprises a plurality of bases, with each of at least a first and a last base associated with an inactive base call.
22B1. The method of clause 19, wherein:
the specific base sequence pattern comprises at least five bases, with each of at least a first and a last base associated with an inactive base call.
22C. The method of clause 19, wherein:
the specific base sequence pattern comprises a plurality of bases, with base calls of each of at least a first and a last base associated with a dark cycle.
22D. The method of clause 19, wherein:
the specific base sequence pattern comprises a plurality of bases, with each of at least a majority of the bases of the specific base sequence pattern associated with an inactive base call.
22E. The method of clause 19, wherein:
the specific base sequence pattern comprises a plurality of bases, with each of at least a majority of the bases of the specific base sequence pattern associated with a dark cycle.
23. The method of clause 19, wherein:
the first weight is lower than the second weight, such that the first classification information is weighted less than the second classification information, while generating the final classification information.
24. The method of clause 23, wherein:
the first base caller implements a neural network model, and the second base caller does not include a neural network model.
25. The method of clause 19, wherein:
the first weight is higher than 90% and the second weight is lower than 10%.
26. The method of clause 1, wherein:
the sensor data comprises (i) first sensor data for first one or more sensing cycles, and (ii) second sensor data for second one or more sensing cycles that occur subsequent to the first one or more sensing cycles;
the final classification information comprises
   (i) first final classification information for the first one or more sensing cycles that are generated by (a) placing first weight on the first classification information associated with the first one or more sensing cycles and (b) second weight on the second classification information associated with the first one or more sensing cycles, and
   (i) second final classification information for the second one or more sensing cycles that are generated by (a) placing third weight on the first classification information associated with the second one or more sensing cycles and (b) fourth weight on the second classification information associated with the second one or more sensing cycles; and
the first, second, third, and fourth weights are different.
27. The method of clause 26, wherein:
the first base caller implements a neural network model, and the second base caller does not include a neural network model;
the first weight is lower than the second weight, such that for the first one or more sensing cycles, the second classification information from the second base caller is emphasized more than the first classification information from the first base caller; and
the third weight is higher than the fourth weight, such that for the second one or more sensing cycles, the first classification information from the first base caller is emphasized more than the second classification information from the second base caller.
28. The method of clause 1, wherein:
the sensor data comprises (i) first sensor data from first one or more clusters of a tile of a flow cell, and (ii) second sensor data from second one or more clusters of the tile of the flow cell; the final classification information comprises:
   (i) first final classification information for the first sensor data from the first one or more clusters, the first final classification information generated by (a) placing first weight on the first classification information from the first one or more clusters and (b) placing second weight on the second classification information from the first one or more clusters, and
   (i) second final classification information for the second sensor data from the second one or more clusters, the second final classification information generated by (a) placing third weight on the first classification information from the second one or more clusters and (b) placing fourth weight on the second classification information from the second one or more clusters; and
the first, second, third, and fourth weights are different.
29. The method of clause 28, wherein:
the first one or more clusters are edge clusters that are disposed within a threshold distance from one or more edges of the tile of the flow cell; and
the second one or more clusters are non-edge clusters that are disposed beyond the threshold distance from the one or more edges of the tile of the flow cell.
30. The method of clause 29, wherein:
the first base caller implements a neural network model, and the second base caller does not include a neural network model; and
the first weight is higher than the second weight, such that for the first one or more edge clusters, the first classification information from the first base caller is emphasized more than the second classification information from the second base caller.
31. The method of clause 30, wherein:
the third weight is lower than, or equal to, the fourth weight, such that for the second one or more non-edge clusters, the first classification information from the first base caller is emphasized less than or equal to the second classification information from the second base caller.
32. The method of clause 1, further comprising:
detecting, from the sensor data, presence of one or more bubbles in at least one cluster of a tile of a flow cell,
wherein generating the final classification information comprises:
   in response to the detection of the one or more bubbles, generating the final classification information by placing a first weight on the first classification information and a second weight on the second classification information, wherein the first weight and the second weight are different.
33. The method of clause 32, wherein:
the first base caller implements a neural network model, and the second base caller does not include a neural network model; and
the first weight is higher than the second weight, such that in response to the detection of the one or more bubbles, the first classification information from the first base caller is emphasized more than the second classification information from the second base caller.
34. The method of clause 1, wherein the sensor data comprises at least one image, and wherein the method further comprises:
detecting that the at least one image is an out of focus image,
wherein generating the final classification information comprises:
   in response to the detection of the out of focus image, generating the final classification information by placing a first weight on the first classification information and a second weight on the second classification information, wherein the first weight and the second weight are different.
35. The method of clause 32, wherein:
the first base caller implements a neural network model, and the second base caller does not include a neural network model; and
the first weight is higher than the second weight, such that in response to the detection of the out of focus image, the first classification information from the first base caller is emphasized more than the second classification information from the second base caller.
36. The method of clause 1, wherein:
the sensor data is associated with a plurality of sequencing cycles;
the first classification information includes a first called base sequence corresponding to the plurality of sequencing cycles, and the second classification information includes a second called base sequence corresponding to the plurality of sequencing cycles;
the first called base sequence and the second called base sequence does not match, and at least one of the first or second called base sequences has a specific base sequence pattern;
the first base caller implements a neural network model, and the second base caller does not include a neural network model; and
generating the final classification information comprises:
   in response to (i) at least one of the first or second called base sequences having the specific base sequence pattern and (ii) the second base caller not including the neural network model, generating the final classification information such that a final called base sequence of the final classification information matches with second called base sequence and does not match with the first called base sequence.
37. The method of clause 36, wherein:
the specific base sequence pattern comprises a homopolymer pattern or a near-homopolymer pattern.
38. The method of clause 36, wherein:
the specific base sequence pattern comprises a plurality of bases, with at least a first and a last base being a G.
39. The method of clause 36, wherein:
the specific base sequence pattern comprises a plurality of bases, with at least a majority of the bases of the specific base sequence pattern being a G.
39a. The method of clause 36, wherein:
the specific base sequence pattern comprises at least five bases, with at least three bases of the specific base sequence pattern being a G.
39A. The method of clause 36, wherein:
the specific base sequence pattern comprises a plurality of bases, with each of at least a first and a last base associated with an inactive base call.
39B. The method of clause 36, wherein:
the specific base sequence pattern comprises a plurality of bases, with base calls of each of at least a first and a last base associated with a dark cycle.
39C. The method of clause 36, wherein:
the specific base sequence pattern comprises a plurality of bases, with each of at least a majority of the bases of the specific base sequence pattern associated with an inactive base call.
39D. The method of clause 36, wherein:
the specific base sequence pattern comprises a plurality of bases, with each of at least a majority of the bases of the specific base sequence pattern associated with a dark cycle.
40. The method of clause 1, wherein generating the final classification information comprises:
receiving, by a machine learning model, the first classification information associated with the sensor data, from the first base caller;
receiving, by the machine learning model, the second classification information associated with the sensor data, from the second base caller; and
generating, by the machine learning model, the final classification information, based on the first classification information and the second classification information.
40a. The method of clause 40, wherein the machine learning model is any of a logistic regression model, a gradient boosted trees model, a random forest model, Naïve Bayes model, or a neural network model.
40b. The method of clause 1, wherein generating the final classification information comprises:
receiving, by a neural network model, the first classification information associated with the sensor data, from the first base caller;
receiving, by the neural network model, the second classification information associated with the sensor data, from the second base caller; and
generating, by the neural network model, the final classification information, based on the first classification information and the second classification information.
41. A computer implemented method comprising:
generating sensor data for sensing cycles in the series of sensing cycles; and
executing at least a first base caller and a second base caller on at least corresponding portions of the sensor data, and selectively switching execution of the first and second base callers, based on context information associated with the sensor data, wherein the first base caller is different from the second base caller;
generating, by the first base caller and the second base caller, first classification information and second classification information, respectively,
generating base calls, based on one or both of the first classification information and the second classification information.
42. A non-transitory computer readable storage medium impressed with computer program instructions to progressively train a base caller, the instructions, when executed on a processor, implement a method comprising:
executing at least a first base caller and a second base caller on sensor data generated for sensing cycles in a series of sensing cycles;
generating, by the first base caller, first classification information associated with the sensor data, based on executing the first base caller on the sensor data;
generating, by the second base caller, second classification information associated with the sensor data, based on executing the second base caller on the sensor data; and
based on the first classification information and the second classification information, generating a final classification information, the final classification information including one or more base calls for the sensor data.
43. The non-transitory computer readable storage medium of clause 42, wherein at least one of the first base caller and the second base caller implements a non-linear function, and wherein at least another of the first base caller and the second base caller is at least in part linear.
44. The non-transitory computer readable storage medium of clause 42, wherein at least one of the first base caller and the second base caller implements a neural network model, and at least another of the first base caller and the second base caller does not include a neural network model.
45. The non-transitory computer readable storage medium of clause 42, wherein:
the first classification information generated by the first base caller comprises, for each base calling cycle, (i) a first plurality of scores, each score of the first plurality of scores indicative of a probability of the base to be called being one of A, C, T, or G, and (ii) a first called base; and
the second classification information generated by the second base caller comprises, for each base calling cycle, (i) a second plurality of scores, each score of the second plurality of scores indicative of a probability of the base to be called being one of A, C, T, or G, and (ii) a second called base.
46. The non-transitory computer readable storage medium of clause 45, wherein:
the final classification information comprises, for each base calling cycle, (i) a third plurality of scores, each score of the third plurality of scores indicative of a probability of the base to be called being one of A, C, T, or G, and (ii) a final called base.
47. The non-transitory computer readable storage medium of clause 45, wherein at least one of the first base caller and the second base caller uses a softmax function to generate the corresponding plurality of scores.
48. The non-transitory computer readable storage medium of clause 42, wherein generating the final classification information comprises:
generating the final classification information, by selectively combining the first classification information and the second classification information, based on context information associated with the sensor data.
49. The non-transitory computer readable storage medium of clause 48, wherein the context information associated with the sensor data comprises temporal context information, spatial context information, base sequence context information, and other context information.
50. The non-transitory computer readable storage medium of clause 48, wherein the context information associated with the sensor data comprises temporal context information that is indicative of one or more base calling cycle numbers associated with the sensor data.
51. The non-transitory computer readable storage medium of clause 48, wherein the context information associated with the sensor data comprises spatial context information that is indicative of location of one or more tiles within the flow cell that generate the sensor data.
52. The non-transitory computer readable storage medium of clause 48, wherein the context information associated with the sensor data comprises spatial context information that is indicative of location of one or more clusters within a tile of the flow cell that generates the sensor data.
52A. The non-transitory computer readable storage medium of clause 52, wherein the spatial context information is indicative of whether the one or more clusters within the tile of the flow cell, which generates the sensor data, are edge clusters or non-edge clusters.
52B. The non-transitory computer readable storage medium of clause 52A, wherein a cluster is classified as an edge cluster if the cluster is estimated to be located within a threshold distance from an edge of the tile.
52C. The non-transitory computer readable storage medium of clause 52A, wherein a cluster is classified as a non-edge cluster if the cluster is estimated to be located more than a threshold distance from any edge of the tile.
53. The non-transitory computer readable storage medium of clause 48, wherein the context information associated with the sensor data comprises base sequence context information that is indicative of a base sequence being called for the sensor data.
54. The non-transitory computer readable storage medium of clause 42, wherein:
for a specific base to be called, the first classification information comprises a first score, a second score, a third score, and a fourth score indicating probabilities of the base to be called is A, C, T, and G, respectively;
for the specific base to be called, the second classification information comprises a fifth score, a sixth score, a seventh score, and an eighth score indicating probabilities of the base to be called is A, C, T, and G, respectively; and
generating the final classification information comprise:
generating, for the specific base to be called, the final classification information based on the first score, the second score, the third score, the fourth score, the fifth score, the sixth score, the seventh score, and the eighth score.
55. The non-transitory computer readable storage medium of clause 54, wherein:
the final score comprises a first final score that is a function of the first score and the fifth score, the first final score indicating a probability of the base to be called is A;
the final score comprises a second final score that is a function of the second score and the sixth score, the second final score indicating a probability of the base to be called is C;
the final score comprises a third final score that is a function of the third score and the seventh score, the third final score indicating a probability of the base to be called is T; and
the final score comprises a fourth final score that is a function of the fourth score and the eighth score, the fourth final score indicating a probability of the base to be called is G.
56. The non-transitory computer readable storage medium of clause 55, wherein:
the first final score is an average, a normalized weighted average, a minimum, or a maximum of the first score and the fifth score;
   the second final score is an average, a normalized weighted average, a minimum, or a maximum of the second score and the sixth score;
   the third final score is an average, a normalized weighted average, a minimum, or a maximum of the third score and the seventh score; and
   the fourth final score is an average, a normalized weighted average, a minimum, or a maximum of the fourth score and the eighth score.
57. The non-transitory computer readable storage medium of clause 55, wherein:
for a specific base to be called, the first classification information comprises a first called base that is one of A, C, T, and G, which has a corresponding score that is highest among the first score, the second score, the third score, and the fourth score; and
for the specific base to be called, the second classification information comprises a second called base that is one of A, C, T, and G, which has a corresponding score that is highest among the fifth score, the sixth score, the seventh score, and the eights score.
58. The non-transitory computer readable storage medium of clause 42, wherein:
for a specific base to be called, the first classification information comprises a first called base that is one of A, C, T, and G;
   for the specific base to be called, the second classification information comprises a second called base that is same as the first called base; and
   generating the final classification information comprise:
      generating, for the specific base to be called, the final classification information, such that the final classification information includes a final called base that matches with the first called base and the second called base.
59. The non-transitory computer readable storage medium of clause 42, wherein:
for a specific base to be called, the first classification information comprises a first called base that is one of A, C, T, and G;
   for the specific base to be called, the second classification information comprises a second called base that is another of A, C, T, and G, such that the second called base does not match with the first called base; and
   generating the final classification information comprise:
      generating, for the specific base to be called, the final classification information, such that the final classification information includes a final called base that one of (i) the first called base, (ii) the second called base, or (iii) is marked as inconclusive.
60. The non-transitory computer readable storage medium of clause 42, wherein:
at least one of the first classification information, the second classification information, or the final classification information indicates called base sequence to have a specific base sequence pattern; and
   in response to the indication of the called base sequence to have the specific base sequence pattern, generating the final classification information by placing a first weight on the first classification information and a second weight on the second classification information, wherein the first weight and the second weight are different.
61. The non-transitory computer readable storage medium of clause 60, wherein:
the specific base sequence pattern comprises a homopolymer pattern or a near-homopolymer pattern.
62. The non-transitory computer readable storage medium of clause 60, wherein:
the specific base sequence pattern comprises a plurality of bases, with at least a first and a last base being a G.
63. The non-transitory computer readable storage medium of clause 60, wherein:
the specific base sequence pattern comprises a plurality of bases, with a majority of the bases of the specific base sequence pattern being a G.
63A. The non-transitory computer readable storage medium of clause 60, wherein:
the specific base sequence pattern comprises any of GGXGG, GXGGG, GGGXG, GXXGG, GGXXG, where X is any of A, C, T, or G.
63B. The non-transitory computer readable storage medium of clause 60, wherein:
the specific base sequence pattern comprises a plurality of bases, with each of at least a first and a last base associated with an inactive base call.
63C. The non-transitory computer readable storage medium of clause 60, wherein:
the specific base sequence pattern comprises a plurality of bases, with base calls of each of at least a first and a last base associated with a dark cycle.
63D. The non-transitory computer readable storage medium of clause 60, wherein:
the specific base sequence pattern comprises a plurality of bases, with a majority of the bases of the specific base sequence pattern associated with an inactive base call.
63E. The non-transitory computer readable storage medium of clause 60, wherein:
the specific base sequence pattern comprises at least five bases, with each of at least three bases of the specific base sequence pattern associated with a dark cycle.
64. The non-transitory computer readable storage medium of clause 60, wherein:
the first weight is lower than the second weight, such that the first classification information is weighted less than the second classification information, while generating the final classification information.
65. The non-transitory computer readable storage medium of clause 64, wherein:
the first base caller implements a neural network model, and the second base caller does not include a neural network model.
66. The non-transitory computer readable storage medium of clause 60, wherein:
the first weight is higher than 90% and the second weight is lower than 10%.
67. The non-transitory computer readable storage medium of clause 42, wherein:
the sensor data comprises (i) first sensor data for first one or more sensing cycles, and (ii) second sensor data for second one or more sensing cycles that occur subsequent to the first one or more sensing cycles;
the final classification information comprises
   (i) first final classification information for the first one or more sensing cycles that are generated by (a) placing first weight on the first classification information associated with the first one or more sensing cycles and (b) second weight on the second classification information associated with the first one or more sensing cycles, and
   (i) second final classification information for the second one or more sensing cycles that are generated by (a) placing third weight on the first classification information associated with the second one or more sensing cycles and (b) fourth weight on the second classification information associated with the second one or more sensing cycles; and
the first, second, third, and fourth weights are different.
68. The non-transitory computer readable storage medium of clause 67, wherein:
the first base caller implements a neural network model, and the second base caller does not include a neural network model;
the first weight is lower than the second weight, such that for the first one or more sensing cycles, the second classification information from the second base caller is emphasized more than the first classification information from the first base caller; and
the third weight is higher than the fourth weight, such that for the second one or more sensing cycles, the first classification information from the first base caller is emphasized more than the second classification information from the second base caller.
69. The non-transitory computer readable storage medium of clause 42, wherein:
the sensor data comprises (i) first sensor data from first one or more clusters of a tile of a flow cell, and (ii) second sensor data from second one or more clusters of the tile of the flow cell; the final classification information comprises:
   (i) first final classification information for the first sensor data from the first one or more clusters, the first final classification information generated by (a) placing first weight on the first classification information from the first one or more clusters and (b) placing second weight on the second classification information from the first one or more clusters, and
   (i) second final classification information for the second sensor data from the second one or more clusters, the second final classification information generated by (a) placing third weight on the first classification information from the second one or more clusters and (b) placing fourth weight on the second classification information from the second one or more clusters; and
the first, second, third, and fourth weights are different.
70. The non-transitory computer readable storage medium of clause 69, wherein:
the first one or more clusters are edge clusters that are disposed within a threshold distance from one or more edges of the tile of the flow cell; and
the second one or more clusters are non-edge clusters that are disposed beyond the threshold distance from the one or more edges of the tile of the flow cell.
71. The non-transitory computer readable storage medium of clause 70, wherein:
the first base caller implements a neural network model, and the second base caller does not include a neural network model; and
the first weight is higher than the second weight, such that for the first one or more edge clusters, the first classification information from the first base caller is emphasized more than the second classification information from the second base caller.
72. The non-transitory computer readable storage medium of clause 71, wherein:
the third weight is lower than, or equal to, the fourth weight, such that for the second one or more non-edge clusters, the first classification information from the first base caller is emphasized less than or equal to the second classification information from the second base caller.
73. The non-transitory computer readable storage medium of clause 42, further comprising:
detecting, from the sensor data, presence of one or more bubbles in at least one cluster of a tile of a flow cell,
wherein generating the final classification information comprises:
   in response to the detection of the one or more bubbles, generating the final classification information by placing a first weight on the first classification information and a second weight on the second classification information, wherein the first weight and the second weight are different.
74. The non-transitory computer readable storage medium of clause 73, wherein:
the first base caller implements a neural network model, and the second base caller does not include a neural network model; and
the first weight is higher than the second weight, such that in response to the detection of the one or more bubbles, the first classification information from the first base caller is emphasized more than the second classification information from the second base caller.
75. The non-transitory computer readable storage medium of clause 73, wherein the sensor data comprises at least one image, and wherein the method further comprises:
detecting that the at least one image is an out of focus image,
wherein generating the final classification information comprises:
   in response to the detection of the out of focus image, generating the final classification information by placing a first weight on the first classification information and a second weight on the second classification information, wherein the first weight and the second weight are different.
76. The non-transitory computer readable storage medium of clause 73, wherein:
the first base caller implements a neural network model, and the second base caller does not include a neural network model; and
the first weight is higher than the second weight, such that in response to the detection of the out of focus image, the first classification information from the first base caller is emphasized more than the second classification information from the second base caller.
77. The non-transitory computer readable storage medium of clause 42, wherein:
the sensor data is associated with a plurality of sequencing cycles;
the first classification information includes a first called base sequence corresponding to the plurality of sequencing cycles, and the second classification information includes a second called base sequence corresponding to the plurality of sequencing cycles;
the first called base sequence and the second called base sequence does not match, and at least one of the first or second called base sequences has a specific base sequence pattern;
the first base caller implements a neural network model, and the second base caller does not include a neural network model; and
generating the final classification information comprises:
   in response to (i) at least one of the first or second called base sequences having the specific base sequence pattern and (ii) the second base caller not including the neural network model, generating the final classification information such that a final called base sequence of the final classification information matches with second called base sequence and does not match with the first called base sequence.
78. The non-transitory computer readable storage medium of clause 77, wherein:
the specific base sequence pattern comprises a homopolymer pattern or a near-homopolymer pattern.
79. The non-transitory computer readable storage medium of clause 77, wherein:
the specific base sequence pattern comprises a plurality of bases, with at least a first and a last base being a G.
80. The non-transitory computer readable storage medium of clause 77, wherein:
the specific base sequence pattern comprises a plurality of bases, with a majority of the bases of the specific base sequence pattern being a G.
80A. The non-transitory computer readable storage medium of clause 77, wherein:
the specific base sequence pattern comprises a plurality of bases, with each of at least a first and a last base associated with an inactive base call.
80B. The non-transitory computer readable storage medium of clause 77, wherein:
the specific base sequence pattern comprises a plurality of bases, with base calls of each of at least a first and a last base associated with a dark cycle.
80C. The non-transitory computer readable storage medium of clause 77, wherein:
the specific base sequence pattern comprises a plurality of bases, with a majority of the plurality of bases of the specific base sequence pattern associated with an inactive base call.
80D. The non-transitory computer readable storage medium of clause 60, wherein:
the specific base sequence pattern comprises at least five bases, with each of at least three bases of the specific base sequence pattern associated with a dark cycle.
81. The non-transitory computer readable storage medium of clause 42, wherein generating the final classification information comprises:
receiving, by a neural network model, the first classification information associated with the sensor data, from the first base caller;
receiving, by the neural network model, the second classification information associated with the sensor data, from the second base caller; and
generating, by the neural network model, the final classification information, based on the first classification information and the second classification information.

### Clause Set 2 (Switching/Selectively Enabling the Two Base Callers)

1. A computer implemented method for base calling using at least two base callers, the method comprising:
executing a first base caller on sensor data generated for sensing cycles in a series of sensing cycles;
generating, by the first base caller, first classification information associated with the sensor data, based on the executing the first base caller on the sensor data;
determining that the first classification information is inadequate for generation of final classification information for the sensor data;
in response to determining the inadequacy of the first classification information, executing a second base caller on the sensor data, the second base caller different from the first base caller;
generating, by the second base caller, second classification information associated with the sensor data, based on the executing the second base caller on the sensor data; and
based on the first classification information and the second classification information, generating the final classification information, the final classification information including one or more base calls for the sensor data.
2. The method of clause 1, wherein the first classification information includes first called base sequence, and wherein determining that the first classification information is inadequate comprises:
determining that the first called base sequence matches a specific base sequence pattern; and
based on the first called base sequence matching the specific base sequence pattern, determining that the first classification information is inadequate for generation of the final classification information.
3. The method of clause 2, wherein:
the specific base sequence pattern comprises a homopolymer pattern or a near-homopolymer pattern.
4. The method of clause 2, wherein:
the specific base sequence pattern comprises a plurality of bases, with at least a first and a last base of the plurality of bases being a G.
4A. The method of clause 2, wherein:
the specific base sequence pattern comprises at least five bases, with at least a first and a last base being a G.
5. The method of clause 2, wherein:
the specific base sequence pattern comprises a plurality of bases, with at least three bases of the plurality of bases being a G.
5A. The method of clause 2, wherein:
the specific base sequence pattern comprises at least five bases, with at least three bases of the specific base sequence pattern being a G.
6. The method of clause 2, wherein:
the specific base sequence pattern comprises any of GGXGG, GXGGG, GGGXG, GXXGG, GGXXG, where X is any of A, C, T, or G.
6A. The method of clause 2, wherein:
the specific base sequence pattern comprises a plurality of bases, with each of at least a first and a last base associated with an inactive base call.
6B. The method of clause 2, wherein:
the specific base sequence pattern comprises a plurality of bases, with base calls of each of at least a first and a last base associated with a dark cycle.
6C. The method of clause 2, wherein:
the specific base sequence pattern comprises at least five bases, with each of at least three bases of the specific base sequence pattern associated with an inactive base call.
6D. The method of clause 2, wherein:
the specific base sequence pattern comprises at least five bases, with each of at least three bases of the specific base sequence pattern associated with a dark cycle.
7. The method of clause 2, wherein generating the final classification information comprises:
in response to the first called base sequence matching the specific base sequence pattern, generating the final classification information by placing a first weight on the first classification information and a second weight on the second classification information, wherein the first weight and the second weight are different.
8. The method of clause 7, wherein:
the first base caller implements a neural network model, and the second base caller does not include a neural network model; and
the first weight is lower than the second weight, such that while generating the final classification information, the first classification information is weighted less than the second classification information.
9. The method of clause 2, wherein:
the first base caller implements a neural network model, and the second base caller does not include a neural network model;
the second classification information includes second called base sequence;
the first called base sequence does not match with the second called base sequence; and
in response to (i) the first called base sequence matching the specific base sequence pattern and (ii) the second base caller not including the neural network model, generating the final classification information, such that a final called base sequence of the final classification information matches with second called base sequence and does not match with the first called base sequence.
10. The method of clause 1, wherein determining that the first classification information is inadequate comprises:
detecting presence of a bubble in a cluster from which the sensor data is generated; and
based on the detection of the bubble, determining that the first classification information is inadequate for generation of the final classification information.
11. The method of clause 10, wherein the second base caller implements a neural network model, and the first base caller does not include a neural network model, and generating the final classification information comprises:
generating the final classification information by placing a first weight on the first classification information and a second weight on the second classification information, wherein the second weight is more than the first weight.
12. The method of clause 1, wherein:
the sensor data is current sensor data;
the current sensor data is for sensing cycle N1 and one or more subsequent sensing cycles, where N1 is a positive integer greater than 1; and
executing the second base caller on the current sensor data comprises:
   initially executing the second base caller on past sensor data associated with at least T number of sensing cycles that occur prior to the sensing cycle N1, to estimate phasing data associated with the at least T number of sensing cycles, and
   subsequently executing the second base caller on the current sensor data associated with the sensing cycle N1 and one or more subsequent sensing cycles, using the estimated phasing data.
13. The method of clause 1, wherein the sensor data is first sensor data generated from first one or more clusters of a tile of a flow cell, and wherein the method further comprises:
generating second sensor data from second one or more clusters of the tile of the flow cell for sensing cycles in the series of sensing cycles; and
executing the first base caller on the second sensor data;
generating, by the first base caller, third classification information associated with the second sensor data, based on the executing the first base caller on the second sensor data;
determining that the third classification information is adequate for generation of final classification information for the second one or more clusters;
wherein executing the second base caller on the first sensor data comprises:
   executing the second base caller on the first sensor data, without executing the second base caller on the second sensor data, such that (i) the final classification for the first one or more clusters are based on output of the first and second base callers, and (ii) the final classification for the second one or more clusters are based on output of the first base caller but not the second base caller.
14. The method of clause 1, wherein determining that the first classification information is inadequate comprises:
receiving context information associated with the sensor data;
determining, based on the context data, that the first classification information includes a probability of error that is higher than a threshold probability; and
based on determining that the first classification information includes the probability of error higher than the threshold probability, determining that the first classification information is inadequate for generation of final classification information for the sensor data.
15. A system for base calling, comprising:
memory storing images that depict intensity emissions of a set of analytes, the intensity emissions generated by analytes in the set of analytes during sequencing cycles of a sequencing run, wherein the memory further stores a topology of a first base caller and a second base caller;
a context information generation module configured to generate context information associated with the images; one or more processors configured to execute the first base caller on the images, thereby generating first classification information associated with the images; and
a final base call determination module configured to determine insufficiency of the first classification information in generating final classification information associated with the images,
wherein in response to the determination of the insufficiency of the first classification information, the one or more processors are configured to execute the second base caller on the images, thereby generating second classification information associated with the images, and
wherein the final base call determination module is further configured to generate the final classification information that includes one or more final base calls of the sequencing run, based at least in part on the second classification information.
16. The system of clause 15, wherein the final classification information is generated further based at least in part on the first classification information.
17. The system of clause 15, wherein the final classification information is generated based on a weighted sum of the first classification information and the second classification information.
18. The system of clause 15, wherein the first classification information includes first called base sequence, and wherein to determine the insufficiency of the first classification information, the final base call determination module is to:
determine that the first called base sequence matches a specific base sequence pattern; and
based on the first called base sequence matching the specific base sequence pattern, determining that the first classification information is inadequate for generation of final classification information.
19. The system of clause 18, wherein:
the specific base sequence pattern comprises a homopolymer pattern or a near-homopolymer pattern.
20. The system of clause 18, wherein:
the specific base sequence pattern comprises a plurality of bases, with at least a first and a last base being a G.
21. The system of clause 18, wherein:
the specific base sequence pattern comprises at least five bases, with at least three bases of the specific base sequence pattern being a G.
22. The system of clause 18, wherein:
the specific base sequence pattern comprises any of GGXGG, GXGGG, GGGXG, GXXGG, GGXXG, where X is any of A, C, T, or G.
23A. The system of clause 18, wherein:
the specific base sequence pattern comprises a plurality of bases, with each of at least a first and a last base associated with an inactive base call.
23B. The system of clause 18, wherein:
the specific base sequence pattern comprises a plurality of bases, with base calls of each of at least a first and a last base associated with a dark cycle.
23C. The system of clause 18, wherein:
the specific base sequence pattern comprises at least five bases, with each of at least three bases of the specific base sequence pattern associated with an inactive base call.
23D. The system of clause 18, wherein:
the specific base sequence pattern comprises at least five bases, with each of at least three bases of the specific base sequence pattern associated with a dark cycle.
23. The system of clause 15, wherein to determine the insufficiency of the first classification information, the final base call determination module is to:
detect presence of a bubble in a cluster from which the images are generated; and
based on the detection of the bubble, determine that the first classification information is insufficient for generation of the final classification information.
24. The system of clause 15, wherein to determine the insufficiency of the first classification information, the final base call determination module is to:
detect presence of an out of focus image within the images; and
based on the detection of the out of focus image, determine that the first classification information is insufficient for generation of the final classification information.
25. A non-transitory computer readable storage medium impressed with computer program instructions to progressively train a base caller, the instructions, when executed on a processor, implement a method comprising:
executing a first base caller on sensor data generated for sensing cycles in a series of sensing cycles, to generate first classification information associated with the sensor data;
processing (i) context information associated with the sensor data and (ii) the first classification information; based on processing the context information and the first classification information, executing a second base caller on the sensor data, to generate second classification information associated with the sensor data; and
based on the first classification information and the second classification information, generating the final classification information, the final classification information including one or more base calls for the sensor data.

What is claimed is:

1. A computer implemented method for base calling using at least two base callers, the method comprising:
executing at least a first base caller and a second base caller on sensor data generated for sensing cycles in a series of sensing cycles;
generating, by the first base caller, first classification information associated with the sensor data, based on executing the first base caller on the sensor data;
generating, by the second base caller, second classification information associated with the sensor data, based on executing the second base caller on the sensor data; and
based on the first classification information and the second classification information, generating a final classification information, the final classification information including one or more base calls for the sensor data.

2. The method of claim 1, wherein at least one of the first base caller and the second base caller implements a non-linear function, and wherein at least another of the first base caller and the second base caller is at least in part linear.

3. The method of claim 1, wherein at least one of the first base caller and the second base caller implements a neural network model, and at least another of the first base caller and the second base caller does not include a neural network model.

4. The method of claim 1, wherein:
the first classification information generated by the first base caller comprises, for each base calling cycle, (i) a first plurality of scores, each score of the first plurality of scores indicative of a probability of the base to be called being one of A, C, T, or G, and (ii) a first called base; and
the second classification information generated by the second base caller comprises, for each base calling cycle, (i) a second plurality of scores, each score of the second plurality of scores indicative of a probability of the base to be called being one of A, C, T, or G, and (ii) a second called base.

5. The method of claim 4, wherein:
the final classification information comprises, for each base calling cycle, (i) a third plurality of scores, each score of the third plurality of scores indicative of a probability of the base to be called being one of A, C, T, or G, and (ii) a final called base.

6. The method of claim 4, wherein at least one of the first base caller and the second base caller uses a softmax function to generate the corresponding plurality of scores.

7. The method of claim 1, wherein generating the final classification information comprises:
generating the final classification information, by selectively combining the first classification information and the second classification information, based on context information associated with the sensor data.

8. The method of claim 7, wherein the context information associated with the sensor data comprises temporal context information, spatial context information, base sequence context information, and other context information.

9. The method of claim 7, wherein the context information associated with the sensor data comprises temporal context information that is indicative of one or more base calling cycle numbers associated with the sensor data.

10. The method of claim 7, wherein the context information associated with the sensor data comprises spatial context information that is indicative of location of one or more tiles within the flow cell that generate the sensor data.

11. The method of claim 7, wherein the context information associated with the sensor data comprises spatial context information that is indicative of location of one or more clusters within a tile of the flow cell that generates the sensor data.

12. The method of claim 11, wherein the spatial context information is indicative of whether the one or more clusters within the tile of the flow cell, which generates the sensor data, are edge clusters or non-edge clusters.

13. The method of claim 11, wherein a cluster is classified as an edge cluster if the cluster is estimated to be located within a threshold distance from an edge of the tile.

14. The method of claim 11, wherein a cluster is classified as a non-edge cluster if the cluster is estimated to be located more than a threshold distance from any edge of the tile.

15. The method of claim 7, wherein the context information associated with the sensor data comprises base sequence context information that is indicative of a base sequence being called for the sensor data.

16. The method of claim 1, wherein:
for a specific base to be called, the first classification information comprises a first score, a second score, a third score, and a fourth score indicating probabilities of the base to be called is A, C, T, and G, respectively;
for the specific base to be called, the second classification information comprises a fifth score, a sixth score, a seventh score, and an eighth score indicating probabilities of the base to be called is A, C, T, and G, respectively; and
generating the final classification information comprise:
generating, for the specific base to be called, the final classification information based on the first score, the second score, the third score, the fourth score, the fifth score, the sixth score, the seventh score, and the eighth score.

17. The method of claim 16, wherein:
the final score comprises a first final score that is a function of the first score and the fifth score, the first final score indicating a probability of the base to be called is A;
the final score comprises a second final score that is a function of the second score and the sixth score, the second final score indicating a probability of the base to be called is C;
the final score comprises a third final score that is a function of the third score and the seventh score, the third final score indicating a probability of the base to be called is T; and
the final score comprises a fourth final score that is a function of the fourth score and the eighth score, the fourth final score indicating a probability of the base to be called is G.

18. The method of claim 17, wherein:
the first final score is an average, a normalized weighted average, a minimum, or a maximum of the first score and the fifth score;
the second final score is an average, a normalized weighted average, a minimum, or a maximum of the second score and the sixth score;
the third final score is an average, a normalized weighted average, a minimum, or a maximum of the third score and the seventh score; and
the fourth final score is an average, a normalized weighted average, a minimum, or a maximum of the fourth score and the eighth score.

19. The method of claim 17, wherein:
for a specific base to be called, the first classification information comprises a first called base that is one of A, C, T, and G, which has a corresponding score that is highest among the first score, the second score, the third score, and the fourth score; and
for the specific base to be called, the second classification information comprises a second called base that is one of A, C, T, and G, which has a corresponding score that is highest among the fifth score, the sixth score, the seventh score, and the eights score.

20. The method of claim 1, wherein:
for a specific base to be called, the first classification information comprises a first called base that is one of A, C, T, and G;
for the specific base to be called, the second classification information comprises a second called base that is same as the first called base; and
generating the final classification information comprise:
generating, for the specific base to be called, the final classification information, such that the final classification information includes a final called base that matches with the first called base and the second called base.

21. The method of claim 1, wherein:
for a specific base to be called, the first classification information comprises a first called base that is one of A, C, T, and G;
for the specific base to be called, the second classification information comprises a second called base that is another of A, C, T, and G, such that the second called base does not match with the first called base; and
generating the final classification information comprise:
generating, for the specific base to be called, the final classification information, such that the final classification information includes a final called base that one of (i) the first called base, (ii) the second called base, or (iii) is marked as inconclusive.

22. The method of claim 1, wherein:
at least one of the first classification information, the second classification information, or the final classification information indicates called base sequence to have a specific base sequence pattern; and
in response to the indication of the called base sequence to have the specific base sequence pattern, generating the final classification information by placing a first weight on the first classification information and a second weight on the second classification information, wherein the first weight and the second weight are different.

23. The method of claim 1, wherein:
the sensor data comprises (i) first sensor data for first one or more sensing cycles, and (ii) second sensor data for second one or more sensing cycles that occur subsequent to the first one or more sensing cycles;
the final classification information comprises
(i) first final classification information for the first one or more sensing cycles that are generated by (a) placing first weight on the first classification information associated with the first one or more sensing cycles and (b) second weight on the second classification information associated with the first one or more sensing cycles, and
(i) second final classification information for the second one or more sensing cycles that are generated by (a) placing third weight on the first classification information associated with the second one or more sensing cycles and (b) fourth weight on the second classification information associated with the second one or more sensing cycles; and
the first, second, third, and fourth weights are different.

24. The method of claim 23, wherein:
the first base caller implements a neural network model, and the second base caller does not include a neural network model;
the first weight is lower than the second weight, such that for the first one or more sensing cycles, the second classification information from the second base caller is emphasized more than the first classification information from the first base caller; and
the third weight is higher than the fourth weight, such that for the second one or more sensing cycles, the first classification information from the first base caller is emphasized more than the second classification information from the second base caller.

25. The method of claim 1, wherein:
the sensor data comprises (i) first sensor data from first one or more clusters of a tile of a flow cell, and (ii) second sensor data from second one or more clusters of the tile of the flow cell; the final classification information comprises:
(i) first final classification information for the first sensor data from the first one or more clusters, the first final classification information generated by (a) placing first weight on the first classification information from the first one or more clusters and (b) placing second weight on the second classification information from the first one or more clusters, and
(i) second final classification information for the second sensor data from the second one or more clusters, the second final classification information generated by (a) placing third weight on the first classification information from the second one or more clusters and (b) placing fourth weight on the second classification information from the second one or more clusters; and
the first, second, third, and fourth weights are different.

26. The method of claim 25, wherein:
the first one or more clusters are edge clusters that are disposed within a threshold distance from one or more edges of the tile of the flow cell; and
the second one or more clusters are non-edge clusters that are disposed beyond the threshold distance from the one or more edges of the tile of the flow cell.

27. The method of claim 1, further comprising:
detecting, from the sensor data, presence of one or more bubbles in at least one cluster of a tile of a flow cell,
wherein generating the final classification information comprises:
in response to the detection of the one or more bubbles, generating the final classification information by placing a first weight on the first classification information and a second weight on the second classification information, wherein the first weight and the second weight are different.

28. The method of claim 1, wherein the sensor data comprises at least one image, and wherein the method further comprises:
detecting that the at least one image is an out of focus image,
wherein generating the final classification information comprises:
in response to the detection of the out of focus image, generating the final classification information by placing a first weight on the first classification information and a second weight on the second classification information, wherein the first weight and the second weight are different.

29. A computer implemented method comprising:
generating sensor data for sensing cycles in the series of sensing cycles; and
executing at least a first base caller and a second base caller on at least corresponding portions of the sensor data, and selectively switching execution of the first and second base callers, based on context information associated with the sensor data, wherein the first base caller is different from the second base caller;
generating, by the first base caller and the second base caller, first classification information and second classification information, respectively,
generating base calls, based on one or both of the first classification information and the second classification information.

30. A non-transitory computer readable storage medium impressed with computer program instructions to progressively train a base caller, the instructions, when executed on a processor, implement a method comprising:
executing at least a first base caller and a second base caller on sensor data generated for sensing cycles in a series of sensing cycles;
generating, by the first base caller, first classification information associated with the sensor data, based on executing the first base caller on the sensor data;
generating, by the second base caller, second classification information associated with the sensor data, based on executing the second base caller on the sensor data; and
based on the first classification information and the second classification information, generating a final classification information, the final classification information including one or more base calls for the sensor data.
